(19) United States
(12) Patent Application Publication
(10) Pub. No.: US 2013/0274132 A1
(43) Pub. Date: Oct. 17, 2013

(54) Genetic Modifiers of Cystic Fibrosis

(71) Applicants: Lisa Strug, Toronto (CA); Lei Sun, Toronto (CA); Johanna M. Rommens, Toronto (CA); Garry Cutting, Towson, MD (US); Michael Knowles, Chapel Hill, NC (US); Mitchell L. Drumm, Brecksville, OH (US); Peter Durie, Toronto (CA)

(72) Inventors: Lisa Strug, Toronto (CA); Lei Sun, Toronto (CA); Johanna M. Rommens, Toronto (CA); Garry Cutting, Towson, MD (US); Michael Knowles, Chapel Hill, NC (US); Mitchell L. Drumm, Brecksville, OH (US); Peter Durie, Toronto (CA)

(73) Assignees: Hospital for Sick Children, Toronto, ON (CA); Case Western Reserve University, Cleveland, OH (US); Univeristy of North Carolina at Chapet Hill, Chapel Hill, NC (US); The Johns Hopkins University, Baltimore, MD (US)

(21) Appl. No.: 13/876,712

(22) PCT Filed: Sep. 30, 2011
(86) PCT No.: PCT/US11/54318

§ 371 Date: Jun. 4, 2013

Related U.S. Application Data
(60) Provisional application No. 61/388,782, filed on Oct. 1, 2010.
(60) Provisional application No. 61/405,079, filed on Oct. 20, 2010.
(60) Provisional application No. 61/394,963, filed on Oct. 20, 2010.
(60) Provisional application No. 61/405,005, filed on Oct. 20, 2010.

(51) Int. Cl. C12Q 1/68

(52) U.S. Cl. CPC C12Q 1/6883
USPC 506/9; 435/6.11

(57) ABSTRACT

Disclosed herein are compositions and methods for and treating Cystic Fibrosis lung disease severity and/or secondary manifestations, including meconium ileus and CF related liver disease.

### RELATED APPLICATIONS

This application claims the benefit of U.S. Provisional Application Ser. No. 61/388,782 filed on Oct. 1, 2010, U.S. Provisional Application Ser. No. 61/394,963, filed on Oct. 20, 2010, U.S. Provisional Application Ser. No. 61/405,005, filed on Oct. 20, 2010 and U.S. Provisional Application Ser. No. 61/405,079, filed on Oct. 20, 2010; the contents of each of which are hereby incorporated by reference in their entirety.

### STATEMENT OF GOVERNMENT SUPPORT

Aspects of the present invention were made with the support of funding under federal grant numbers K23DK083551, R01HL068927, R01HL68890 and R01DK66368 from the National Institutes of Health. The United States Government has certain rights to this invention.

### BACKGROUND

Cystic Fibrosis (CF) is a life-shortening recessive genetic disorder that is caused by mutations in the cystic fibrosis transmembrane conductance regulator (CFTR) gene. However, there is substantial variability in the clinical phenotypes of cystic fibrosis patients, even among individuals with the exact same loss-of-function CFTR mutation.

Individuals with cystic fibrosis often exhibit drastically different secondary disease manifestations and may have highly variable lung disease severity. For example, 16-25% of cystic fibrosis patients suffer from Meconium Ileus (MI) (a severe intestinal obstruction), while roughly 30% develop CF related diabetes (CFRD) in adulthood and 5-7% acquire cystic fibrosis related liver disease (CFLD). Furthermore, while almost all individuals with cystic fibrosis suffer from progressive bronchopulmonary disease, the severity and rate of decline in lung function highly variable among individuals carrying the same CFTR mutation.

Thus, there is great need for compositions and methods for the identification of individuals with cystic fibrosis who are at increased risk of severe lung disease, MI and/or CFLD.

### SUMMARY

In some embodiments, provided herein are methods of identifying a subject as having increased risk of severe lung disease. In some embodiments, the methods include the step of detecting in a biological sample from the subject an allele of a single nucleotide polymorphism. In certain embodiments the allele of the single nucleotide polymorphism is a) a C allele at single nucleotide polymorphism rs12793173, b) an A allele at single nucleotide polymorphism rs1403543, c) a C allele at single nucleotide polymorphism rs9268905, d) a G allele at single nucleotide polymorphism rs4760506, e) a T allele at single nucleotide polymorphism rs12883884, f) an A allele at single nucleotide polymorphism rs12188164, g) a C allele at single nucleotide polymorphism rs11645366 or h) any combination thereof. In some embodiments the methods include the step of detecting in a biological sample from the subject a variant of a gene. In some embodiments the gene is EHF, APIP, MC3R, CASS4, AURKA, CBLN4, C20orf106 and/or CSTF1.

In some embodiments, provided herein are methods of identifying subject as a carrier of an allele of a single nucleotide polymorphism associated with severe lung disease. In some embodiments, the methods include the step of detecting in a biological sample from the subject an allele of a single nucleotide polymorphism. In certain embodiments the allele of the single nucleotide polymorphism is a) a C allele at single nucleotide polymorphism rs12793173, b) an A allele at single nucleotide polymorphism rs1403543, c) a C allele at single nucleotide polymorphism rs9268905, d) a G allele at single nucleotide polymorphism rs4760506, e) a T allele at single nucleotide polymorphism rs12883884, f) an A allele at single nucleotide polymorphism rs12188164, g) a C allele at single nucleotide polymorphism rs11645366 or h) any combination thereof. In some embodiments the methods include the step of detecting in a biological sample from the subject a variant in a gene. In some embodiments the gene is EHF, APIP, MC3R, CASS4, AURKA, CBLN4, C20orf106 and/or CSTF1.

In some embodiments, provided herein are methods of identifying a subject as having increased risk of having cystic fibrosis liver disease (CFLD). In certain embodiments, the methods include the step of detecting in a biological sample from the subject an allele of a single nucleotide polymorphism. In certain embodiments the allele of the single nucleotide polymorphism is a) a C allele at single nucleotide polymorphism rs914232, b) a T allele at single nucleotide polymorphism rs2330183, c) a T allele at single nucleotide polymorphism rs2838956, d) a G allele at single nucleotide polymorphism rs1051266, e) a T allele at single nucleotide polymorphism rs4819130, f) a G allele at single nucleotide polymorphism rs3788190, g) a T allele at single nucleotide polymorphism rs2236483, h) a C allele at single nucleotide polymorphism rs2838950, i) a G allele at single nucleotide polymorphism rs12483377, j) a C allele at single nucleotide polymorphism rs3753019 or k) any combination thereof. In some embodiments the methods include the step of detecting in a biological sample from the subject a variant in a gene. In some embodiments the gene is SLC19A1 and/or COL18A1.

In some embodiments, provided herein are methods of identifying a subject as a carrier of an allele of a single nucleotide polymorphism associated with CFLD. In certain embodiments, the methods include the step of detecting in a biological sample from the subject an allele of a single nucleotide polymorphism. In certain embodiments the allele of the single nucleotide polymorphism is a) a C allele at single nucleotide polymorphism rs914232, b) a T allele at single nucleotide polymorphism rs2330183, c) a T allele at single nucleotide polymorphism rs2838956, d) a G allele at single nucleotide polymorphism rs1051266, e) a T allele at single nucleotide polymorphism rs4819130, f) a G allele at single nucleotide polymorphism rs3788190, g) a T allele at single nucleotide polymorphism rs2236483, h) a C allele at single nucleotide polymorphism rs2838950, i) a G allele at single nucleotide polymorphism rs12483377, j) a C allele at single nucleotide polymorphism rs3753019 or k) any combination thereof. In some embodiments the methods include the step of detecting in a biological sample from the subject a variant in a gene. In some embodiments the gene is SLC19A1 and/or COL18A1.

In some embodiments, provided herein are methods of identifying a subject as having increased risk of meconium ileus (MI). In some embodiments the methods include the step of detecting in a biological sample from the subject an allele of a single nucleotide polymorphism. In some embodiments the allele of the single nucleotide polymorphism is a) a C allele at single nucleotide polymorphism rs7512462, b) a G allele at single nucleotide polymorphism rs7415921, c) a G allele at single nucleotide polymorphism rs4077468, d) a T allele at single nucleotide polymorphism rs4077469, e) a G allele at single nucleotide polymorphism rs12047830, f) an A allele at single nucleotide polymorphism rs7419153, g) a T allele at single nucleotide polymorphism rs10179921, h) a T allele at single nucleotide polymorphism rs4684689, i) an A allele at single nucleotide polymorphism rs17563161, j) a T allele at single nucleotide polymorphism rs3788766, k) a C allele at single nucleotide polymorphism rs5905283, 1) a G allele at single nucleotide polymorphism rs12839137 or k) any combination thereof. In some embodiments the methods include the step of detecting in a biological sample from the subject a variant in a gene. In some embodiments the gene is SLC26A9, SLC6A14, SLC9A3, ABCG8 and/or ATP2B2.

In some embodiments, provided herein are methods of identifying a subject as a carrier of an allele of a single nucleotide polymorphism associated with MI. In some embodiments the methods include the step of detecting in a biological sample from the subject an allele of a single nucleotide polymorphism. In some embodiments the allele of the single nucleotide polymorphism is a) a C allele at single nucleotide polymorphism rs7512462, b) a G allele at single nucleotide polymorphism rs7415921, c) a G allele at single nucleotide polymorphism rs4077468, d) a T allele at single nucleotide polymorphism rs4077469, e) a G allele at single nucleotide polymorphism rs12047830, f) an A allele at single nucleotide polymorphism rs7419153, g) a T allele at single nucleotide polymorphism rs10179921, h) a T allele at single nucleotide polymorphism rs4684689, i) an A allele at single nucleotide polymorphism rs17563161, j) a T allele at single nucleotide polymorphism rs3788766, k) a C allele at single nucleotide polymorphism rs5905283, 1) a G allele at single nucleotide polymorphism rs12839137 or k) any combination thereof. In some embodiments the methods include the step of detecting in a biological sample from the subject a variant in a gene. In some embodiments the gene is SLC26A9, SLC6A14, SLC9A3, ABCG8 and/or ATP2B2.

In certain embodiments of the methods described herein, the subject lacks a wild-type CFTR gene, has or is suspected of having cystic fibrosis, is or is suspected of being a carrier of a mutated CFTR gene, and/or has at least one family member that has or is suspected of having cystic fibrosis.

In some embodiments, the methods described herein also includes the step of determining whether the biological sample lacks a wild-type CFTR gene. In certain embodiments, the methods described herein include the step of obtaining the biological sample from the subject.

In some embodiments of the methods described herein, the step of detecting includes performing a hybridization assay, an amplification assay and/or a nucleic acid sequencing assay.

In some embodiments of the methods described herein, the sample a tissue sample, a blood sample, a semen sample and/or a germ cell sample. In certain embodiments, the subject is a human adult, a human child, a human fetus, a human embryo or a human fertilized cell.

In some embodiments, described herein are methods of determining whether a test compound is a candidate therapeutic agent for reducing lung disease severity. In certain embodiments, the methods include a) contacting a cell with the test compound; and b) detecting the expression by the cell of a gene product of EHF, APIP, MC3R, CASS4, AURKA, CBLN4, C20orf106 and/or CSTF1.

In some embodiments, described herein are methods of determining whether a test compound is a candidate therapeutic agent for treating CFLD. In certain embodiments, the methods include a) contacting a cell with the test compound; and b) detecting the expression by the cell of a gene product of SLC19A1 and/or COL18A1.

In some embodiments, described herein are methods of determining whether a test compound is a candidate therapeutic agent for treating MI. In certain embodiments, the methods include a) contacting a cell with the test compound; and b) detecting the expression by the cell of a gene product of SLC26A9, SLC6A14, SLC9A3, ABCG8 and/or ATP2B2.

In some embodiments of the methods described herein, the gene product is an mRNA and/or a protein. In certain embodiments the gene product is linked to a detectable moiety. In some embodiments the expression of the gene product is detected by detecting the detectable moiety. In certain embodiments, the agent is a small molecule, a polypeptide, an antibody or an inhibitory RNA molecule.

In some embodiments, described herein are methods of reducing lung disease severity in a subject. In certain embodiments, the methods include administering to the subject a therapeutic agent that modulates the expression or activity of a gene product encoded by EHF, APIP, MC3R, CASS4, AURKA, CBLN4, C20orf106 and/or CSTF1.

In some embodiments, described herein are methods of treating and/or preventing CFLD in a subject. In certain embodiments, the methods include administering to the subject a therapeutic agent that modulates the expression or activity of a gene product encoded by SLC19A1 and/or COL18A1.

In some embodiments, described herein are methods of treating and/or preventing MI in a subject. In certain embodiments, the methods include administering to the subject a therapeutic agent that modulates the expression or activity of a gene product encoded by SLC26A9, SLC6A14, SLC9A3, ABCG8 and/or ATP2B2.

In certain embodiments of the methods described herein, the subject lacks a wild-type CFTR gene, has or is suspected of having cystic fibrosis, is or is suspected of being a carrier of a mutated CFTR gene, and/or has at least one family member that has or is suspected of having cystic fibrosis. In certain embodiments, the agent is a small molecule, a polypeptide, an antibody and/or an inhibitory RNA molecule. In some embodiments, the agent reduces the expression or activity of the gene product.

### BRIEF DESCRIPTION OF THE FIGURES

FIG. 1 is a table that provides of exemplary SNPs.

FIG. 2 is a table that provides the characteristics of patients enrolled by three studies in the North American CF Gene Modifier Consortium.

FIG. 3 shows histograms of the Consortium lung phenotype for the three cystic fibrosis studies show similar average phenotypes. The phenotype mean is above zero due to a lower bound placed by the survival correction, as well as cohort effects of improving lung function. (a) The two designs using unrelated individuals. All of the patients in the Genetic Modifier Study (GMS) are F508del/F508del at CFTR. These patients were oversampled at extremes of an initial entry phenotype, in order to improve power, and the original severe/mild designations are colored separately. In contrast, the Canadian Consortium for Genetic Studies (CGS) is population based, representing a range of pancreatic insufficient CFTR genotypes. (b) Patients enrolled in the family-based Twin and Sibling Study (TSS) show a similar distribution of the Consortium lung phenotype as the population-based CGS.

FIG. 4 shows Power analyses for genome-wide significance of P<5×10⁻⁸ for GMS and CGS F508del/F508del (n=1978). Each plotted point is the result of 1000 simulations using the phenotype-conditional sampling scheme described in Online Methods. (a) Results as a function of (i) the population effect size β₁, defined as the change in average Consortium lung phenotype for each additional copy of the minor allele, and (ii) minor allele frequency. The power depends essentially on the proportion of variation explained, as illustrated in (b). The power ranges from 25%-90% to detect variants explaining 1%-2% of population phenotype variation. Power for the combined scan of GMS and all CGS patients is even higher, due to the larger sample size for that scan.

FIG. 5 shows genome-wide Manhattan plots for the cystic fibrosis Consortium lung function phenotype, combining the association evidence from GMS and CGS samples across 570,725 SNPs. The black dashed line represents the Bonferroni threshold for genome-wide α=0.05, while the gray dashed line is the suggestive association threshold, expected once per genome scan. SNPs are plotted in Mb relative to their position on each chromosome (alternating gray and black) (a) Results from GMS (n=1137, all of whom are F508del/F508del) combined with all of the CGS patients (n=1357). Seven regions reach suggestive significance. (b) Results from the combined evidence of GMS (n=1137) and the CGS F508del/F508del (n=841). A region on chromosome 11p13 reaches genome-wide significance (p=3.34×10⁻⁸).

FIG. 6 is a table that provides significant and suggestive association results for GMS and CGS with replication values for TSS.

FIG. 7 shows an alternative analysis of association evidence for the Consortium lung phenotype shows consistent evidence for the 11p13 EHF/APIP region. Results from the conditional likelihood approach described in Online Methods applied to GMS+CGS F508del/F508del. The black and gray dashed lines correspond to genome-wide and suggestive significance, respectively.

FIG. 8 is a table that provides the covariate effects for significant and suggestive SNPs.

FIG. 9 shows (a) Joint analysis of association evidence from GMS and all patients from CGS and TSS shows that the 11p13 EHF/APIP region reaches genome-wide significance for this population set, and chromosome 6 region near HLA-DRA on 6p21 nearly achieves genome-wide significance. (b) Joint analysis of association evidence from GMS and the F508del/F508del from CGS and TSS shows striking evidence in the EHF/APIP region (P=8.28×10⁻¹° at rs568529). The black and gray dashed lines correspond to genome-wide and suggestive significance, respectively.

FIG. 10 shows a plot of the association evidence in GMS and CGS F508del/F508del in the chromosome 11p13 EHF/APIP region (NCBI build 36, LocusZoom viewer).

FIG. 11 shows Conditional analysis of the chromosome 11 association result for the Consortium lung phenotype, GMS+CGS F508del/F508del. After conditioning on the most significant SNP rs 12793173 from the genome scan the minimum p-value in the interval is 3.73×10⁻⁵ at SNP rs286873. The conditional result was significant for the interval (Bonferroni p=0.0029 for the 77 SNPs annotated to the EHF/APIP interval). After another step of conditioning on rs286873 alone, and performing another regional scan, the original SNP rs12793173 remained the most significant.

FIG. 12 shows an illustrative Manhattan plot for association of the Consortium lung phenotype with genoCNV copy number, at 2544 loci variable (variant frequency >1%) in a combined analysis of GMS and all CGS patients. No region attained Bonferroni genome-wide significance threshold for the 2544 CNV loci. The model shown here is trait=sex+PCs+cnⱼ+cnⱼ*sex+acⱼ*sex (Model ii in Online Methods). P-values were computed by comparing the full model to the reduced model with no copy number information, and combined across the two studies using Fisher's combined p-value method.

FIG. 13 shows genome-wide linkage scan for the Consortium lung phenotype in the family-based TSS cystic fibrosis study, adjusted for sex. A QTL with a genome-wide significant LOD=5.04 was found on 20q13.2. SNP used in the linkage panel are plotted in cM relative to their position on each chromosome (alternating gray and black).

FIG. 14 shows correlation plots of the lung function measures of sibling pairs who do not share either parental allele of rs4811626 on chromosome 20 (Identity by descent or IBD 0; 117 pairs), share one allele (IBD1; 248 pairs) or share both parental alleles (IBD2; 116 pairs). The IBD status could not be assigned for 5 sibling pairs. Correlation was calculated when sex and BMI were used as covariates. Correlation by IBD status was a follows: IBD0: r=0.1762, r²=0.0310; IBD1: r=0.4136 r²=0.171 and IBD2: r=0.6542, r²=0.428. The contribution of the linked region to variation was estimated by subtracting the correlation in siblings who are IBD 0 from the correlation in sibling who are IBD 2 (0.6542−0.1762=0.478). Similar estimates were obtained using variance components methods in Merlin (0.499) and SOLAR (0.496).

FIG. 15 shows regional analysis of the QTL on chromosome 20q13 (a) A detailed chromosome 20 linkage plot for the Consortium lung phenotype in the TSS study, with covariates sex (essentially the same result as for no covariates) and with covariates sex and BMI. (b) Association evidence from the GMS and CGS F508del/F508del patients, in the 1-LOD support interval provided by TSS.

FIG. 16 is a table that provides combined association and linkage-weighted FDR q-values and genome-wide ranks for SNPs with WFDR 1-values of genome wide significance (<0.05).

FIG. 17 shows a weighted false-discovery rate analysis (WFDR) is used to provide combined linkage evidence from TSS with association evidence from GMS+CGS F508del/F508del. A Manhattan plot portrays the ranks of the SNP WFDR q-values, with the rank for these data corresponding to q=0.05 shown as a dashed line. SNPs with q-values less than 0.05 are plotted above the dotted line. Note that the 11p13 and 20q13 regions are both significant, with WFDR q-values of 0.028 and 0.015, respectively.

FIG. 18 shows a proposed mechanism of stellate cell activation and fibrogenesis in CFLD. Hepatic stellate cell activation is amplified by the combined stimulus of hepatocytes, cholangiocytes and multiple stimuli that may reflect genetic factors (only a few of the known mediators are shown).

FIG. 19 shows a Manhattan plot of SNP p-values in CFLD. The top dotted line is threshold for genome-wide significance and the bottom dotted line is “suggestive” (i.e., only expect one SNP above this line by chance).

FIG. 20 shows SNPs on chromosome 21 (Chr 21) that associate with CFLD, and plotted relative to known genes and recombination rates.

FIG. 21 is a table that provides details of the CF consortium participants of the MI study.

FIG. 22 shows a QQ-plot of the MI genome-wide association (GWAS) analysis performed via a GEE model.

FIG. 23 is a table that provides the sex-specific results for rs3788766.

FIG. 24 shows a regional plot of the association evidence for MI around the solute carrier protein gene, SLC6A14, on chromosome X.

FIG. 25 is a set of tables from the MI study that provide (a) GWAS results for all CF patients, (b) GWAS results for ΔF508 Homozygous CF patients, and (c) OR estimates for all SNPs in (a) with a q-value <0.05.

FIG. 26 is a table that provides a list of genes that encode proteins that localize to the apical plasma membrane.

FIG. 27 shows a QQ-plot of SNPs in apical plasma membrane genes (A) and SNPs in other genes (B).

FIG. 28 shows a QQ-plot of SNPs from apical plasma membrane genes (A) and nuclear envelope genes (B) based on observed data and permuted phenotype data under the null hypothesis of no association.

FIG. 29 shows a trace plot of SNP association in the CFTR region (A) and a regional plot of the association evidence in and around CFTR (B).

FIG. 30 is a table that provides the results of the GWAS-HD analysis for MI.

FIG. 31 shows a regional plot of the MI association evidence in and around SLC26A9.

FIG. 32 shows the polymorphism in the CEBPB binding site of the SLC6A14 promoter region.

FIG. 33 shows a QQ-plot from the GWAS for the combined statistic to detect pleiotropic effects for MI and lung disease.

FIG. 34 is a table that provides the top 16 SNPs (p-value ≦10⁻⁵) according to the genome-wide MI association qq-plot shown in FIG. 33.

FIG. 35 shows that MI GWAS leads to genome-wide significant SNPs. (a) Genome-wide Manhattan plot for MI. The black solid line represents the genome-wide significance threshold⁷ (P value<5×10⁻⁸), and the black dashed line is the suggestive association threshold, expected once per genome scan. (b) Regional plot for SLC26A9. LocusZoom viewer was used to generate and display the association evidence around SLC26A9 based on NCBI Build 36/hg18. (c) Regional plot for SLC6A14.

FIG. 36 is a table that provides information regarding MI-associated SNPs in SLC26A9 and SLC6A14.

FIG. 37 is a table that provides the sex-specific results for rs3788766 and rs5905283 in SLC6A14.

FIG. 38 shows genome-wide MI association results with and without adjusting for the effect of CFTR. The x-axis shows the association P values (on the −log10 scale) of the original GWAS with the site covariate but without adjusting for the effect of CFTR as in FIG. 1a; the y-axis shows the association P values with both the site covariate and the CFTR covariate for which Phe508del/Phe508del genotype is coded as 1 and Phe508del/Other or Other/Other genotypes are coded as 0. SNPs within 155 kb of CFTR have been removed from this figure, and the SNPs at the bottom-left that have some noticeable discrepancy between the two sets of analyses are the SNPs that are in LD with CFTR.

FIG. 39 is a table that provides MI-association results for SNPs in SLC6A14 and SLC26A9 with and without adjustment for CFTR.

FIG. 40 shows that the apical membrane hypothesis identifies genes associated with MI. (a) QQ-plot of the apical SNPs in the discovery sample. (b) Statistical significance of the apical membrane hypothesis in the discovery sample. Statistical significance (permutation P=0.0002) was established via a sum statistic, summing the association evidence (Wald χ₁² statistic) over all the 3,814 SNPs with observed sum statistic. (c) QQ-plot of the apical SNPs in the replication sample. (d) Statistical Significance of the apical membrane hypothesis in the replication sample (permutation P=22/1,000=0.022).

FIG. 41 is a table that provides gene-based and Lasso association results for SLC6A14 and 157 Apical Genes.

FIG. 42 shows a GWAS-HD flow chart.

FIG. 43 is a table that provides ranked SNPs with MI association with q values <0.05 from GWAS or GWAS-HD.

FIG. 44 shows assessment of the nuclear envelope null hypothesis. (a) QQ-plot of the nuclear envelope gene SNPs in the discovery sample. (b) Statistical significance of the nuclear envelope hypothesis in the discovery sample.

### DETAILED DESCRIPTION

### I. General

Despite the fact that cystic fibrosis (CF) is considered a “monogenic” recessive disease caused by the mutation of the CFTR gene, there is substantial variability in CF clinical phenotype, even among individuals carrying the exact same CFTR mutations. Provided herein are genetic markers (e.g., SNP alleles and gene variants) associated with increased risk of severe lung disease, cystic fibrosis related liver disease (CFLD) and/or meconium ileus (MI) in individuals with cystic fibrosis. As described herein, such SNPs and gene variants are useful, for example, in methods of identifying a subject (e.g., a subject who has or is suspected of having CF) as having an increased risk of severe lung disease, CFLD and/or MI. Such genetic markers are also useful for the identification of individuals who carry genetic modifiers of cystic fibrosis clinical phenotype, the identification of novel therapeutic agents and for the treatment of lung disease, CFLD and/or MI.

Also described herein are therapeutic targets which can be modulated in order to treat and/or prevent cystic fibrosis, severe lung disease, CFLD and/or MI. Such therapeutic targets are also useful for the identification of novel therapeutic agents for the treatment of cystic fibrosis, severe lung disease, CFLD and/or MI.

### II. Definitions

In order that the present invention may be more readily understood, certain terms are first defined. Additional definitions are set forth throughout the detailed description.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “agent” is used herein to denote a chemical compound, a small molecule, a mixture of chemical compounds, a biological macromolecule (such as a nucleic acid, an antibody, a protein or portion thereof), or an extract made from biological materials such as bacteria, plants, fungi, or animal (particularly mammalian) cells or tissues. Agents may be identified as having a particular activity (e.g. modulating a therapeutic target) by screening assays described herein below. The activity of such agents may render them suitable as a “therapeutic agent” which is a biologically, physiologically, or pharmacologically active substance (or substances) that acts locally or systemically in a subject.

As used herein, an “allele” refers to one of two or more alternative forms of a nucleotide sequence at a given position (locus) on a chromosome. An individual can be heterozygous or homozygous for any allele of described herein.

The term “altered level of expression” or “modulated expression” of a gene product (e.g., a therapeutic target described herein) refers to an expression level of a gene product in a cell or sample that has been contacted with an agent that is greater or less than the expression level of the same gene product a control cell or sample (e.g., a cell or sample of the same type that has not been contacted with the agent or that has been contacted with a placebo agent).

The term “altered activity” or “modulated activity” of a gene product (e.g., a therapeutic target described herein) refers to an activity level of a gene product in a cell or sample that has been contacted with an agent that is greater or less than the activity level of the same gene product a control cell or sample (e.g., a cell or sample of the same type that has not been contacted with the agent or that has been contacted with a placebo agent). Altered activity may be the result of, for example, altered mRNA level, altered protein level, altered structure, altered ligand binding, and interference with protein-protein interactions.

As used herein, the term “antibody” includes full-length antibodies and any antigen binding fragment (i.e., “antigen-binding portion”) or single chain thereof. The term “antibody” includes, but is not limited to, a glycoprotein comprising at least two heavy (H) chains and two light (L) chains inter-connected by disulfide bonds, or an antigen binding portion thereof. Antibodies may be polyclonal or monoclonal; xenogeneic, allogeneic, or syngeneic; or modified forms thereof (e.g., humanized, chimeric).

As used herein, the term “cystic fibrosis” or “CF” describes a recessive genetic disorder that manifests in individuals who have two bona fide mutations in trans in the cystic fibrosis transmembrane conductance regulator (CFTR) gene. The mRNA and protein sequences of wild-type CFTR are provided at GenBank® accession numbers NM_{—}000492.3 and NP_{—}000483.3, respectively. Cystic fibrosis causing mutations in the CFTR gene are well known in the art. The most common CFTR mutation is the ΔF508 mutation.

As used herein, the phrases “gene product” and “product of a gene” refers to a substance encoded by a gene and able to be produced, either directly or indirectly, through the transcription of the gene. The phrases “gene product” and “product of a gene” include RNA gene products (e.g. mRNA), DNA gene products (e.g. cDNA) and polypeptide gene products (e.g. proteins).

The terms “increased risk” or “increased likelihood” as well as “decreased risk” or “decreased likelihood” as used herein define the level of risk or the likelihood that a subject has or will develop severe lung disease, CFLD, or MI, as compared to a control subject that does not carry one or more of the alleles of a single nucleotide polymorphism or the mutated genes described herein.

As used herein, a “marker”, “genetic marker,” “polymorphic marker” or “polymorphism” is a genomic DNA sequence associated with and individual at increased risk for severe lung disease, CFLD or MI. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population.

As used herein, the term “modulation” refers to up regulation (i.e., activation or stimulation), down regulation (i.e., inhibition or suppression) of the expression of a gene product, of a biological activity, or the two in combination or apart.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

“Sample,” “tissue sample,” “subject sample,” or “biological sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject. The tissue sample may contain compounds that are not naturally intermixed with the tissue in nature such as preservatives, anticoagulants, buffers, fixatives, nutrients, antibiotics or the like.

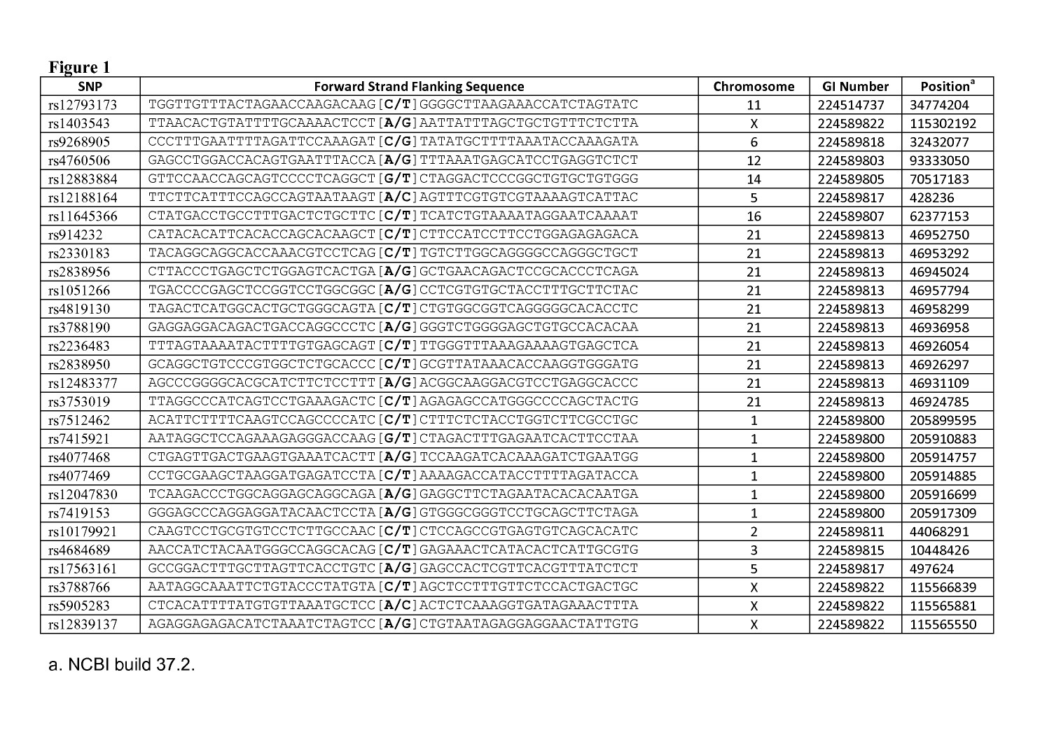

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI). A SNP allele can be describe based on the sequence of its forward strand or the sequence of its reverse strand. For example, a SNP that has either A or G alleles on its forward strand will have either T or C alleles, respectively, on its reverse strand. The SNP alleles are described herein according to their forward strand sequence. Exemplary SNPs are provided in FIG. 1, along with their forward strand flanking sequences and their chromosomal position.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

As used herein, the terms “subject” and “subjects” refer to an animal, e.g., a mammal including a non-primate (e.g., a cow, pig, horse, donkey, goat, camel, cat, dog, guinea pig, rat, mouse, sheep) and a primate (e.g., a monkey, such as a cynomolgous monkey, gorilla, chimpanzee and a human). In some embodiments, the subject may be a human adult, a human child, a human fetus, a human embryo and/or a human fertilized cell.

As used herein, the term “target” or “therapeutic target” are used interchangeably and refer to a gene product whose activity and/or expression can be modulated in order to treat and/or prevent a disease or disorder.

The phrases “therapeutically-effective amount” and “effective amount” as used herein means that amount of a therapeutic agent which is effective for producing some desired therapeutic effect in at least a sub-population of cells in an animal at a reasonable benefit/risk ratio applicable to any medical treatment.

“Treating” a disease in a subject or “treating” a subject having a disease refers to subjecting or exposing the subject to a pharmaceutical treatment, e.g., the administration of a drug, such that at least one symptom of the disease is decreased or prevented from worsening.

As used herein, the terms “variant of a gene,” “gene variant,” “mutation of a gene” and “gene mutation” are used interchangeably and refer to a particular allele of a gene described herein that is associated with increased risk for a disease or disorder. The variant may be functional or non-functional. The variant or mutation may be the gene allele that is less prevalent among the general population, but, in some instances, the variant or mutation may be the allele that is more prevalent among the general population.

### III. SNPs and Gene Variants Associated with Lung Disease Severity

Lung disease is the major source of morbidity and mortality for patients afflicted with cystic fibrosis (CF), a recessive disorder caused by mutations in the cystic fibrosis transmembrane conductance regulator (CFTR) gene. The identification of CFTR and its disease-causing mutations provided substantial insight into the molecular pathophysiology of CF, but allelic variation in CFTR does not explain the wide variation in severity of lung disease. Therefore, identification of the genetic modifiers would increase our understanding of CF disease progression, suggest new targets for intervention and could identify potential mechanisms for variation in lung function in CF, as well as, for common chronic respiratory diseases such as COPD.

Provided herein are predictive alleles of single nucleotide polymorphisms associated with an increased risk of severe lung disease. As described herein, such alleles are therefore genetic markers of an increased risk of severe lung disease and are useful, for example, in the methods described herein for the identification of individuals as having increased risk of severe lung disease. These alleles include, but are not limited to, a C allele at single nucleotide polymorphism rs12793173, an A allele at single nucleotide polymorphism rs1403543, a C allele at single nucleotide polymorphism rs9268905, a G allele at single nucleotide polymorphism rs4760506, a T allele at single nucleotide polymorphism rs12883884, an A allele at single nucleotide polymorphism rs12188164 and/or a C allele at single nucleotide polymorphism rs11645366. In certain embodiments, combinations comprising one or more of the predictive alleles are used in the methods described herein. These single nucleotide polymorphisms are identified by a reference number that can be found in the publicly available GenBank® database, well known to those of skill in the art.

Also provided herein are genes associated with severe lung disease in individuals with cystic fibrosis. Like the alleles of single nucleotide polymorphisms described above, variants of such genes are genetic markers of an increased risk of severe lung disease and are therefore useful, for example, in the methods described herein for the identification of individuals as having increased risk of severe lung disease. Furthermore, such the products of such genes are also therapeutic targets for the treatment of sever lung disease. As such, they are useful, for example, in the methods described herein for the treatment of severe lung disease and in the methods described herein for the identification of therapeutic agents for the treatment of severe lung disease. In some embodiments, agents that modulate the activity and/or expression of such therapeutic targets are identified as candidate therapeutic agents useful in the reduction in the severity of lung disease in individuals with cystic fibrosis. Furthermore, in certain embodiments, modulation of the activity and/or expression of such therapeutic targets are used to lung disease severity in individuals with cystic fibrosis.

Severe lung disease associated genes provided herein include EHF, APIP, MC3R, CASS4, AURKA, CBLN4, C20orf106 and CSTF1. The following GenBank® Database Accession numbers provide the wild-type sequences for the mRNA and protein encoded by each of these genes:
EHF: NP_{—}001193544.1 (protein); NM_{—}001206615.1 (mRNA).
APIP: NP_{—}057041.2 (protein); NM_{—}015957.2 (mRNA).
AURKA: NP_{—}940835.1 (protein); NM_{—}198433.1 (mRNA).
CBLN4: NP_{—}542184.1 (protein); NM_{—}080617.4 (mRNA).
C20orf106: NP_{—}001012989.2 (protein); NM_{—}001012971.3 (mRNA).
CSTF1: NP_{—}001028693.1 (protein); NM_{—}001033521.1 (mRNA).

In certain embodiments, the gene variants described herein include any mutation which modulates the activity and/or expression of a product of the severe lung disease associated gene. For example, such mutations can be insertion, deletion and/or substitution mutations. In certain embodiments, the mutation is a loss of function mutation. In some embodiments the mutation is a frame shift mutation and/or a truncation. In certain embodiments the variant is not a mutation to a coding portion of the severe lung disease associated gene, but rather to a transcription control element operably linked to the gene. For example, in some embodiments, the mutation is to a promoter or enhancer of the severe lung disease associated gene.

A subject can be homozygous or heterozygous for one or more of the genetic markers described herein, in any combination. For example, a subject can be homozygous for one marker and heterozygous for another marker and such homozygous and/or heterozygous markers can be present in any combination in a subject.

### IV. SNPs and Gene Variants Associated with Cystic Fibrosis Liver Disease (CFLD)

There is currently no way to identify children at risk for CFLD and the molecular pathogenesis is not currently understood. Because CFLD develops early in life (median age ˜10 years), “preventive” intervention in this irreversible process would need to be undertaken early. Furthermore, since only 5% of CF patients develop CFLD, there is currently no feasible study design to test potential therapies.

Provided herein are predictive alleles of single nucleotide polymorphisms associated with an increased risk of CFLD. As described herein, such alleles are therefore genetic markers of an increased risk of CFLD and are useful, for example, in the methods described herein for the identification of individuals as having increased risk of CFLD. These alleles include, but are not limited to, a C allele at single nucleotide polymorphism rs914232, a T allele at single nucleotide polymorphism rs2330183, an A allele at single nucleotide polymorphism rs2838956, a G allele at single nucleotide polymorphism rs1051266, a T allele at single nucleotide polymorphism rs4819130, a G allele at single nucleotide polymorphism rs3788190, a T allele at single nucleotide polymorphism rs2236483, a C allele at single nucleotide polymorphism rs2838950, a G allele at single nucleotide polymorphism rs12483377, and/or a C allele at single nucleotide polymorphism rs3753019. In certain embodiments, combinations of the predictive alleles are used in the methods described herein. These single nucleotide polymorphisms are identified by a reference number that can be found in the publicly available GenBank® database, well known to those of skill in the art.

Also provided herein are genes associated with CFLD. Like the alleles of single nucleotide polymorphisms described above, variants to such genes are genetic markers of an increased risk of CFLD and are therefore useful, for example, in the methods described herein for the identification of individuals as having increased risk of CFLD. Furthermore, the products of such genes are also therapeutic targets for the treatment of CFLD. As such, they are useful, for example, in the methods described herein for the treatment of CFLD and in the methods described herein for the identification of therapeutic agents for the treatment of CFLD. In some embodiments, agents that modulate the activity and/or expression of such therapeutic targets are identified as candidate therapeutic agents for the prevention and/or treatment of CFLD. Furthermore, in certain embodiments, modulation of the activity and/or expression of the therapeutic targets described herein are used to prevent and/or treat CFLD.

CFLD associated genes provided herein include SLC19A1 and COL18A1. The following GenBank® Database Accession numbers provide the wild-type sequences for the mRNA and protein encoded by each of these genes:
SLC19A1: NP_{—}919231.1 (protein); NM_{—}194255.1 (mRNA).
COL18A1: Isoform 1: NP_{—}085059.2 (protein); NM_{—}030582.3 (mRNA).
   Isoform 2: NP_{—}569711.2 (protein); NM_{—}130444.2 (mRNA).
   Isoform 3: NP_{—}569712.2 (protein); NM_{—}130445.2 (mRNA).

In certain embodiments, the gene variants described herein include any mutation which modulates the activity and/or expression of a product of the CFLD associated gene. For example, such mutations can be insertion, deletion and/or substitution mutations. In certain embodiments, the mutation is a loss of function mutation. In some embodiments the mutation is a frame shift mutation and/or a truncation. In certain embodiments the variant is not a mutation to a coding portion of the CFLD associated gene, but rather to an transcription control element operably linked to the gene. For example, in some embodiments the mutation is to a promoter or enhancer of the CFLD associated gene.

A subject can be homozygous or heterozygous for one or more of the genetic markers described herein, in any combination. For example, a subject can be homozygous for one marker and heterozygous for another marker and such homozygous and/or heterozygous markers can be present in any combination in a subject.

### V. SNPs and Gene Variants Associated with Meconium Ileus

Meconium ileus (MI), a type of intestinal blockage, is seen in 16-25% of CF patients at birth with an equal sex ratio, but is otherwise rare such that presence of MI at birth is highly indicative of CF. Furthermore, MI almost exclusively occurs in patients with two severe CF-causing CFTR mutations (−87% of all patients). Although food digestion does not occur in utero, various facets of the gastrointestinal tract do begin to function including production of digestive enzymes, with clearance of material being essential immediately after birth. CFTR is expressed in various segments of the small and large intestine. With the loss of CFTR, the meconium (or first stool in the newborn) is altered as the intestinal mucus secretions that begin in utero are abnormally sticky and adherent leading to a blockage of the latter portion of the small intestine. The proximal ileum can be enlarged and the subsequent distal ileum and the colon may appear collapsed. The obstructions are dense material comprised of a mixture of bile salts, bile acids and debris that is typically shed from the intestinal mucosa during the fetal period. Intestinal obstruction due to MI will be evident as early as 24-48 hours after birth with distention, vomiting and failure to pass meconium. Intervention to remove the blockage is required immediately, via an enema procedure or by surgical intervention.

Provided herein are predictive alleles of single nucleotide polymorphisms associated with an increased risk of MI. As described herein, such alleles are therefore genetic markers of an increased risk of MI and are useful, for example, in the methods described herein for the identification of individuals as having increased risk of MI. These alleles include, but are not limited to, a C allele at single nucleotide polymorphism rs7512462, a G allele at single nucleotide polymorphism rs7415921, a G allele at single nucleotide polymorphism rs4077468, a T allele at single nucleotide polymorphism rs4077469, a G allele at single nucleotide polymorphism rs12047830, an A allele at single nucleotide polymorphism rs7419153, a T allele at single nucleotide polymorphism rs10179921, a T allele at single nucleotide polymorphism rs4684689, an A allele at single nucleotide polymorphism rs17563161, a T allele at single nucleotide polymorphism rs3788766, a C allele at single nucleotide polymorphism rs5905283 and/or a G allele at single nucleotide polymorphism rs12839137. In certain embodiments, combinations of the predictive alleles are used in the methods described herein. These single nucleotide polymorphisms are identified by a reference number that can be found in the publicly available GenBank® database, well known to those of skill in the art.

Also provided herein are genes associated with MI. Like the alleles of single nucleotide polymorphisms described above, variants of such genes are genetic markers of an increased risk of MI and are therefore useful, for example, in the methods described herein for the identification of individuals as having increased risk of MI. Furthermore, the products of such genes are therapeutic targets for the treatment of MI. As such, they are useful, for example, in the methods described herein for the treatment of MI and in the methods described herein for the identification of therapeutic agents for the treatment of MI. In some embodiments, agents that modulate the activity and/or expression of such therapeutic targets are identified as candidate therapeutic agents for the prevention and/or treatment of MI. Furthermore, in certain embodiments, modulation of the activity and/or expression of such therapeutic targets are used to prevent and/or treat MI.

MI associated genes provided herein include SLC26A9, SLC6A14, SLC9A3, ABCG8 and ATP2B2. The following GenBank® Database Accession numbers provide the wild-type sequences for the mRNA and protein encoded by each of these genes:
SLC26A9: NP_{—}443166.1 (protein); 1.NM_{—}052934.3 (mRNA).
SLC6A14: NP_{—}009162.1 (protein); NM_{—}007231.3 (mRNA).
SLC9A3: NP_{—}004165.2 (protein); NM_{—}004174.2 (mRNA).
ABCG8: NP_{—}071882.1 (protein); NM_{—}022437.2 (mRNA).
ATP2B2: NP_{—}001001331.1 (protein); NM_{—}001001331.2 (mRNA).

In certain embodiment, the gene variants described herein include any mutation which modulates the activity and/or expression of a product of the MI associated gene. For example, such mutations can be insertion, deletion and/or substitution mutations. In certain embodiments, the mutation is a loss of function mutation. In some embodiments the mutation is a frame shift mutation and/or a truncation. In certain embodiments the variant is not a mutation to a coding portion of the MI associated gene, but rather to a transcription control element operably linked to the gene. For example, in some embodiments the mutation is to a promoter or enhancer of the MI associated gene.

A subject can be homozygous or heterozygous for one or more of the genetic markers described herein, in any combination. For example, a subject can be homozygous for one marker and heterozygous for another marker and such homozygous and/or heterozygous markers can be present in any combination in a subject

### VI. Identification of Subjects with Increased Risk of Severe Lung Disease, CFLD and MI

Described herein are methods of identifying a subject who has an increased risk of severe lung disease, CFLD and/or MI. In certain embodiments, the method includes the step of detecting in a biological sample from a subject a genetic marker described herein. In some embodiments, the genetic marker is an allele of a single nucleotide polymorphism that is associated with severe lung disease, CFLD and/or MI. In some embodiments, the genetic marker is a variant of a gene that is associated with severe lung disease, CFLD and/or MI. In some embodiments, the method comprises a combination of any one or more genetic markers described herein are detected. In general, if the genetic marker is detected in the biological sample, the subject from whom the biological sample was obtained has an increased risk of severe lung disease, CFLD and/or MI. In certain embodiments, the subject has or is suspected of having cystic fibrosis. For example, in certain embodiments, the subject lacks a wild-type CFTR gene. In some embodiments, the subject has at least one family member that has or is suspected of having cystic fibrosis. In some embodiments, the method also includes the step of obtaining the biological sample from the subject.

In certain embodiments, the methods described herein also include the step of detecting mutated and/or wild-type CFTR in the sample. As described herein, individuals with cystic fibrosis carry mutations in both copies of their CFTR gene. Thus, in certain embodiments, the methods described herein determine both whether the subject has cystic fibrosis and whether the subject is at increased risk of severe lung disease, CFLD and/or MI.

Mutation of a single CFTR gene in a subject results in the subject being a carrier of the cystic fibrosis mutation. When two CFTR mutation carriers have a child, there is a one in four chance that the child will have cystic fibrosis. It is therefore desirable to know both whether an individual is a carrier of a cystic fibrosis causing mutation, but also whether an individual is a carrier of a genetic marker described herein.

Thus, described herein are methods of identifying a subject as a carrier of a genetic marker associated with severe lung disease, CFLD and/or MI. In certain embodiments, the method includes the step of detecting in a biological sample from a subject a genetic marker described herein. In some embodiments, the genetic marker is an allele of a single nucleotide polymorphism that is associated with severe lung disease, CFLD and/or MI. In some embodiments, the genetic marker is a variant of a gene that is associated with severe lung disease, CFLD and/or MI. In some embodiments, a combination of the genetic markers described herein are detected. In general, if the genetic marker is detected in the biological sample, the subject from whom the biological sample was obtained is a carrier of a genetic marker associated with severe lung disease, CFLD and/or MI. In some embodiments, the subject has at least one family member that has or is suspected of having cystic fibrosis. In certain embodiments, the subject is a carrier of a CFTR mutation. In some embodiments, the method also includes the step of obtaining the biological sample from the subject.

In some embodiments, of the methods described herein, the subject will be a human child or a human adult. In some embodiments, the subject will be an infant. However, in certain embodiments the subject is not limited to being a fully developed human. Thus, in some embodiments, the subject will be a human fetus, a human embryo and/or a human fertilized cell.

Any type of biological sample that contains genetic material can be used in the methods described herein. Thus, for example, in some embodiments the sample is a cell, a body fluid, a swabbing, a tissue sample, a blood sample and/or a germ cell sample.

Any method known in the art can be used to detect the genetic markers described herein and/or the CFTR gene. Thus, in certain embodiments, the detecting step includes performing a hybridization assay (e.g., SNP or gene microarrays, dynamic allele-specific hydridization (DASH), TaqMAN, HPA, scorpion probes and molecular beacons), performing a nucleic acid amplification assay (e.g., PCR, LCR, TMA, SDA, NASBA, BDA, 3SR, RCR, etc.) and/or performing a nucleic acid sequencing assay.

In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3SR), QP replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well known methods, such as hybridization with an allele-specific probe, secondary amplification with allele-specific primers, by restriction endonuclease digestion, and/or by electrophoresis. Thus, also provided herein are oligonucleotides for use as primers and/or probes for detecting and/or identifying genetic markers according to the methods described herein.

Additional methods for detecting the genetic markers described herein include sequencing, high performance liquid chromatography (HPLC), restriction enzyme analysis (e.g., restriction fragment length polymorphism or RFLP), hybridization, matrix assisted laser desorption/ionization-time of flight mass spectrometry (MALDI-TOF-MS), etc., all of which are well known protocols for analyzing a nucleotide sequence and detecting genetic markers. The methods described herein can be carried out by using any assay or procedure that can interrogate a nucleic acid sequence.

In some embodiments, detecting can be carried out by an amplification reaction and single base extension, and in further embodiments, the product of the amplification reaction and single base extension can be spotted on a silicone chip according to methods well known in the art.

Prior to analyzing the sample, it may be necessary to process the sample to yield a form acceptable for analysis. For example, the nucleic acid (e.g. genomic DNA) may be extracted from the sample using techniques well-established in the art including chemical extraction techniques utilizing phenol-chloroform, guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN (Chatsworth, Calif.), or the Extract-N-Amp blood kit available from Sigma (St. Louis, Mo.).

In certain embodiments, also provided herein is a kit comprising reagents to detect one or more of the markers described herein in a biological sample from a subject. Such a kit can comprise primers, probes, primer/probe sets, reagents, buffers, etc., as would be known in the art, for the detection of the genetic markers described herein in a biological sample from a subject. For example, a primer or probe can comprise a contiguous nucleotide sequence that is complementary (e.g., fully (100%) complementary or partially (50%, 60%, 70%, 80%, 90%, 95%, etc.) complementary) to a region comprising a marker described herein. In particular embodiments, a kit described herein can comprise primers and probes that allow for the specific detection of at least 1, 2, 3, 4, 5, 6, 7, 8, 9, 10, 11, 12, 13, 14, 15, 16, 17, 18, 19 or 20 of the markers described herein. Such a kit can further comprise blocking probes, labeling reagents, blocking agents, restriction enzymes, antibodies, sampling devices, positive and negative controls, etc., as would be well known to those of skill in the art.

Also provided herein are methods of identifying an effective and/or appropriate (i.e., for a given subject's particular condition or status) treatment regimen for a subject with increased risk of severe lung disease, CFLD and/or MI, that includes detecting one or more of the genetic markers described herein in the subject, wherein the one or more genetic markers are further statistically correlated with an effective and/or appropriate treatment regimen for cystic fibrosis, severe lung disease, CFLD and/or MI according to protocols as described herein and as are known in the art.

Also provided is a method of identifying an effective and/or appropriate treatment regimen for a subject with increased risk of severe lung disease, CFLD and/or MI, that includes: a) correlating the presence of one or more genetic markers described herein in a test subject or population of test subjects with severe lung disease, CFLD and/or MI for whom an effective and/or appropriate treatment regimen has been identified; and b) detecting the one or more genetic markers of step (a) in the subject, thereby identifying an effective and/or appropriate treatment regimen for the subject.

Further provided is a method of correlating one or more genetic markers described herein with an effective and/or appropriate treatment regimen for severe lung disease, CFLD, and/or MI that includes: a) detecting in a subject or a population of subjects with severe lung disease, CFLD and/or MI and for whom an effective and/or appropriate treatment regimen has been identified, the presence of one or more genetic markers described herein; and b) correlating the presence of the one or more genetic markers of step (a) with an effective treatment regimen for severe lung disease, CFLD or MI.

Examples of treatment/management regimens for severe lung disease, CFLD and MI are well known in the art. Subjects who respond well to particular treatment protocols can be analyzed for specific genetic markers and a correlation can be established according to the methods provided herein. Alternatively, subjects who respond poorly to a particular treatment regimen can also be analyzed for particular genetic markers correlated with the poor response. Then, a subject who is a candidate for treatment for severe lung disease, CFLD and/or MI can be assessed for the presence of the appropriate genetic markers and the most effective and/or appropriate treatment regimen can be provided.

In some embodiments, the methods of correlating genetic markers with treatment regimens described herein can be carried out using a computer database. Thus, in some embodiments, provided herein is a computer-assisted method of identifying a proposed therapy and/or treatment for CFLD as an effective and/or appropriate therapy and/or treatment for a subject that has CFLD, comprising the steps of: (a) storing a database of biological data for a plurality of subjects, the biological data that is being stored including for each of said plurality of subjects: (i) therapy and/or treatment type, (ii) at least one genetic marker described herein, and (iii) at least one disease progression measure and/or symptom for severe lung disease, CFLD and/or MI from which treatment and/or therapy efficacy can be determined; and then (b) querying the database to determine the dependence on said genetic marker(s) of the effectiveness of a treatment and/or therapy type in treating and/or managing severe lung disease, CFLD, and/or MI, thereby identifying a proposed treatment and/or therapy as an effective and/or appropriate treatment and/or therapy for a subject with increased risk of severe lung disease, CFLD and/or MI.

Nonlimiting examples of disease progression measures and/or symptoms that can be monitored to determine efficacy can be determined including all of the complications and symptoms of CF, CFLD and MI as described herein and would be well known in the art.

In one embodiment, treatment information for a subject is entered into the database (through any suitable means such as a window or text interface), genetic marker information for that subject is entered into the database, and disease progression responsiveness to treatment information is entered into the database. These steps are then repeated until the desired number of subjects has been entered into the database. The database can then be queried to determine whether a particular treatment is effective for subjects carrying a particular marker or combination of markers, not effective for subjects carrying a particular marker or combination of markers, etc. Such querying can be carried out prospectively or retrospectively on the database by any suitable means, but is generally done by statistical analysis in accordance with known techniques, as described herein.

### VII. Therapeutic Agents

Certain methods described herein relate to the administration of an agent that modulates the activity and/or expression of a therapeutic target described herein. Agents which may be used to modulate the expression or activity of a therapeutic target described herein include antibodies (e.g., conjugated antibodies), proteins, peptides, small molecules and inhibitory RNA molecules, e.g., siRNA molecules, shRNA, ribozymes, and antisense oligonucleotides. Such agents can be those described herein, those known in the art, or those identified through routine screening assays (e.g. the screening assays described herein).

In some embodiments, an assay is used to identify agents useful in the therapeutic methods described herein. For example, provided herein are methods of determining whether a test compound is a candidate therapeutic agent for reducing lung disease severity, treating CFLD and/or treating MI. In general, such methods include (a) contacting a cell with the test compound and (b) detecting the expression by the cell of therapeutic target described herein (e.g. a therapeutic target associated with severe lung disease, CFLD and/or MI). A test compound that modulates the expression of a therapeutic target (for example, compared to cells treated with a placebo or untreated cells) is a candidate therapeutic agent.

Any cell can be used in the above described screening method. For example, in some embodiments the cell is a human cell. Cells used in the screen can be primary cells or a cell line. Examples of other cell lines useful in the screening assays described herein include, but are not limited to, 293-T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells.

The expression of the therapeutic targets described herein can be detected using any method known in the art. For example, the expression of the therapeutic target can be detected by detecting therapeutic target mRNA using, e.g., a detectably labeled nucleic acid probe, RT-PCR, and/or microarray technology. The expression of the therapeutic target can also be detected by detecting the therapeutic target protein using, e.g., detectably labeled antibodies that have binding specificity for the therapeutic target.

In some embodiments, a cell is used in the screening assay that has been genetically engineered to facilitate the performance of the assay. For example, in some embodiments, the cell is engineered such that the therapeutic target is expressed as a heterologous protein linked to a detectable moiety (e.g. a fluorescent moiety such as GFP or a luminescent moiety such as luciferase). In other embodiments, the cell contains a nucleic acid sequence encoding a detectable moiety operably linked to the promoter of the therapeutic target. In such embodiments, rather than detecting expression of the therapeutic target, the expression of the detectable moiety is detected directly. Such cells can be generated using standard recombinant techniques well known in the art.

Agents useful in the methods of the present invention may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

### VIII. Pharmaceutical Compositions

The agents described herein (e.g. agents that modulate the expression or activity of a therapeutic target described herein) can be incorporated into pharmaceutical compositions suitable for administration to a subject. The compositions may contain a single such agent or any combination of modulatory agents described herein and a pharmaceutically acceptable carrier. The pharmaceutical composition may further comprise additional agents useful for treating severe lung disease, CFLD, and/or MI.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, intravenous, intradermal, subcutaneous, oral, transdermal (topical), transmucosal, and rectal administration.

Toxicity and therapeutic efficacy of the agents described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Appropriate dosage agents depends upon a number of factors within the scope of knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

### 1× Therapeutic Methods

In some embodiments, described herein are methods for treating cystic fibrosis, reducing lung disease severity, treating and/or preventing severe lung disease, treating and/or preventing CDLD and/or treating and/or preventing MI by administering to a subject (e.g. a subject in need thereof) an agent described herein (e.g. an agent that modulates the expression or activity of a therapeutic target described herein).

A subject in need thereof may include, for example, a subject who has or is suspected of having cystic fibrosis, a subject who lacks a wild-type CFTR gene, a subject who has a family history of CFTR and, e.g., a subject who has at least one family member that has or is suspected of having cystic fibrosis. A subject in need thereof may also be an individual having increased risk of severe lung disease, CFLD and/or MI, as determined, for example, using the methods described herein. A subject in need thereof may be a subject who carries one or more of the genetic markers described herein.

In some embodiments, the subject will be administered a pharmaceutical composition described herein. In certain embodiments the pharmaceutical composition will incorporate a therapeutic agent in an amount sufficient to deliver to a patient a therapeutically effective amount of the therapeutic agent as part of a prophylactic or therapeutic treatment. The desired concentration of the active agent will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of the subject agent may be determined by reference to the plasma concentrations of the agent. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents of the invention employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

This invention is further illustrated by the following examples which should not be construed as limiting. The contents of all references, patents and published patent applications cited throughout this application, as well as the Figures, are incorporated herein by reference.

### EXAMPLES

### Example 1

### Modifier Loci of Lung Disease Severity in Cystic Fibrosis

### Genome-Wide Association Analysis of Lung Disease Severity in CF

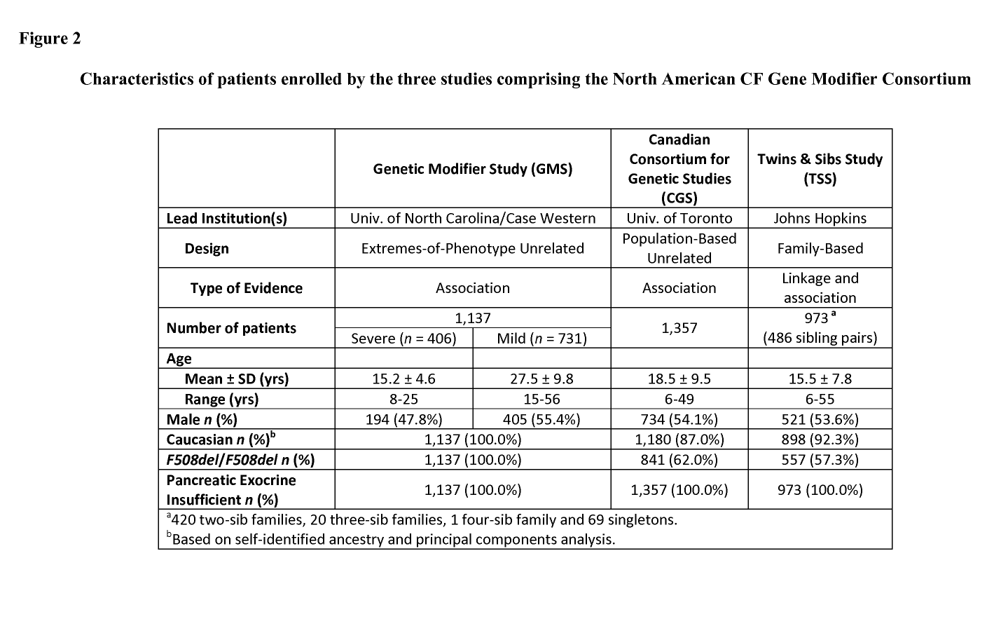

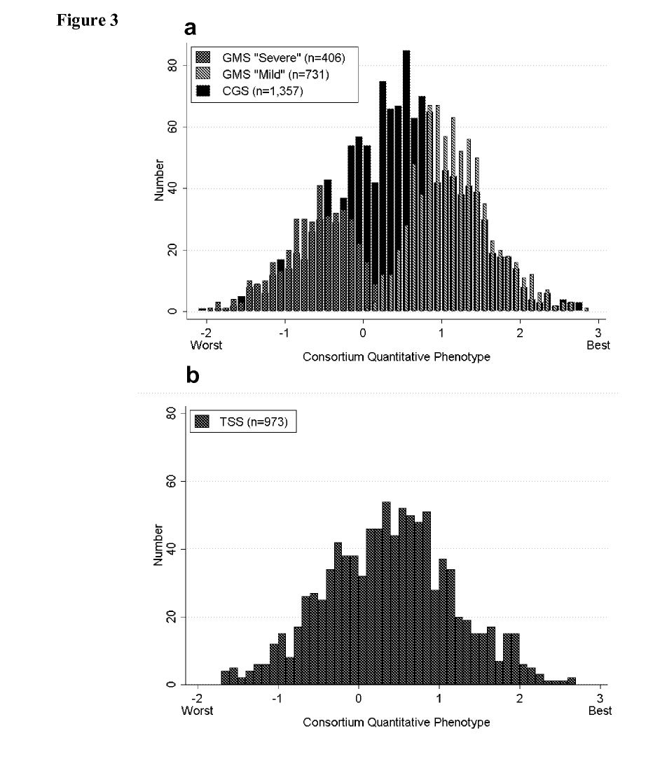

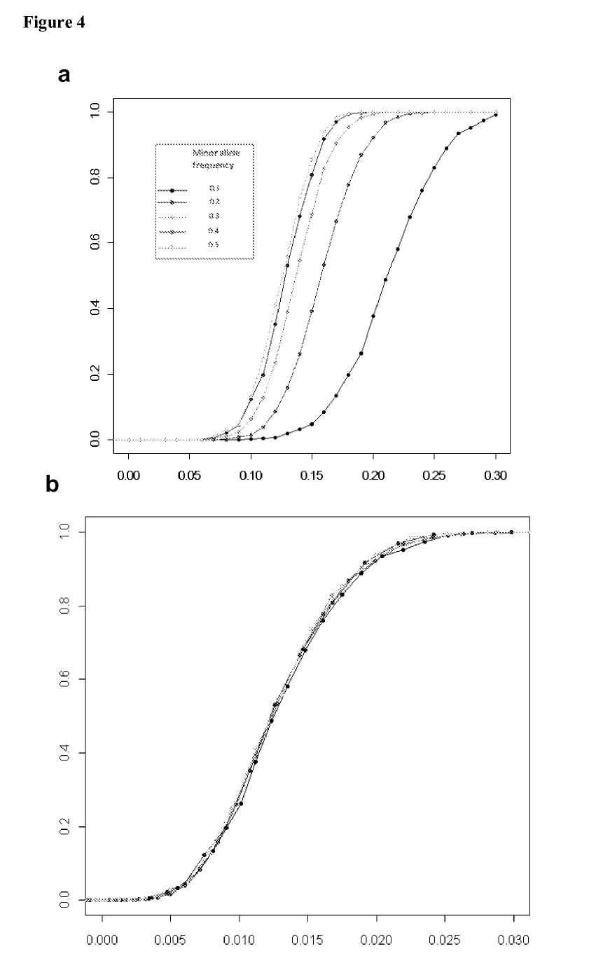

A total of 3,467 CF patients are represented in three study designs (FIG. 2). All patients in the GMS and 60% of the patients in the CGS and TSS populations are F508del homozygotes (denoted as F508del/F508del), while the remainder has a variety of other severe exocrine pancreatic CFTR genotypes. The three samples of CF patients showed consistent distributions of the lung disease phenotype, with the mid-range under-represented in GMS due to the extremes-of-phenotype design (FIG. 3). Genotyping was performed using the Illumina 610-Quad Array® for all patients contemporaneously in a single facility under stringent quality control (see Methods Section below). Association scans for the unrelated patient designs (GMS and CGS) used an additive model adjusted for sex and with principal component correction for population substructure. Genome-wide results were combined using a directional meta-analysis approach for GMS and CGS under two different conditions for CFTR mutation status: (1) GMS and CGS, n=2,494 and (2) GMS and CGS F508del/F508del, n=1,978. The meta-analysis approach provided over 90% power to detect a SNP responsible for >2% of phenotypic variation in an additive model (FIG. 4).

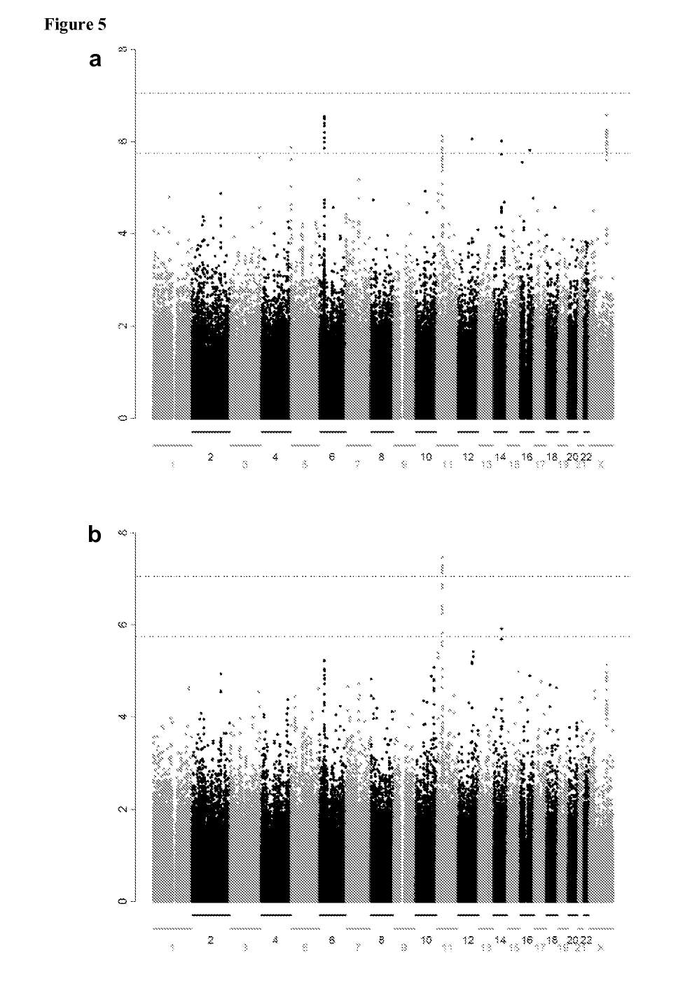

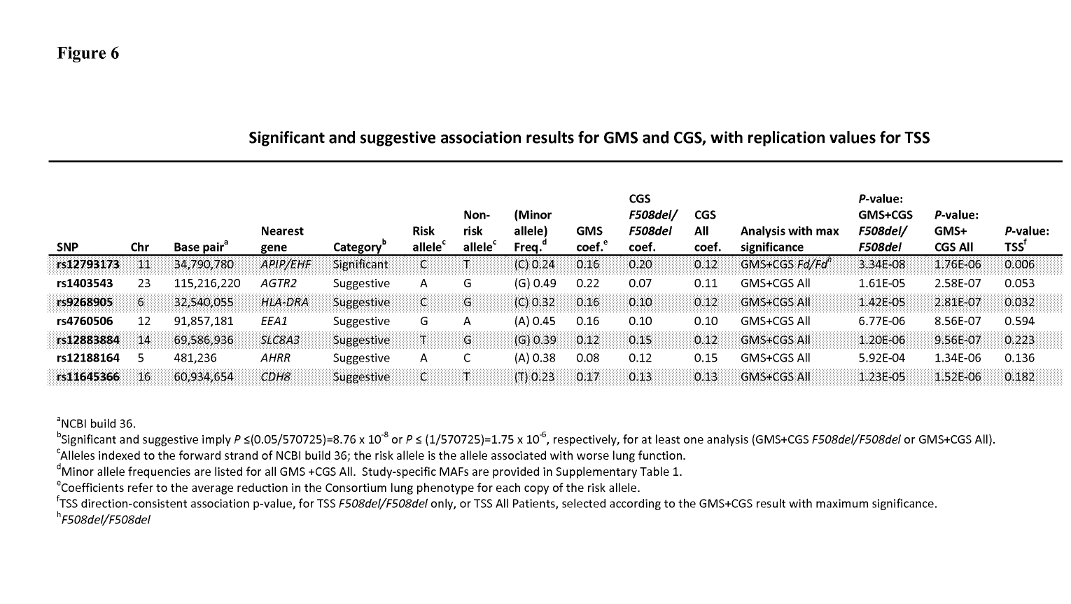

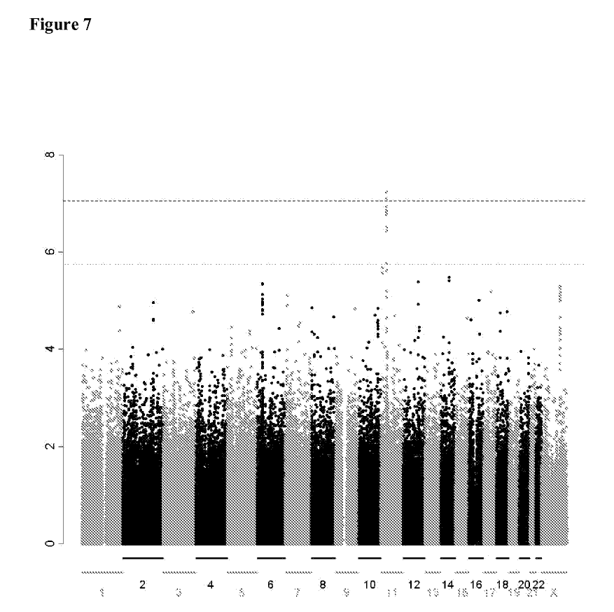

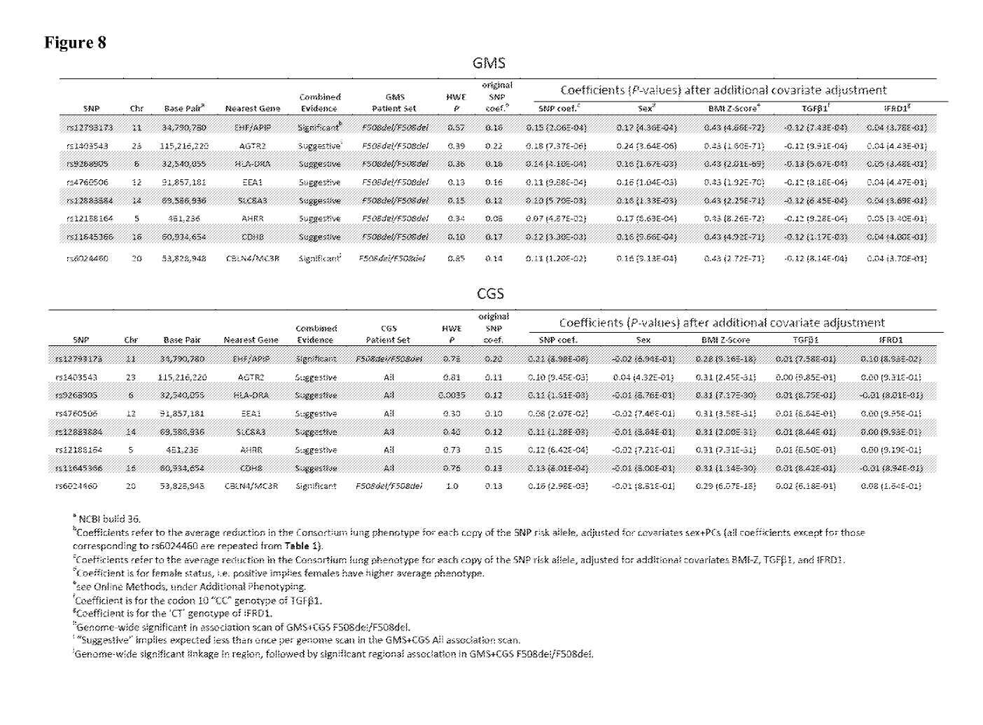

The combined analysis of GMS and CGS samples identified seven regions that achieved suggestive association (defined as P≦1/570,725=1.75×10⁻⁶) (FIGS. 5 and 6). When the analysis was restricted to patients with the same CFTR genotype (GMS and CGS F508del/F508del), the EHF-APIP region on chromosome 11p13 had genome-wide significance at rs12793173 (P=3.34×10⁻⁸, percentage of explained phenotype variation 1.0% in GMS, and 2.2% in CGS F508del/F508del). To further evaluate this approach, an alternative conditional likelihood approach was developed which explicitly acknowledged the selection of extremes of the lung phenotype in the GMS sample and extensive permutation analysis were performed to produce empirical p-values under the null hypothesis. Both approaches verified the genome-wide significance of the chr11p13 finding (See Methods Section and FIG. 7). The statistical significance of rs12793171 was even stronger with the inclusion of several covariates shown to be related to CF lung disease (P=9.42×10-9 by meta-analysis of GMS and CGS F508del/F508del; FIG. 8).

The seven SNPs demonstrating association in the GMS and CGS samples were evaluated for association in the TSS sample using Merlin [5429], which accounts for family structure in a variance component framework. To be consistent with the allelic effect from the combined GMS and CGS samples, each replication test was one-sided, and the TSS patient sample for each SNP (all or F508del/F508delpatients) was chosen to be consistent with the GMS and CGS patient sample set providing maximum significance. Covariates for sex and 4 principal components were included in the TSS analysis. The chromosome 11 SNP that had genome-wide significance in GMS and CGS (rs12793173, F508del/F508del) demonstrated significant association in the TSS sample (P=0.006; Bonferroni corrected P=0.041 for the seven replication tests; FIG. 6). Two regions of suggestive association had less significant findings in the TSS sample: rs9268905 near HLA-DRA (P=0.032) and rs1403543 near AGTR2 (P=0.053).

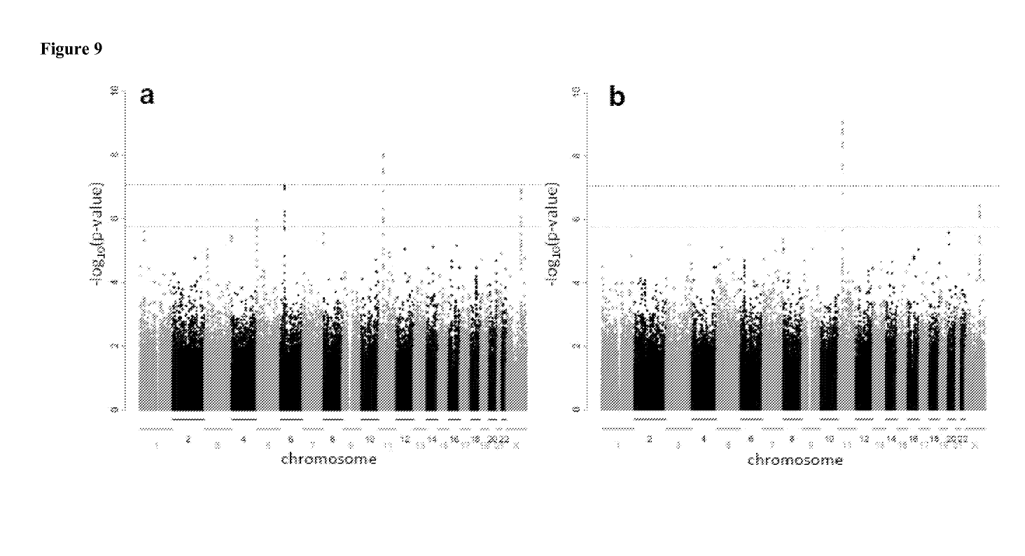

A joint analysis was performed. Using all available patients, rs568529, a SNP in high LD (r2>0.9) with rs12793173 in the EHF-APIP region, attained genome-wide significance (P=9.75×10-9). As in the earlier analysis, restricting to patients with the same CFTR genotype (F508del/F508del) increased the significance of the association between CF lung disease severity and the EHF-APIP region (rs568529, P=8.28×10-10). In the HLA class II region, a SNP (rs2395185) that is ˜1 kb from the suggestive SNP rs9268905 identified from GMS and CGS, approached genome-wide significance using all patients (P=9.02×10-8) (FIG. 9). SNPs in AGTR2 remained suggestive for all patients (rs5952206, P=1.25×10-7) and for F508del/F508del patients (rs7060450, P=3.67×10-7).

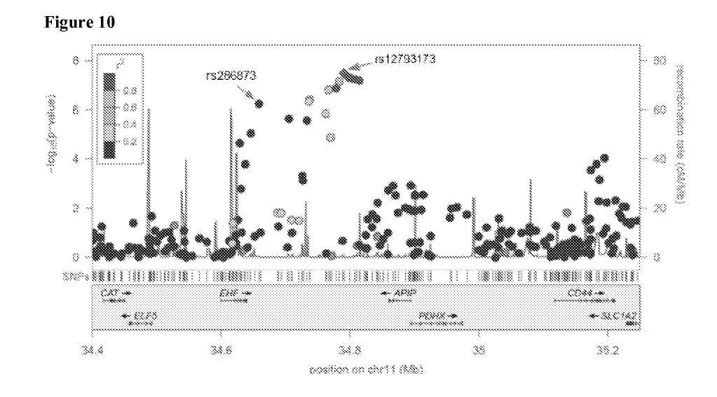

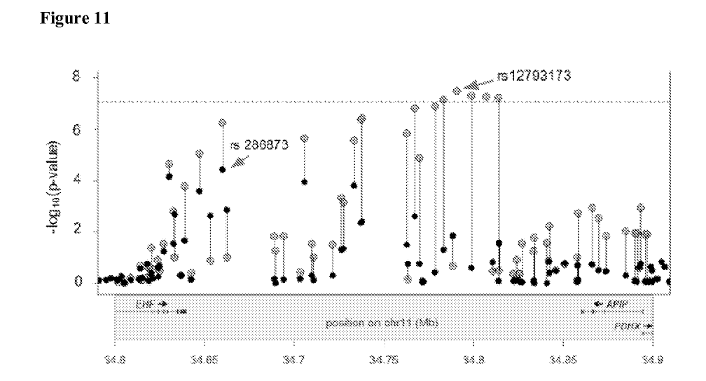

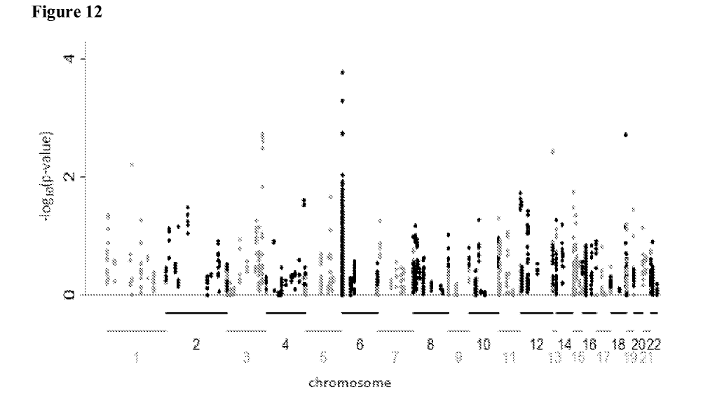

FIG. 10 shows 800 kb surrounding the EHF-APIP region in detail for the genotyped SNPs. The minimum P-value was achieved in an intergenic region 3′ to both EHF and APIP. A second peak at rs286873 (P=5.62×10-7) close to EHF exhibited low linkage disequilibrium (r2<0.2) with the primary association finding (FIG. 10). Multiple-SNP testing was performed to explore whether the second peak harbored other variants associated with the lung disease phenotype. After conditioning on the primary finding, rs286873 had regional statistical significance (rs12793173; regional corrected P=0.0029; FIG. 11) suggesting that several genetic variants may be responsible for association in the EHF-APIP region. Finally, MACH imputation was used to determine if any other variants not typed by the Illumina SNP array associated with lung function. Analysis of the imputed SNPs in the region identified the same EHF/APIP interval, with minimum P=1.45×10-8, at rs535719, at a position 19 kb closer to APIP than the genotyped SNP rs12793173. As an adjunct to testing of SNP genotype association, testing of association of the Consortium lung phenotype was performed with copy number variation, for models using estimates of both total copy number and allele-specific copy number (Online Methods). None of these models met genome-wide significance for the regions with variant frequency >1% (illustrative Manhattan plot in FIG. 12).

### Linkage of Lung Disease Severity in CF to Chromosome 20q13.2

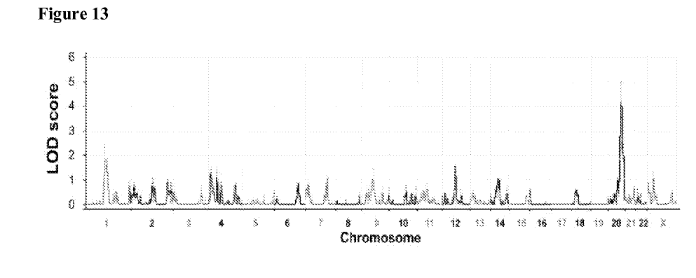

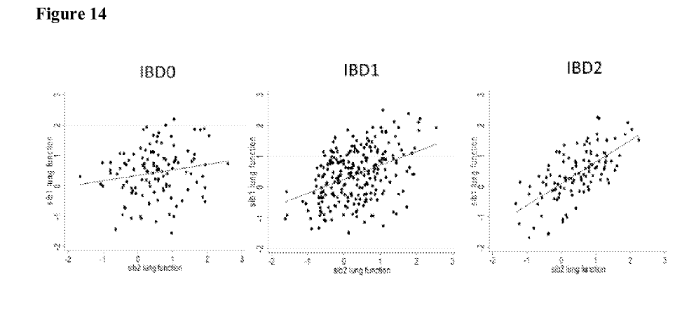

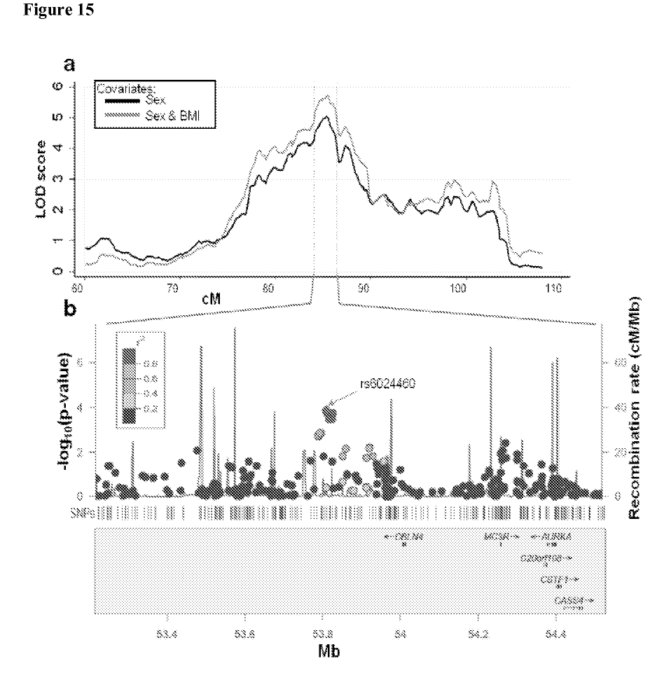

Linkage analysis of 486 sibling pairs using 19,566 SNPs selected from the Illumina array revealed a genome-wide significant multipoint LOD score of 5.033 at rs4811626, located at 53.81 Mb (−85cM) on chromosome 20q13.2 (nominal P=7.9×10-7; genome-wide P=2.3×10-3). Another noteworthy linkage signal was on chromosome 1p22.21, with multipoint LOD score of 2.48 for rs941031 at 91.07 Mb (119 cM). As body mass index is an important covariate of CF lung function (FIG. 8) and measures of human obesity have been linked to this region of chromosome 20, linkage analysis was performed using an average body mass index Z score(BMI-Z) as a covariate. Inclusion of BMI-Z increased the LOD score for the linkage peak on 20q13.2 (FIG. 13; the highest multipoint was for rs4811645 (LOD=5.72; genome-wide P=5.05e-04), which is 0.07 cM (0.13 Mb) from and immediately adjacent to rs4811626 on the linkage panel) and revealed suggestive linkage (rs1990398; LOD 2.21) on chromosome Xp21 at 27.41 Mb (43 cM). In contrast, the linkage peak on chromosome 1p22.21 decreased to LOD 1.67 when BMI was used as a covariate. Using three different methods, it was determined that the QTL on chromosome 20 may account for almost 50% of the variation in lung function in the CF sibling pairs (FIG. 14), however this estimate is likely to be biased upward due to winner's curse. A region of 1.314 Mb on 20q13.2, demarcated by 1 LOD unit below the maximum (when BMI-Z was used as a covariate), was selected for further analysis to determine if any SNPs within the chromosome 20 linkage peak demonstrated association with lung function in the combined GMS and CGS samples. In the center of the region (encompassing 307 SNPs, rs2225092 to rs6999), a cluster of SNPs 3′ of CBLN4 generated the lowest P-values in the combined GMS and CGS F508del/F508del samples (FIG. 15, lower panel). These SNPs (rs6092179, rs6024437, rs8125625, rs6024454 and rs6024460) displayed high LD with each other (r2>0.8) and were located in a 30 kb region (53.79 Mb to 53.82 Mb). The SNP with the lowest P-value (rs6024460; P=1.34×10-4) was significant for the region (Bonferroni corrected P=0.041). Importantly, the cluster of SNPs exhibiting allelic association with lung function in the unrelated patient samples are found in the same LD block as the SNPs with the highest LOD scores in the family-based sample (see above).

### A Combined False Discovery Rate Approach Corroborates Genome-Wide Significance of Loci on Chromosomes 11 and 20

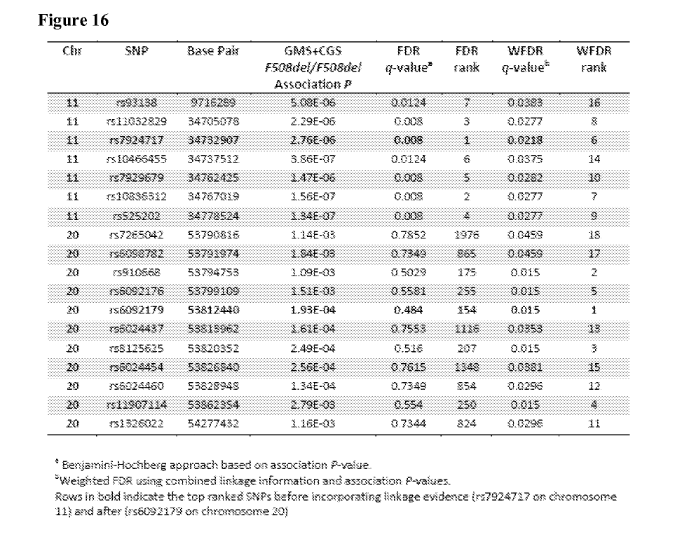

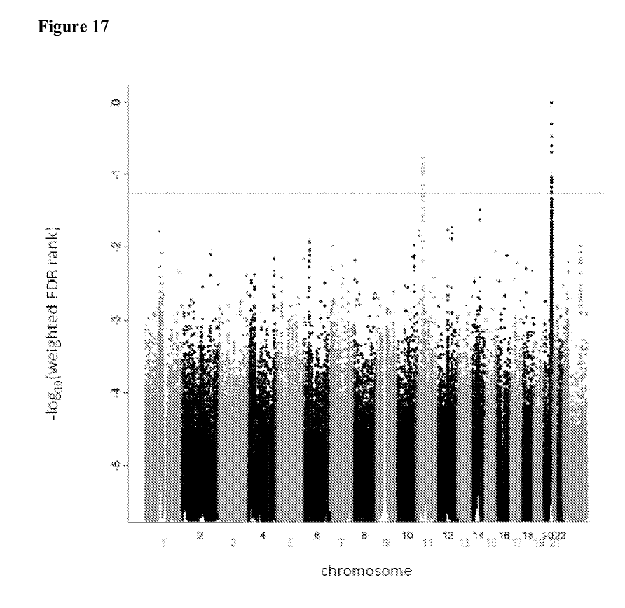

To evaluate association and linkage results in a single genome-wide framework, a combined analysis was performed that integrated the linkage data from TSS into the association data from GMS and CGS F508del/F508del. In essence, linkage information was used to reprioritize genome-wide association results using extensions of the false discovery rate (FDR) control methodology via the stratified FDR (SFDR) and weighted FDR (WFDR). The linkage-weighted q-values (genome-wide adjusted P-values that control FDR) reflective of the combined evidence for a gene modifier at a given SNP, within the GWAS design were obtained, and GWAS results after accounting for the linkage evidence were re-ordered. Presented herein are data from the WFDR; all results were confirmed using the SFDR. SNPs with q-values less than 0.05 were declared to be genome-wide significant (FIG. 16). SNPs in the EHF-APIP region on chromosome 11 are highly significant with or without the linkage information, because of the initial strong evidence for association. In contrast, the q-values for SNPs under the linkage peak on chromosome 20 change considerably after accounting for the linkage signal. The results presented in FIG. 16 illustrate that the linked SNPs on chromosome 20 are now top ranked genome-wide, while they were ranked 154th or lower by the GWAS evidence alone, prior to incorporating the linkage information. A rank-based Manhattan plot of the combined results demonstrates that chromosome 11 and chromosome 20 both provide genome-wide significant results (FIG. 17).

### Methods

Recruitment.

The TSS sample consisted of 486 affected sibling pairs (904 CF patients: 420 families with 2 siblings deriving 420 pairs, 20 families with 3 siblings deriving 60 pairs & 1 family with 4 siblings deriving 6 sibling pairs) recruited by the TSS. An additional 69 singletons from the TSS study were included for association analysis. All TSS patients and families were recruited based on having a surviving affected sibling as previously described. Written informed consent was obtained from all patients over 18 years of age. Parental or guardian consent was obtained for patients less than 18 years old along with assent from patients between 6 and 17 years old. Studies were approved by the Institutional Review Boards of Johns Hopkins University, UNC, CW and the Research Institute at the Hospital for Sick Children, Toronto, Canada.

Lung Disease Severity Phenotyping.

In CF, FEV1 is recognized as producing the most clinically useful measurements of lung function and a known predictor of survival. However, comparison of disease severity by FEV1 across a broad range of ages is confounded by the decline in FEV1 over time in CF patients, and by mortality attrition. In brief, age-specific CF percentile values of FEV1 were calculated for each patient using 3 years of data in patients 6 year or older, using the Kulich-derived U.S. (national) CF percentiles (relative to other CF patients of the same age, sex, and height, and then adjusted for CF age-specific mortality. The resulting quantitative phenotypes were distributed as expected based on ascertainment (FIG. 3). This quantitative phenotype was also highly correlated with the Schluchter survival phenotype (r2=0.91), when compared for the GMS patients.

Additional Phenotyping.

All patients carried severe CFTR mutations on both alleles that are known to confer pancreatic insufficiency. Sex was based on self-report and consistent genotype. Average BMI Z-score, used to stratify patients by nutritional status, was derived from the body mass index (kg/m2) calculated from height and weight measurements over the same time period (3 years duration) used to calculate the lung function phenotype. Standard deviation scores (Z-scores) were then calculated using CDC reference equations. After removal of 0.2% of data points due to inconsistent height or weight values, the resulting values were averaged to produce the BMI-Z covariate.

Genotyping and Quality Control.

DNA derived from either whole blood or transformed lymphocyte cell lines was hybridized to the Illumina 610-Quad genotyping platform at Genome Quebec facilities (McGill University and Genome Quebec Innovation Centre, Montreal) using the 96-well plate format. The plates containing the DNA samples were loaded at the respective lead institutions with a balance of sex and lung severity. Two CEPH DNA controls and one randomly chosen replicate sample were included per plate for quality control. Illumina BeadStudio software was used to call genotype. Sample identity was confirmed by comparing SNP calls to a Sequenom fingerprinting panel. Any discrepancies were resolved or rerun. Further quality control for SNPs and samples was conducted as summarized below.

The quality of the SNP calls was judged to be very high, with the discordance rate between duplicate samples calculated at 0.004% in GMS, and similar for the other studies. SNPs monomorphic across the studies were removed from analysis. SNPs were also removed if they showed a missing data rate >10%. Hardy-Weinberg testing was not used as an initial SNP filter, to allow discovery of true associations that might exhibit departures from equilibrium. The trios (mother, father, child) within TSS offered the opportunity to estimate SNP call error rates, and missing data rates. Using these trios, error rates from homozygote to heterozygote, homozygote to other homozygote, and heterozygote to homozygote were calculated, and SNPs with >1% Mendelian error rates were removed from analyses in all studies. Finally, 570,725 SNPs from autosomes and the X chromosome were selected for analyses, as well as 158 SNPs on chromosome Y and 138 mitochondrial SNPs.

Patient samples were excluded if the call rate in an initial screen was below 98%. Identity by state and identity by descent inferences were used to identify unexpected relatives in the datasets and samples with duplicate enrollment or plated in more than one study. Sex was confirmed by counting heterozygous X-chromosome genotypes and the number of called Y-chromosome genotypes. Unresolved sex mismatches and aneuploid patients were excluded. Samples were also examined to ensure no sample had an excess or deficit of heterozygous SNP calls more than 5 standard deviations from the mean heterozygosity of 31.6%. Additionally, quality was also assessed using a subset of the UNC cohort (917 patients) that had been previously genotyped for 1536 SNPs in candidate genes using Illumina GoldenGate technology. Of these SNPs, 542 were also found to give high quality SNP calls on the Illumina 610-Quad. Discordance between the genotype calls across the two platforms was 0.07%. For family-based samples, Mendelian consistency was checked for all trios. Samples and families with more than 5% Mendelian errors were excluded. In all, 28 (GMS6; CGS 17, TSS 5) patient samples were excluded from analysis due to genotyping failure or apparent artifacts, 2 GMS samples were excluded due to outlying ancestry (as evidenced by PC analysis) and 8 GMS samples were excluded for excessive (>second degree relation) of identify-by-descent proportions with other samples in the study. All of the reported significant and suggestive loci were subjected to additional scrutiny using Illumina GenomeStudio V1.0.2 genotyping module V1.0.10. with the GenTrainl clustering algorithm and manually-assisted genotyping to ensure high-quality calling and minimal potential impact of copy-number variation.

Genome-Wide Association Testing.

Regression analyses for the common lung phenotype were performed separately for the three study samples GMS, all CGS patients, and the CGS F508del/F508del patients. PLINK v. 1.07 (Am J Hum Genet. 2007 September; 81(3):559-75) was used for each analysis, using an additive genetic model while adjusting for sex and genotype-derived principal components (PCs).

For the GMS and CGS samples, PCs were obtained from SMARTPCA using a thinned set of ˜70,000 markers, derived separately for each study sample/subset, as described in Li et al. (Clin Genet. 2010). Eigenvalue analysis resulted in the choice of 4 PCs for the relatively homogeneous GMS sample, and 7 PCs for each of the CGS study samples. To acknowledge potentially differing standard errors across the sample designs due to the extremes-of-phenotype GMS design, a standard weighted meta-analysis z-statistic (Houwelingen et al., Statist. Med. 2002; 21:589-624) was constructed as a combined association statistic for primary GWAS analyses (GMS and CGS, GMS and CGS F508del/F508del). Using each of the genetic effect z-statistics for the GMS and CGS samples, the combined statistic was z=w_{GMS}z_{GMS}+w_{CGS}^{z}_{CGS}, with weights inversely proportional to the standard errors, and a common reference allele to ensure directional consistency of risk effects. Suggestive association was declared for P-values lower than the approximate threshold 1/(number of SNPs)=1/570,725=1.75×10-6. Significant association was declared using the conservative Bonferroni threshold P<0.05/570,725=8.76×10-8. Slight variation in the number of informative markers for various analyses was of no consequence in declaring significance. Similarly, genotypes among males of X-chromosome SNPs were encoded according to default PLINK settings (0 or 1 copies of the minor allele), but an alternative coding to 0 vs. 2 copies resulted in no qualitative changes in conclusions or in identification of the most significant SNPs on chromosome X.

To further investigate and refine the significance threshold, the entire association testing procedures for GMS and CGS, GMS and CGS F508del/F508del were performed for each of 1,000 permutations, with genotypes permuted relative to the phenotypes and covariates. Although PCs are derived from genotypes, they represent patient-specific ancestry, and thus remained aligned with the phenotype. From this pool of study sample permutations, results were randomly drawn to obtain 10,000 null permutations for each of the meta-analyses. The obtained significance thresholds for a genome-wide error rate of 0.05 were P=1.07×10-7 (GMS and CGS) and P=1.05×10-7 (GMS and CGS F508del/F508del), illustrating the conservativeness of the Bonferroni threshold. Consequently, for either analysis, a P-value of 5×10-8 is sufficient to achieve false positive error control at a genome-wide value of 0.05, even considering the multiple comparisons implied by two separate GWAS analyses.

Association analysis for SNPs in TSS was performed in 1,042 patients using regression as implemented in the variance components framework in Merlin9. Missing genotypes (0.125%) were inferred by Merlin to optimize the power of association8. Additive model regression was conditioned on linkage and covariates for relatedness of patients, sex, and 4 principal components to control for population stratification. Principal component analysis was performed as described (Li et al. Clin Genet. 2010). Association analysis was performed on the 557 TSS F508del/F508del patients in the same manner. Joint analyses of the GMS, CGS and TSS associations proceeded with the meta-analysis approach as described above, with the three studies contributing to the weighted direction-consistent z-statistic.

Combined Conditional Likelihood Approach to Association Testing.

The testing approach described above preserves false positive error control, but it was reasoned that additional power might be achieved by explicit acknowledgement of the GMS sampling design. A case-control approach would artificially dichotomize the data, thus losing power due to variation of the phenotype within the extremes group. An efficient conditional likelihood method for handling extremes of phenotype association data has been described (Huang and Lin, Am J Hum Genet. 2007 March; 80(3): 567-576), but this approach requires sampling within a predefined region of the phenotype (e.g. precise tails). However, use of this approach was not possible for the data, as the GMS study employed initial entry criteria which had only an approximate (though strong) correspondence to the Consortium lung phenotype, and the lung function measurements were further refined by retrospective record-based evaluation and subsequent follow-up. To fully exploit these data, a novel but straightforward approach was devised that appropriately conditions on the GMS sampling scheme. The approach uses the assumption that the CGS dataset represents a random population sample, whereas the contribution of the GMS dataset is conditional on the observed phenotypes, where the phenotype selection criteria are completely unspecified. The SNP genotypes g were recorded as the number of minor alleles at the locus, and common lung phenotypes y in each study were pre-adjusted for sex and the study-specific PCs described above. A population additive association model y=β₀+β₁g+ε,ε˜N(0,σ²) was assumed. The full likelihood conditioned on the GMS phenotype sampling was

$$\begin{matrix}L = p ( {g}_{CGS} , {y}_{CGS} ; {β}_{0} , {β}_{1} , {σ}^{2} ) \\ p ( {g}_{GMS} \vert {y}_{GMS} ; {β}_{0} , {β}_{1} , {σ}^{2} ) \\ = p ( {g}_{CGS} ) p ( {g}_{GMS} ) \\ p ( {y}_{CGS} \vert {g}_{CGS} ; {β}_{0} , {β}_{1} , {σ}^{2} ) \\ p ( {y}_{GMS} \vert {g}_{CGS} ; {β}_{0} , {β}_{1} , {σ}^{2} ) \text{/} \\ p ( {y}_{GMS} ; {β}_{0} , {β}_{1} , {σ}^{2} ) ,\end{matrix}$$

where

$$p ( {y}_{GMS} ; {β}_{0} , {β}_{1} , {σ}^{2} ) = {\sum }_{j = 0}^{2} p ( {g}_{GMS} = j ) p ( {y}_{GMS} \vert {g}_{GMS} = j ; {β}_{0} {β}_{1} , {σ}^{2} ) .$$

Finally, for each SNP the statistic 2 (_(log-likelihood ratio), with the null likelihood assuming β₁=0. P-values were obtained by comparison of the log-likelihood ratio statistic to χ₁² was computed. The approach assumes that the effect sizes are the same in GMS and CGS, which is true under the null hypothesis.

Power Analyses.

Power analyses for the combined GMS and CGS studies were performed by assuming an underlying additive allelic genetic model, with assumed effect) β₁ on the average phenotype for each additional copy of the minor allele (as described in the additive model immediately above). Only the results for GMS+CGS F508del/F508del are shown in FIG. 4. The power derived from all patients is higher due to the greater sample size. Using the CGS sample to estimate the population mean and variance and assuming the underlying effect size, the posterior probabilities of the genotypes were computed using the actual observed phenotypes (for both GMS and CGS), and they were used to simulate accompanying genotypes. In this manner, the simulated data respected the GMS extremes-of-phenotype design and any exaggeration of apparent effect sizes due to the design. Then, for each simulation, the weighted meta-analysis procedure was performed, with the meta-analysis P-value compared to the genome-wide threshold 5×10⁻⁸.

SNP Genotype Imputation.

Using the hidden Markov model algorithm implemented in MACH (available online at sph.umich.edu/csg/abecasis/mach/) and IMPUTE (available online at /mathgen.stats.ox.ac.uk/impute/impute.html), genotype imputation was conducted for 1162 patients recruited by the University of North Carolina site and 1254 1,254 self-reported “Caucasian” patients recruited by the Toronto site. As some these individuals were later used for the TSS study, association analyses were performed only for the unique subsets in GMS and CGS, respectively, as give in FIG. 2. The reference sample for imputation was the 60 CEU samples from HapMap Phases I and II. For each subject, the genotype for each SNP was reported as a dosage value (a continuous value between 0 and 2), reflecting the expected number of copies of a reference allele at that SNP, conditional on the directly observed genotypes and the phased CEU haplotypes. MACH was used to impute autosomal SNPs and IMPUTE for chromosome X SNPs. Imputation yielded genotype data for ˜2,544,000 autosomal and ˜65,000 chromosome X SNPs, respectively. Approximately 36,000 SNPs with estimated imputation accuracy less than 0.3 (using MACH's r² accuracy measure) were discarded. Refined genotype imputation was conducted for GMS and CGS SNPs on chromosomes 11 and 20, using HapMap Phase III data, thereby increasing the number of novel imputed SNPs by 22,000 across the two chromosomes.

Copy-number analysis. Copy number variations (CNVs) were detected using both pennCNV (2008 Nov. 19 version) and genoCNV (version 1.08) using default parameters. Samples with lower quality were dropped, which were initially identified by relatively larger number of copy number calls and were confirmed by visual inspection. In total 1103 and 1303 samples were used for CNV association in GMS and CGS, respectively. CNVs harboring fewer than 5 probes were filtered out and only the probes with copy number changes in ≧1% of the samples were used in the following association studies, which results in 3,008/4,868 probes from genoCNV/pennCNV in GMS study, and 3015/4663 probes for genoCNV/pennCNV in CGS.

In addition to overall copy number, genoCNV can also identify allele specific copy number. Principal components (PCs) identified from the genotype data were used to account for population stratification, as described for the association study methods. Two models were used to evaluate the association between trait and copy number call, probe by probe. For the j-th probe, the models (i) trait=sex+PCs+cnⱼ; (ii) trait=sex+PCs+cnⱼ+cnⱼ*sex, were considered, where cnⱼ indicates the total copy number of the j-th probe. In addition, genoCNV is able to identify allele-specific copy number. Let acₜ (allele-specific copy number contrast) be the number of B alleles minus the number of A alleles at the j-th probe. Two additional models were considered: (iii) trait=sex+PCs+cnⱼ+acₜ; (iv) trait=sex+PCs+cnⱼ+acₜ+cnⱼ*sex+acⱼ*sex. In addition to overall copy number, genoCNV can also identify allele-specific copy number.

Linkage Marker Selection. 19,566 SNPs were selected from the Illumina 610k SNP array based on minor allele frequency >0.4 and r²<0.01 between adjacent SNPs using Merlin. HapMap 2 (www.hapmap.org) recombination data were used to interpolate physical position (in basepairs) with genetic map (in centimorgans). Average distance between markers was 0.18 cM or 0.13 Mbp. Whenever a physical position did not have match in HapMap, genetic position was estimated assuming uniform recombination rates between adjacent SNPs with established genetic positions. The average information content of the markers was ˜0.9 (multipoint) and ˜0.31 (two-point) throughout the genome as determined by Merlin.

Linkage Analysis.

Linkage analysis for the lung phenotype was performed using the variance components method implemented in Merlin (Multipoint Engine for Rapid Likelihood Inference). Linkage was also performed using SOLAR (Sequential Oligogenic Linkage Analysis Routines). Multipoint IBD probabilities generated by Merlin were used for both linkage programs. Very similar results were obtained when linkage analysis was performed using Merlin or SOLAR. Covariates for linkage were sex or sex and average BMI Z-score. Two-point and multi-point linkage maps were generated with and without covariates. Multipoint logarithm of the odds (LOD) of linkage >2.0 was considered suggestive and LOD>3.7 was considered to be of genome-wide significance.

WFDR And SFDR Methods.

Let P, be the p-value of an association test for SNP i, i=1, . . . , m. FDR control can be achieved by converting the p-values to the corresponding q-values. SNPs with q-values less than the FDR threshold value (e.g. γ=0.05) are declared significant. The expected proportion of false positives among all the positives is then controlled at the γ level. Note that although q-value differs from p-value, there is a monotonic relationship between the two. Thus ranking SNPs by p-value or q-value are equivalent.

Let Zᵢ be the linkage score of SNP i obtained from a previous GWL study using either allele-sharing or parametric approaches. For the SFDR method, m SNPs are divided into K disjoint strata based on the prior linkage information. Without loss of generality, consider K=2 and assign each SNP i to stratum 1 (the high priority group) or stratum 2 (the low priority group) according to whether the linkage score Zᵢ exceeds a threshold C (C=3.3 was used corresponding to significant linkage discussed by Lander and Kruglyak, 1995). FDR control is then applied separately in each stratum at the same γ level (Sun et al., 2006), i.e., q-values are calculated separately for each stratum of SNPs. Ranks of the GWAS SNPs are determined by the corresponding q-values and the original association p-values are used to break any ties among the q-values.

In contrast, WFDR calculates a weighting factor W for each SNP i with weights subject to two constraints: Wᵢ≧0 and W̅=ΣᵢWᵢ/m=1 . The weight Wᵢ is proportional to the linkage signal Zᵢ for SNP i (e.g. Wᵢ=exp(B·Zᵢ)/ν, ν=Σᵢ exp(B·Zᵢ)/m, , , and B=1) (Roeder et al., 2006), and the FDR procedure is applied to the set of weight-adjusted p-values, Pᵢ/Wᵢ, i=1, . . . , m. B=2 was used in the present analysis. The WFDR and SFDR were implemented in a perl program called SFDR, available at http://www.utstat.toronto.edu/sun/Software/SFDR/index.html.

### Example 2

### Modifier Loci of Cystic Fibrosis Related Liver Disease

Genetic Variation that Modifies Clinical Phenotype in CF (Liver Disease)

As described herein, a GWAS study of 294 CF patients with CFLD and 1,837 CF patients without CFLD was used to identify a genetic locus on chr 21q22.3 that likely causes severe CF liver disease (CFLD) through non-CFTR genetic variation. The 6 SNPs in the 3′ end of SLC19A1 (rs914232; rs2330183; rs2838956; rs1051266; rs4819130; 30 rs3788190) and 4 SNPs in the 3′ end of COL18A1/endostatin (rs2236483; rs2838950; rs3753019; rs12483377) strongly associate with CFLD.

### Mechanism of Liver Disease Pathogenesis in CF.

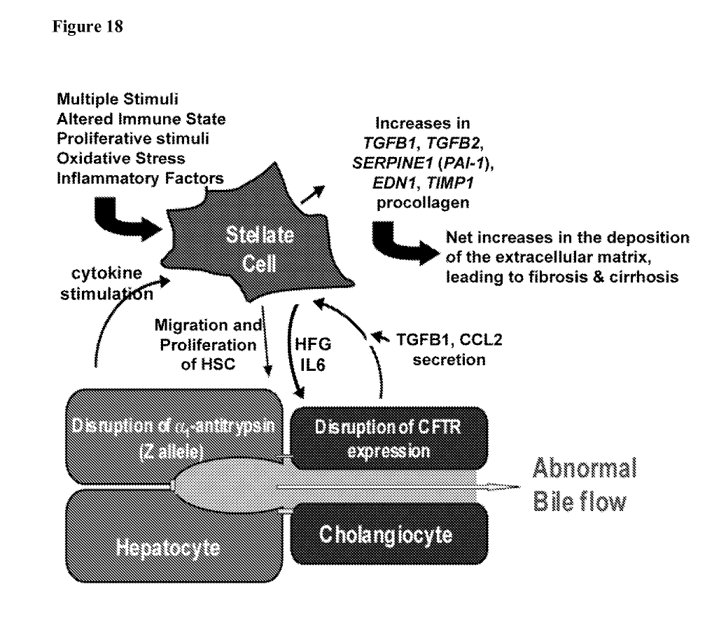

Only 5-7% of CF patients develop severe liver disease, even though most CF patients have abnormal hepatic biochemical markers, changes in tissue architecture on biopsy, or evidence of HSC activation. In CF, signaling molecules are released from activated cholangiocytes, in response to abnormal (sluggish) bile flow, which initiates stellate cell activation and proliferation, and leads to increased collagen production and fibrosis (FIG. 18). Since most CF patients have some evidence of liver disease, there appears to be a state of susceptibility that leads to more significant cirrhosis if additional stimuli are operative, which is congruent with the recent report that the Z-allele is a strong risk factor (OR ˜5) for the development of CFLD (overall p ˜9×10⁻⁹).

### Performing a GWAS.

A GWAS in nearly 300 CFLD patients was pursued. This was done in conjunction with a GWAS in nearly 4,200 CF patients, who were being studied for gene modifiers of CF lung disease by the North American CF Gene Modifier Consortium. The results from this GWAS also provided a special opportunity for CFLD, because there were ample non-CFLD “control” CF patients among those studied for lung disease.

The genotyping was performed on an Illumina 610-Quad platform at Genome Quebec facilities. Extensive measures were taken to ensure quality by adding replicate samples to each plate. In addition, there were extensive datacleaning methods that were undertaken by 2 independent investigators, as well as reclustering and manual analysis of selected SNPs. After data cleaning, 570,725 SNPs from autosomes and the X chromosome were approved for analysis. The patient samples were also scrutinized carefully, and identical by state (IBS) comparisons were used to exclude unexpected duplicate samples or related individuals.

### GWAS Results.

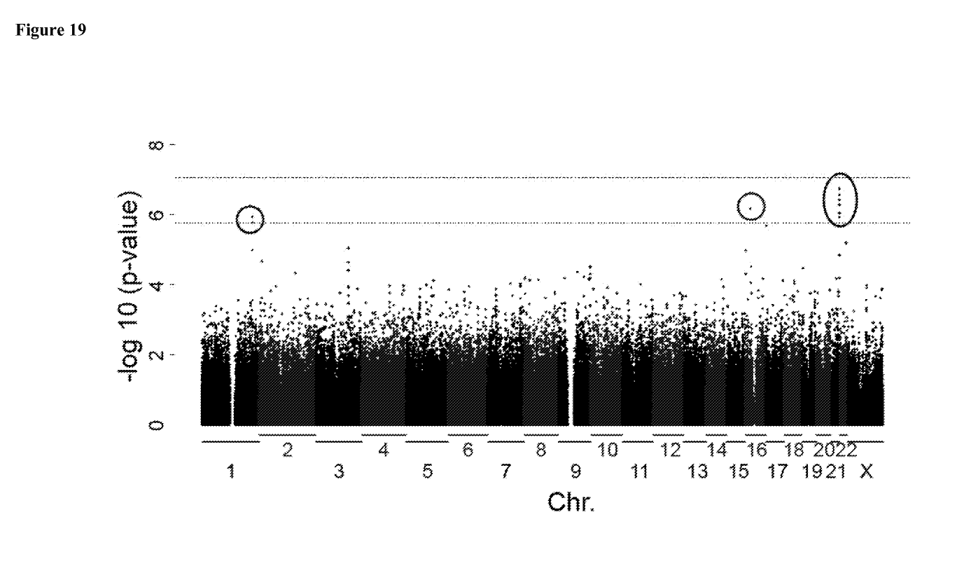

The overall results for the GW AS are shown in a “Manhattan” plot of 294 CFLD patients (184 males and 110 females) vs. 1,837 non-CFLD “control” CF patients (>15 years old) (FIG. 19), based on p-values calculated using a logistic regression model (run with PLINK), and using covariates of gender, principal components (7 “PCs”) and a binary variable indicating whether a patient is homozygous for L1F508 (similar results were obtained when co-variate included whether patient had either one or two copies of L1F508). There are SNPs on Chr. 21 that are near genome-wide significance (p : : : 1×10⁻⁷) for association with CFLD, and the genes in this region (SLC19A1/COL18A1) are discussed herein. There are also SNPs on Chr 1, 16, and 17 that are near or above the “suggestive” threshold for association. The region of Chr 1 is driven by association among males (discussed herein). The SNPs on Chr 16 are near DNAH3 and TMEM159. The latter gene (TMEM159) is also known as promethin, which is one of the genes upregulated in a mouse model of hepatic steatosis. The SNP on Chr 17 (RPA1) codes for a DNA repair protein.

### Chr 21 Region.

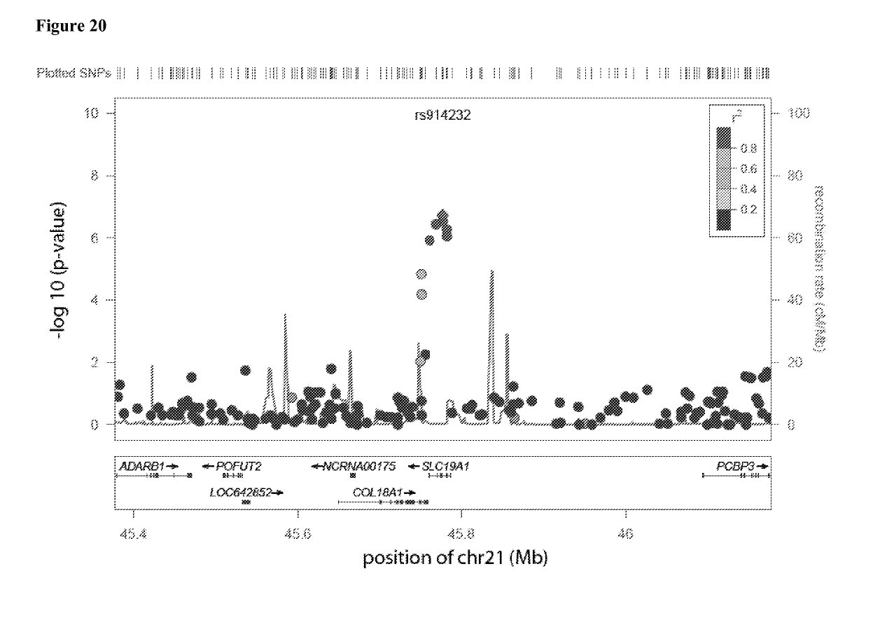

A closer (blown-up) view of the Chr. 21 region (FIG. 20) shows that the SNPs with the strongest association with CFLD are in the 3′ end of two genes: SLC19A1 and COL18A1. SLC19A1 is a folate transporter without a commonly recognized role in liver fibrosis, although moderately high folic acid supplementation exacerbates the development of fibrosis in rats with experimentally CC 14-induced liver fibrosis. Rats that received the folate supplement had 1) higher collagen content, 2) more activated stellate cells, and 3) more apoptotic hepatocytes in liver tissue, compared to controls. In addition, the supplemented rats had alterations in expression of genes related to apoptosis and fibrosis. Thus the highest ranked SNPs in this region exhibited a significant association with differential expression of SLC19A1 (p˜10⁻⁵), i.e., an “eQTL,” in ˜460 samples of LCIs from CF patients. Further, the “risk allele” for these SNPs was associated with increased expression of SLC19A1, in line with the above experimental findings. Therefore, it is likely that genetic variation of SLC19A1 and/or variation in gene expression of SLC19A/contributes to the development of CFLD, i.e., biliary cirrhosis and portal hypertension.

COL18A1 is a basement membrane collagen that is predominantly expressed in highly vascularized organs, such as liver and lung. Whereas no association was seen of SNPs near COL18A1 with lung disease severity, there is a very strong association with CFLD (FIG. 20). There are several reasons that COL18A1 is a strong, biologically plausible candidate to modify fibrosis in the CF liver. First, COL18A1 is expressed (produced) mainly in hepatocytes and activated cholangiocytes and bile duct epithelia of normal and fibrotic livers (Schuppan D, Lancet 1998), which is in striking contrast to the other extracellular matrix proteins, including collagens, which are mainly produced by hepatic stellate cells and myofibroblasts. Second, hepatocyte COL18A1 expression remains constant in acute fibrogenesis (in contrast to greatly upregulated pro collagen a1 and TIMP1), while it is upregulated in regions of biliary fibrosis and cirrhosis (Schuppan D, Lancet 1998). Third, COL18A1, which carries structural properties of both a collagen and a proteoglycan, is upregulated during angiogenesis and early extracellular matrix remodeling, and COL18A1 has a role in binding other basement membrane proteins and proteoglycans, as well as cell surface receptors, playing a role in basement membrane reorganization and angiogenesis (Marneros A G, Olson B R, FASEB J 2005). Finally, the C-terminal domain of COL18A1 is proteolytically cleaved to release a 20K peptide, endostatin, which exhibits potent antiangiogenic effects and for which functionally relevant genetic polymorphisms exist.

Endostatin induces apoptosis in endothelial cells, and has potent antiangiogenic activity. The liver's response to injury involves angiogenesis sinusoidal remodeling, and pericyte (i.e., HSP) expansion. Thus, genes related to angiogenesis may be important modifiers of liver fibrogenesis, including COL18A1/endostatin. Animal experiments suggest that anti-angiogenic agents (such as VEGF inhibitors) might provide an antifibrotic approach, but complete inhibition of angiogenesis might limit hepatic blood flow, with adverse consequences, especially in biliary fibrosis (Patsenker E, Hepatology 2009). There is a delicate balance between angiogenesis vs. anti-angiogenesis in response to liver injury/repair, while the role of COL18A1/endostatin in hepatic fibrosis remains to be examined. Loss of function mutations in COL18A1 in humans and experimental animals lead to vitreoretinal degeneration and hydrocephalus, but no overt liver disease; but deletion of the COL18A1 gene leads to enhanced arterial and cardiac angiogenesis, and delayed dermal would healing (Li Q and Olsen B R, Am J Pathol 2004; Moulton K S, Circulation 2004; Seppinen L, Matrix Bio/2008). The pathophysiology in CFLD could involve both the loss of CFTR function and variant function of COL18A1 and/or endostatin. The apoptotic (proliferative) effects of endostatin (collagen XVIII) are thought to be selective for endothelial cells, but its (and intact collagen XVIII's) effects on apoptosis (and proliferation) of hepatocytes, cholangiocytes, and hepatic stellate cells merits further investigation in hepatocytes and cholangiocytes.

### Example 3

### Modifier Loci of Meconium Ileus

### Determination of Gene Modifiers of MI

### Genotyping and Quality Control

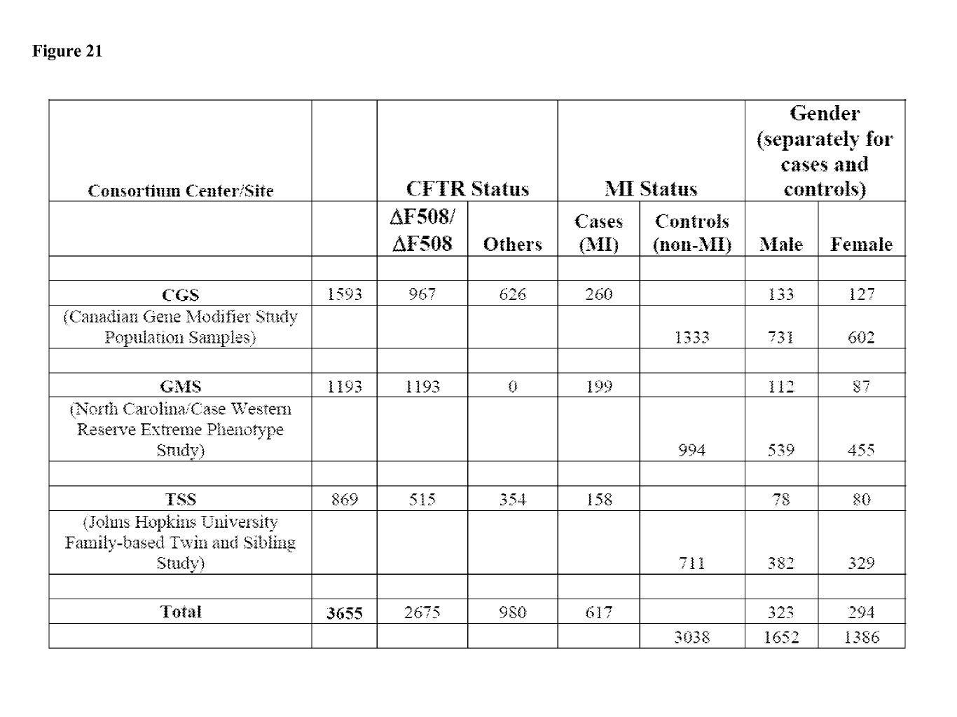

As part of the North American CF Consortium, all CF patients at Genome Quebec (McGill University and Genome Quebec Innovation Centre, Montreal) have been genotyped. Illumina BeadStudio software was used to call the SNP genotypes across the entire set and quality control conducted concurrently. After quality control, samples were analyzed for lung function modifiers. Subsequently, the samples from each site were pooled for a meta-analysis of lung function. A total of 3655 Caucasian patients (ethnicity determined via principal component analysis), analyzed at 556,445 autosomal SNPs and 14,280 chromosome X SNPs, were included in MI modifier analysis; FIG. 21 summarizes the consortium samples used in the MI GWAS, by demographics. Cryptic relatedness across all study samples was determined using identity by decent statistics calculated using PLINK and PREST-plus, with five first-degree relations previously unknown, identified.

### GWAS Analysis and Genome-Wide Significant Results

Given the presence of the subset of related samples from the TSS collection and the identified cryptic relatedness, Generalized Estimating Equations (GEE) was used with an exchangeable correlation matrix to assess the evidence for association between MI and each SNP in the total sample of n=3655 CF patients, adjusting for ascertainment site. Genotypes of the autosomal SNPs were coded additively as 0, 1 and 2 minor alleles, and the X chromosome SNPs were coded 0, 1 and 2 for females and 0 and 2 for males. In parallel, the subset of unrelated samples (n=3055) was also analyzed using logistic regression, adjusting for site and 7 principal components estimated using eigenstrat to assess the evidence for population stratification. Adjustment for principal components had little effect on the results and was deemed unnecessary. The two analyses, logistic regression with PCs and GEE without PCs provided very similar association results with no evidence of stratification.

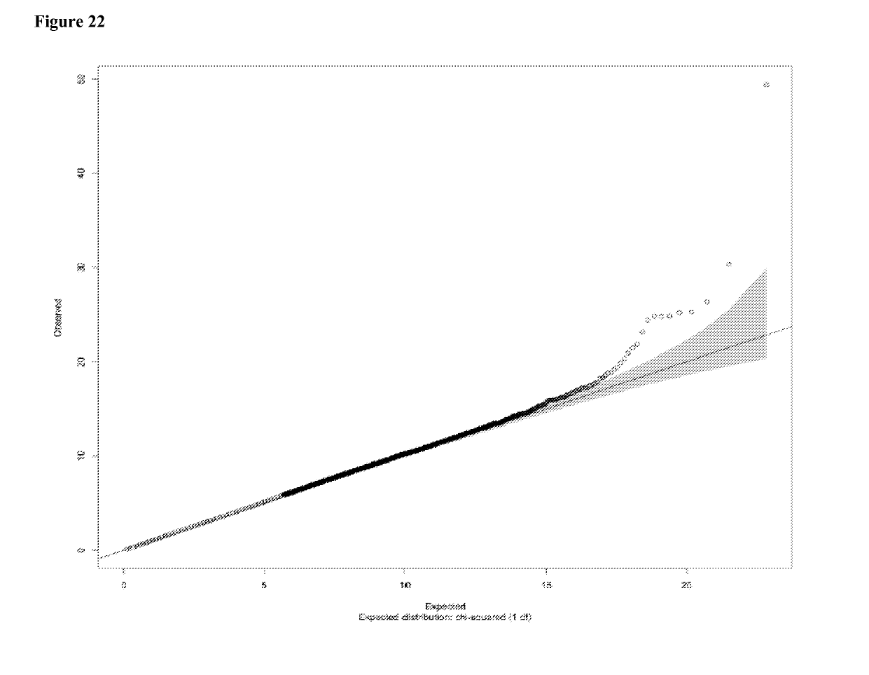

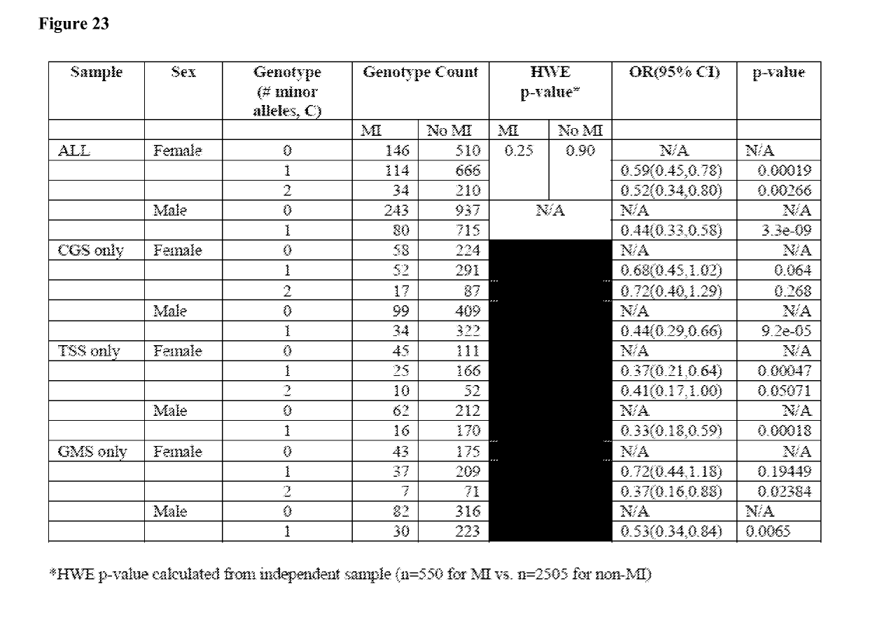

FIG. 22 provides a QQ-plot of the GEE analysis (n=3655) indicating significant departure from random (or chance) findings. The most significant set of SNPs are located on the X chromosome, in the promoter region of SLC6A14, a gene known to encode a protein at the intestinal brush border. To interpret association results from the X chromosome, multiple factors such as how male and female genotypes were coded for analysis, X-inactivation status, sex-specific odds ratios (OR), and Hardy-Weinberg equilibrium in females are also considered. rs3788766 (minor allele frequency (MAF)=0.41) provided the smallest p-value (2.09×10⁻¹²), exceeding the strict genome-wide significant threshold (p<5×10⁻⁸). The rs3788766 normalized intensity plot was as expected for an X chromosome SNP. When analyzed separately by males (p=3.3×10⁻⁹) and females (p=1.4×10⁻⁴, calculated from an additive model) both sexes contribute association evidence, all three ascertainment sites contribute to the association, and a single C allele is sufficient to decrease one's MI risk, under the assumption of X-inactivation; OR=0.44 (95% CI 0.29-0.66) in males and OR(CC vs TT)=0.52 (95% CI 0.34-0.78) and OR(CT vs. TT)=0.59(95% CI 0.45-0.78) in females (FIG. 23).

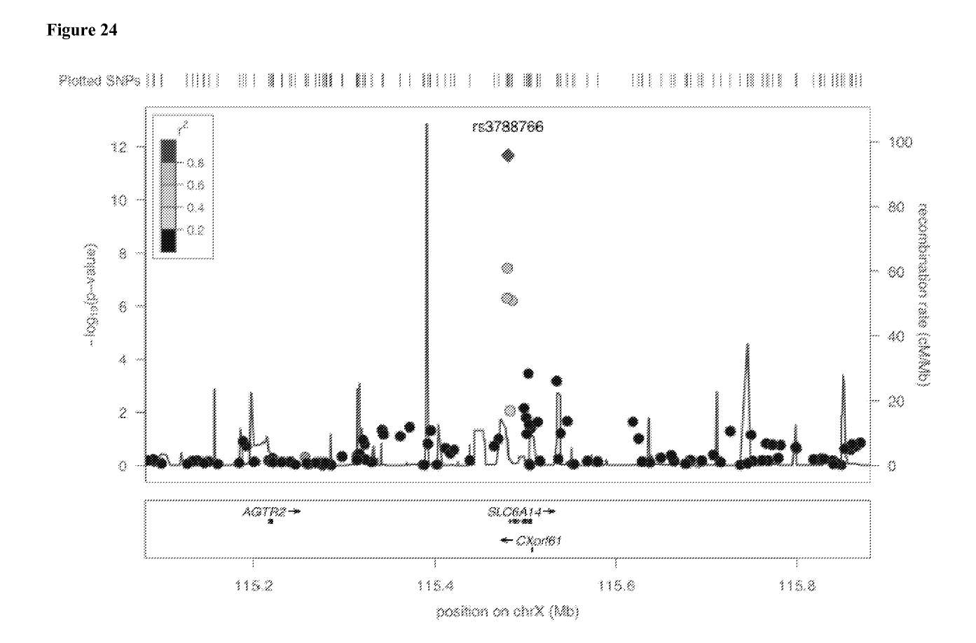

The regional plot (FIG. 24) suggests that all SNPs associated in SLC6A14 are in linkage disequilibrium (LD) with rs3788766 (measured by r²). Conditional analysis confirmed the absence of allelic heterogeneity. CNVs in the SLC6A14 region have not been reported in the database of genomic variants (see projects.tcag.ca/variation/).

Adjustment for CFTR mutation, genome-wide, had little effect on the results indicating that the genotypic associations are not confounded by CFTR mutation, including those that are known to lead to appropriately localized, but dysfunctional CFTR protein (e.g. CFTRG551D) as well as misfolded CFTRΔF508 that is retained in the ER and rapidly degraded. However, due to low frequencies of patients carrying mutations other than ΔF508, there is little power to detect interactions with CFTR.

### GWAS-HD and Suggestive Association Results

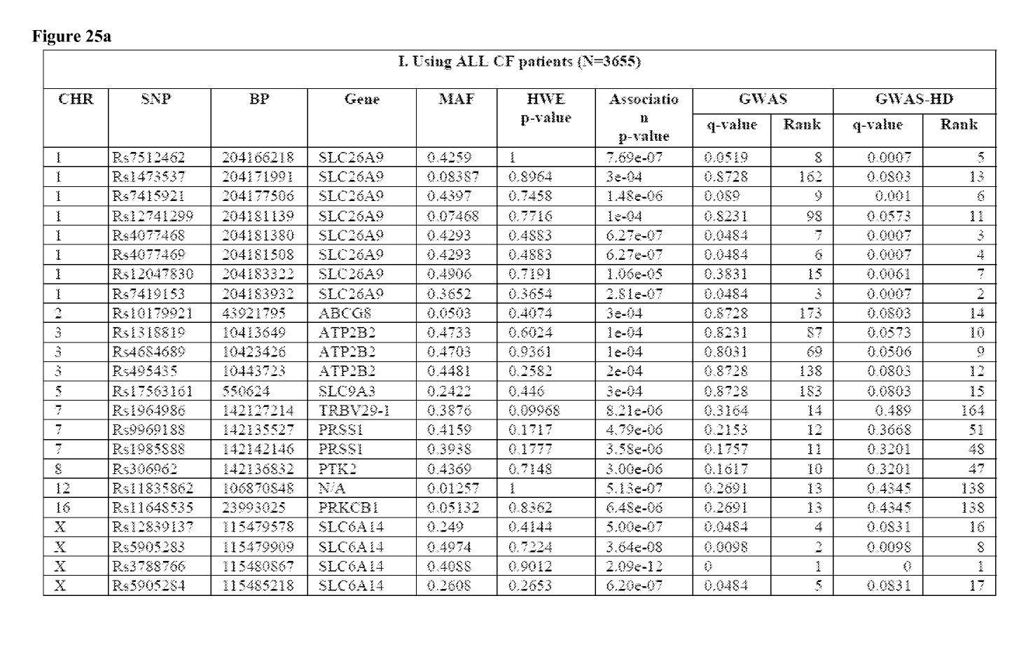

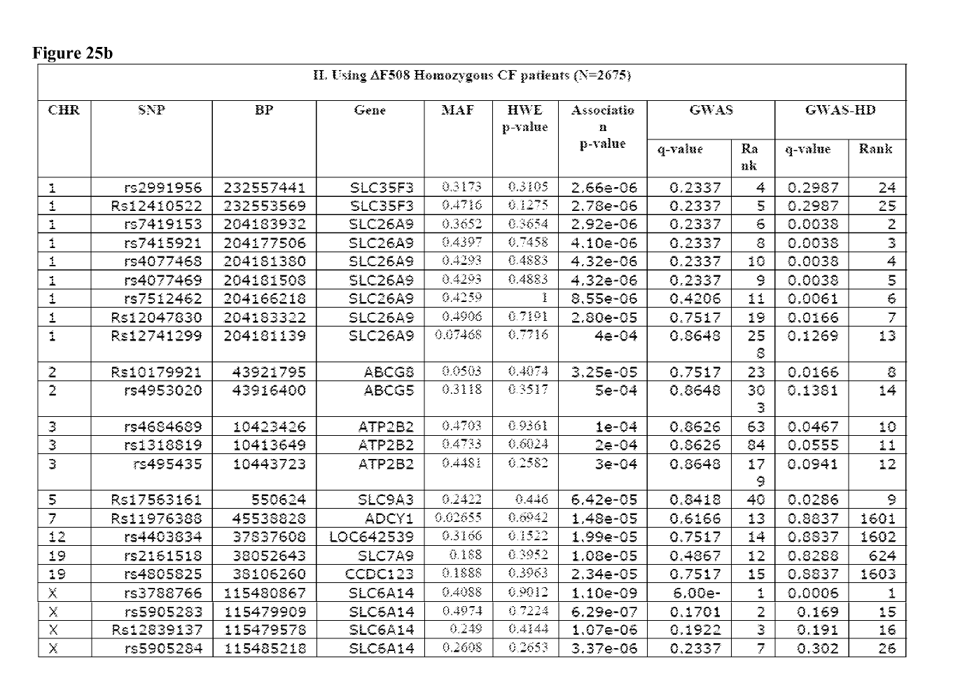

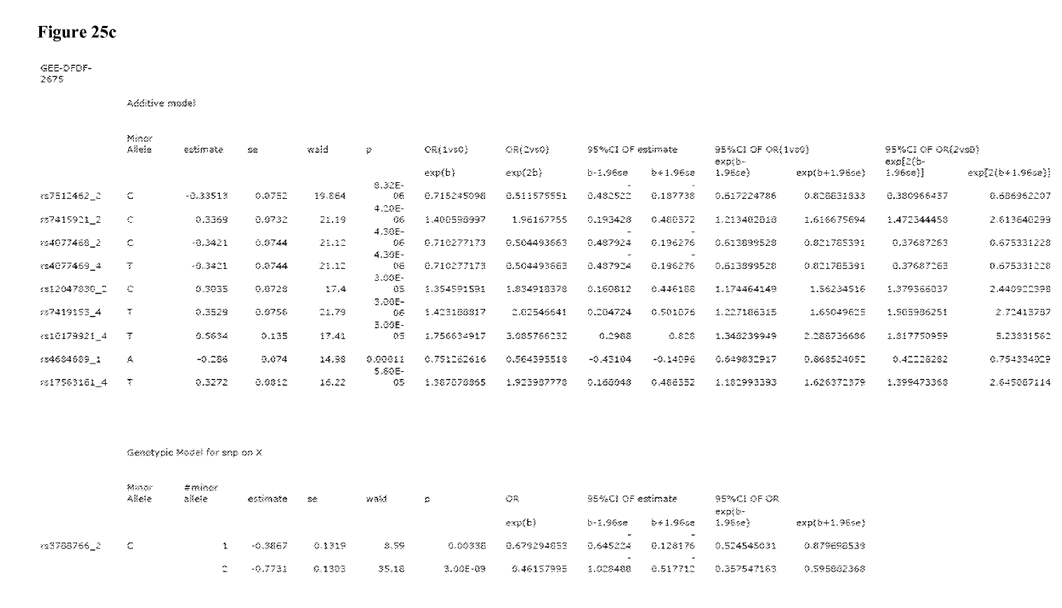

Although SLC6A14 contains SNPs that reach a strict genome-wide significance criterion, these particular SNPs cannot explain all the MI genotypic variance (pseudo R²=0.027), and there are likely other genes which contribute to MI. The GWAS results indicate a number of SNPs that provide suggestive association evidence (p<10⁻⁵) (FIG. 25a—using all 3655 CF patients, and FIG. 25b-restricted to 2675 ΔF508 homozygous CF patients), including those in SLC26A9, a gene member of the SLC 26 family that encodes a protein in the apical plasma membrane of the gut lining and other organs. MAF and HWE p-value were calculated based on 2505 independent controls (no-MI). The p-value was obtained from association analyses using all available samples adjusting for a center/site covariate, via the GEE model that accounts for the correlation between a subset of samples that are related. The q-value is genome-wide adjusted p-value that controls the False Discovery Rate. The rank is the rank of a SNP at the genome-wide level based on its association p-value alone (GWAS) or combined association evidence incorporating the apical plasma membrane hypothesis (GWAS-HD). FIG. 25c provides OR estimates for all SNPs in FIG. 25a with a q-value <0.05, as well as identifying the minor allele.

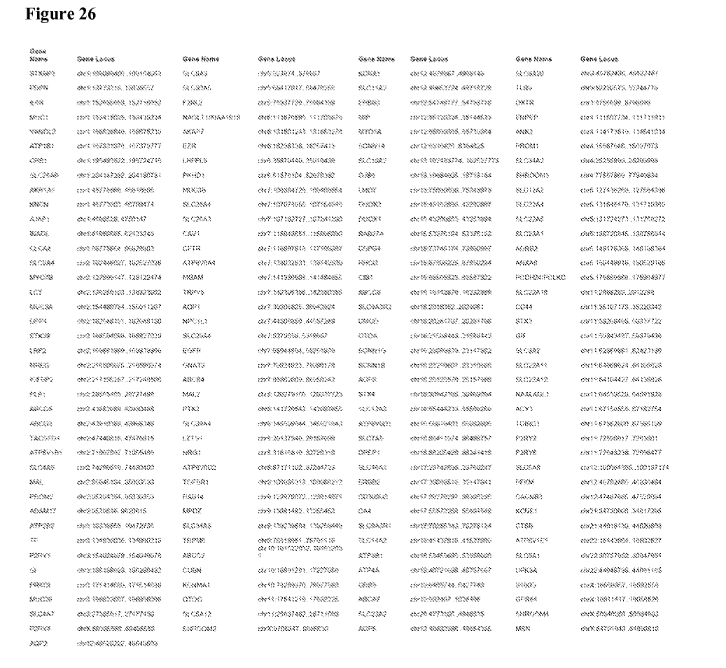

Given that loss of CFTR function leads to perturbed epithelial transport, it was considered that coincident phenotypes including MI reflect residual or adapted transport capability. Given the distinct localization of many transport-relevant proteins in polarized epithelial tissue, it was hypothesized that constituents of the apical plasma membrane, where CFTR normally resides, may be contributors to the MI phenotype. To test this hypothesis in the MI GWAS data, the Hypothesis-driven GWAS (GWAS-HD) methodology was developed and applied by: 1) prioritizing the genome by generating a list of genes that encode proteins that localize to the apical plasma membrane (as set out in FIG. 26—this list of 157 genes was annotated by AmiGO version 1.7 (downloaded Mar. 28, 2010), based on GO consortium, generated by location search phrase apical plasma membrane with restriction to homo sapiens). and assigning GWAS SNPs to a high priority group (SNPs within the boundaries of these genes) or a low priority group (all others); 2) using the stratified FDR approach (SFDR) to weight GWAS p-values and determine genome-wide significance for given loci; and 3) performing permutation testing to determine the statistical significance of the hypothesis.

The apical plasma membrane list consisted of 151 genes spanning 3,723 GWAS SNPs, although eight genes were not tagged by any of the ˜550K GWAS SNPs. SNPs were assigned to genes if they were within ±10 kb of the gene boundaries as annotated from public databases. Although CFTR and many solute transporters are included, SLC6A14 is not on this gene list despite it being on the apical brush border membrane, likely reflecting the high specialization of this type of intestinal cavity and a limitation of the GO annotation that was accepted without additional curation.

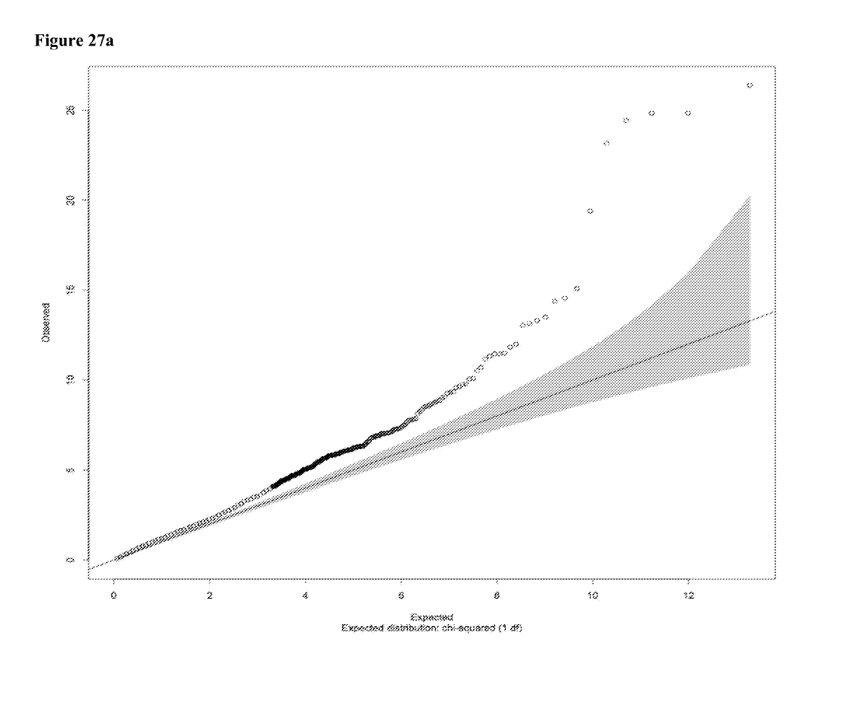

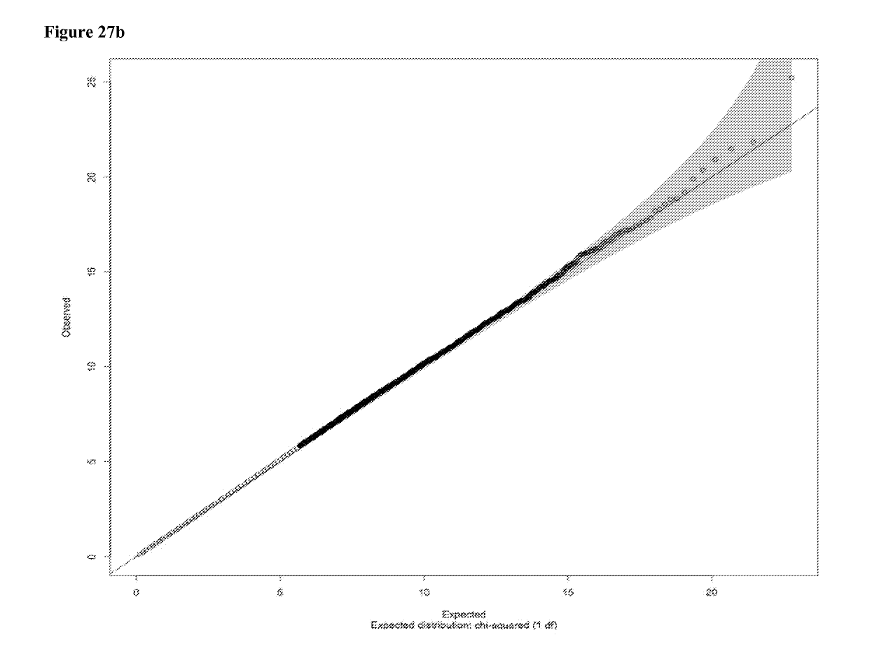

FIG. 27a provides the qq-plot of the 3,723 SNPs in the apical membrane list, whereas FIG. 3B provides the qq-plot of all of the remaining SNPs. Note that in this first analysis, X chromosome SNPs were omitted to exclude SNPs reaching genome-wide significance. Clearly there is substantial deviation in the observed p-values from expected in FIG. 27a in contrast to FIG. 27b. The deviation appears to begin early in FIG. 27a, at approximately a chi-square value of 5 (corresponding to p-value of 0.025), and this deviation consists of 175 SNPs spanning 40 different genes from the original list of 143 genes. However, since independence is one of the underlying assumptions in this plot, it is not clear that the deviation reflected in the figure is significant and that there is truly a preponderance of apical plasma membrane constituents that can contribute to MI susceptibility. As part of GWAS-HD this departure was formally tested (accounting for LD correlation between SNPs in a gene), as well as determined the significance of individual SNPs after prioritization.

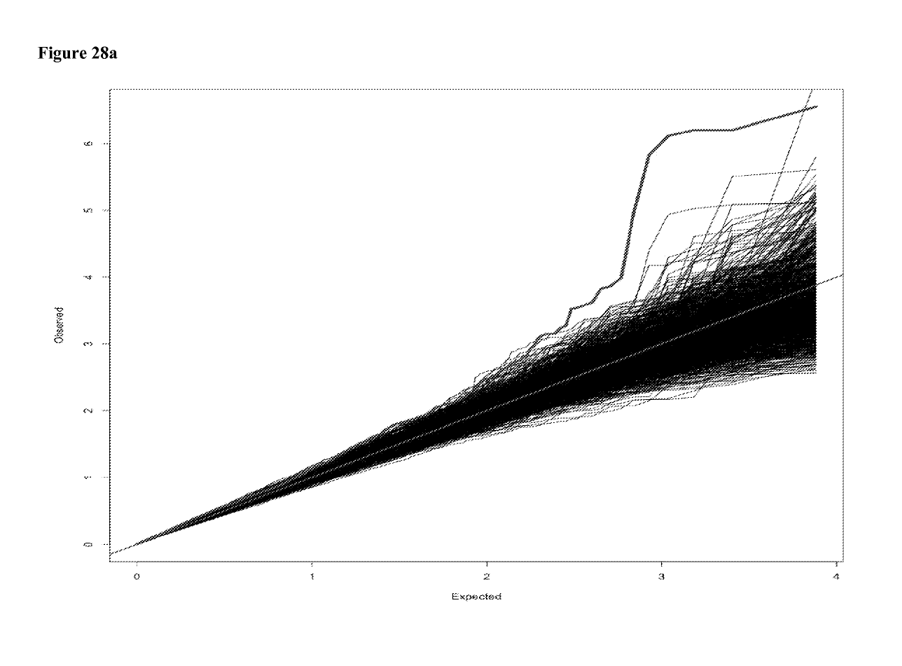

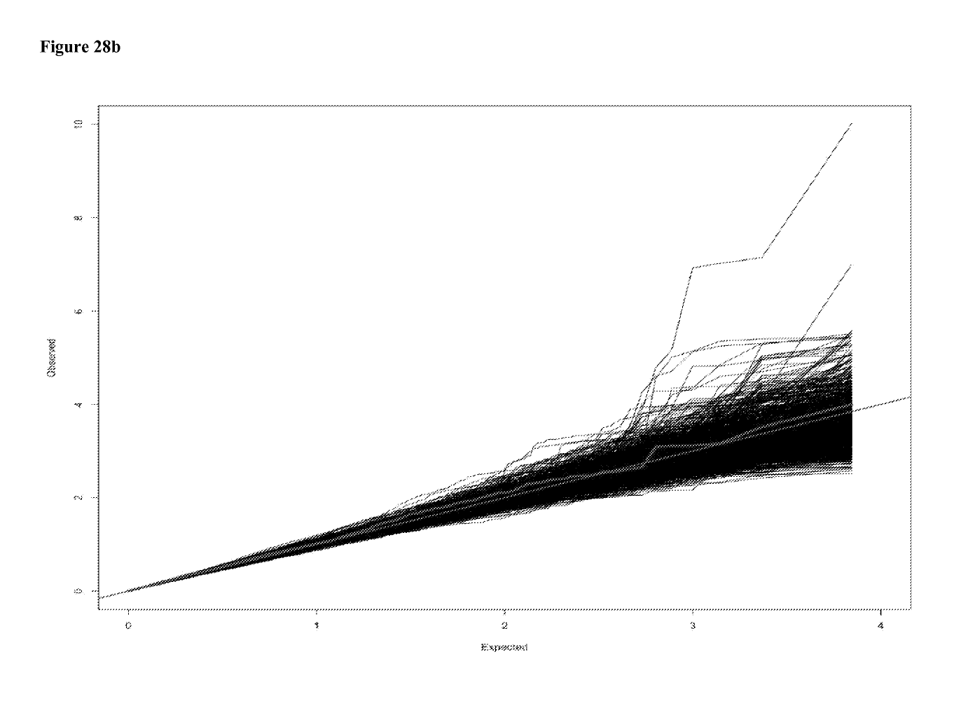

To test the hypothesis, the MI case control phenotype status was permuted 1000 times to obtain 1000 null simulated datasets that retain the same LD pattern of markers as in the apical plasma membrane gene list. The association evidence was then reanalyzed across the 143 genes of interest for each of the 1000 permuted replicates. The qq-plot in FIG. 28a displays the distribution of p-values across these 1000 null datasets (black curves). The red curve is the observed result, which is clearly an extreme as compared to the null distribution of black lines (p<0.001 or 0 out of 1000 permuted datasets are more extreme than the observed one, calculated using a Shapiro-Wilks test); this implies that multiple genes on the apical plasma membrane are associated with MI. For comparison, a null hypothesis/list of membrane-localized genes was constructed for which no relationship with MI was expected. This list consisted of all gene products present in the nuclear envelope also defined by GO annotation, involving 231 genes. FIG. 28b provides the observed and permuted qq-plots associated with this list, and as expected, there was no significant relationship between multiple gene products of the nuclear envelope and MI (p=0.95).

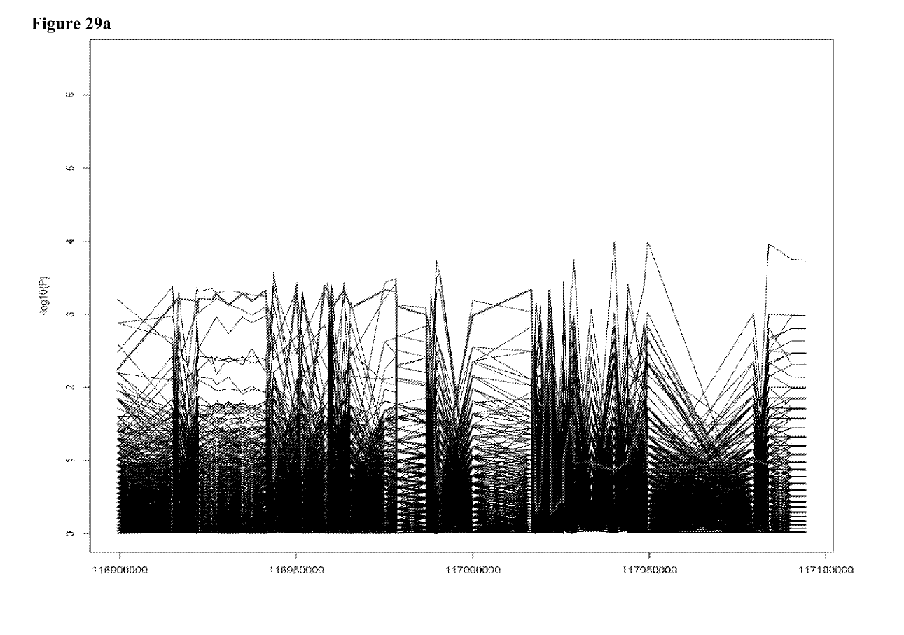

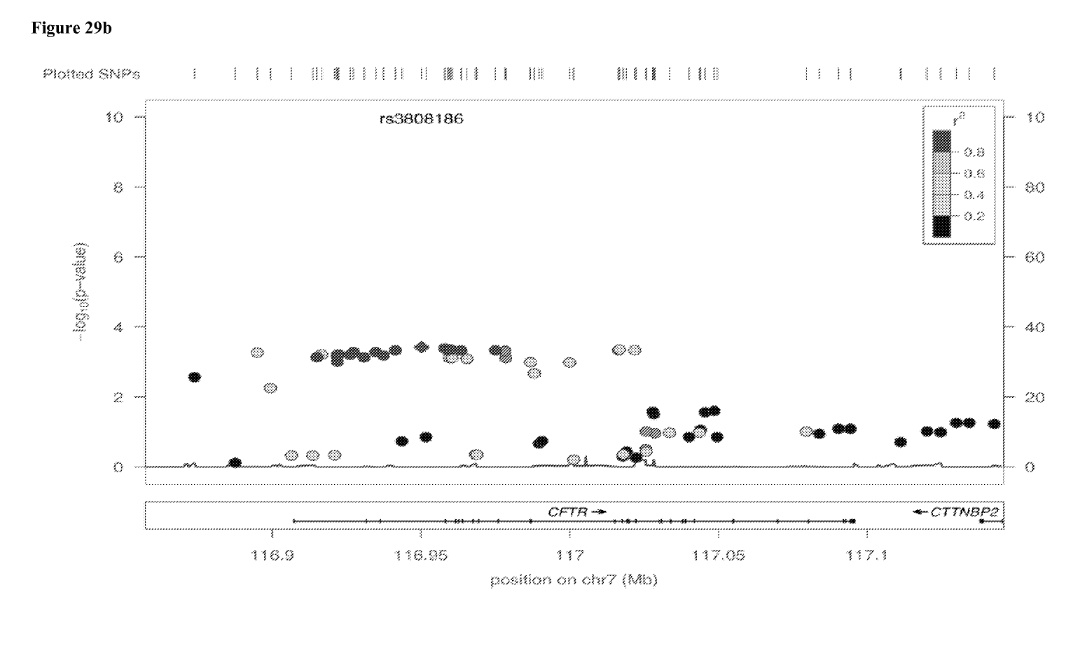

Permutation analysis was also used to assess significance because the genes in the lists are not summarized by a single SNP (e.g. with the minimum or median p-value), but rather including the p-values from all SNPs genotyped in a given gene. While complicating the qq-plots (e.g. FIG. 27) by what can be substantial LD, keeping all typed SNPs in the analysis ensures that susceptibility variants on different risk haplotypes are included. CFTR, itself, is present on the apical membrane list and emphasizes the benefit of retaining multiple associated SNPs. FIG. 29a provides the gene-based perspective, where the red line connects the p-values observed in CFTR and each black line represents the p-values connected for one of 1000 simulated replicates in CFTR (simulated assuming no association between MI and CFTR). The red line in FIG. 5A is an extreme of the distribution of black lines, despite only moderate individual p-values spanning CFTR (FIG. 29b). When each SNP-MI association was adjusted by CFTR mutation type (i.e. using broad categories of mutations that lead to immature protein, truncated protein product or aberrant transcripts) all association with MI in CFTR disappears i.e. the new observed red line would sit at the bottom of the distribution of black lines, highlighting that there is substantial allelic heterogeneity represented by SNPs tagging different CFTR mutations. Thus different CFTR mutations provide variable susceptibility to MI, and this MI-CFTR relationship would not be detected by single-SNP analysis on a genome-wide level.

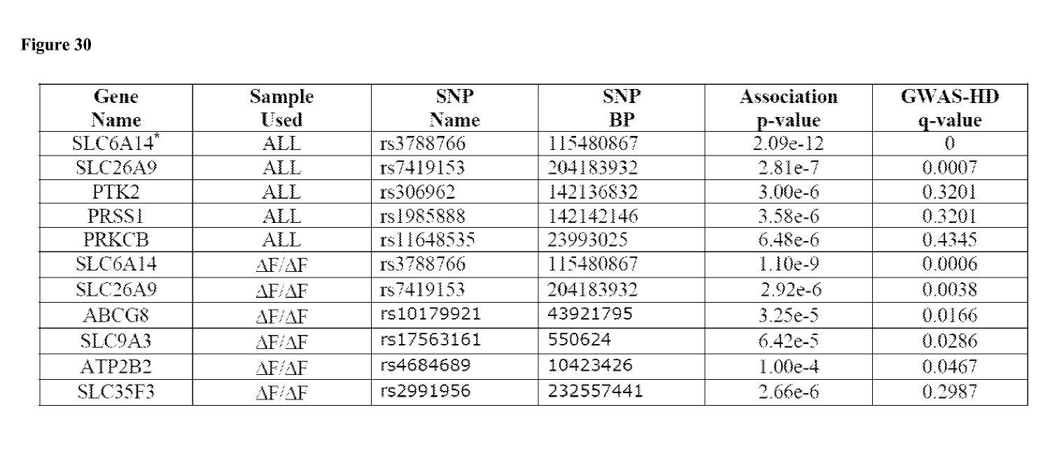

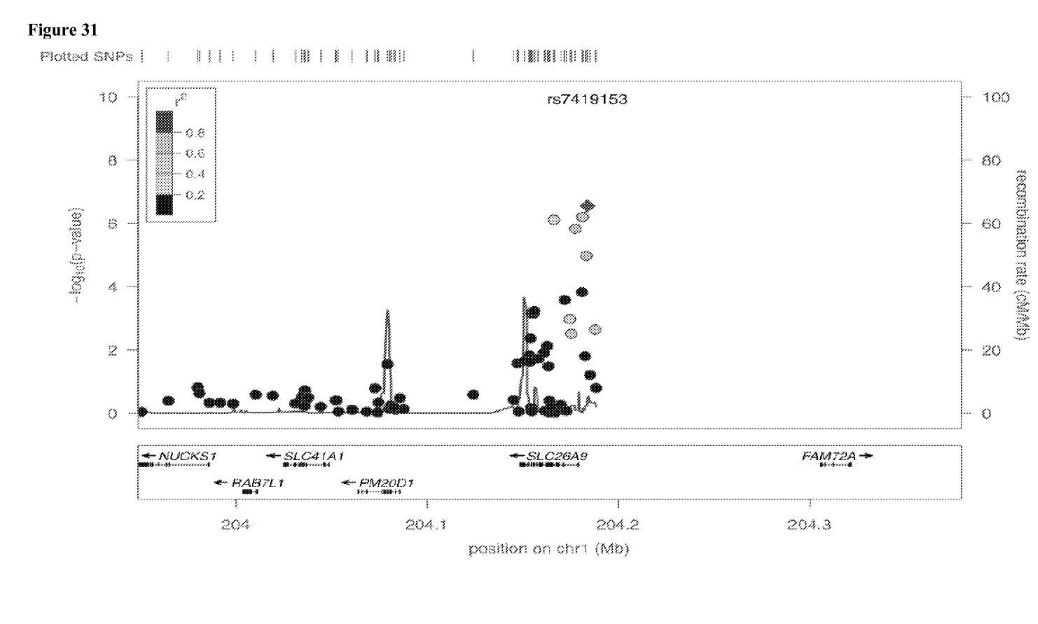

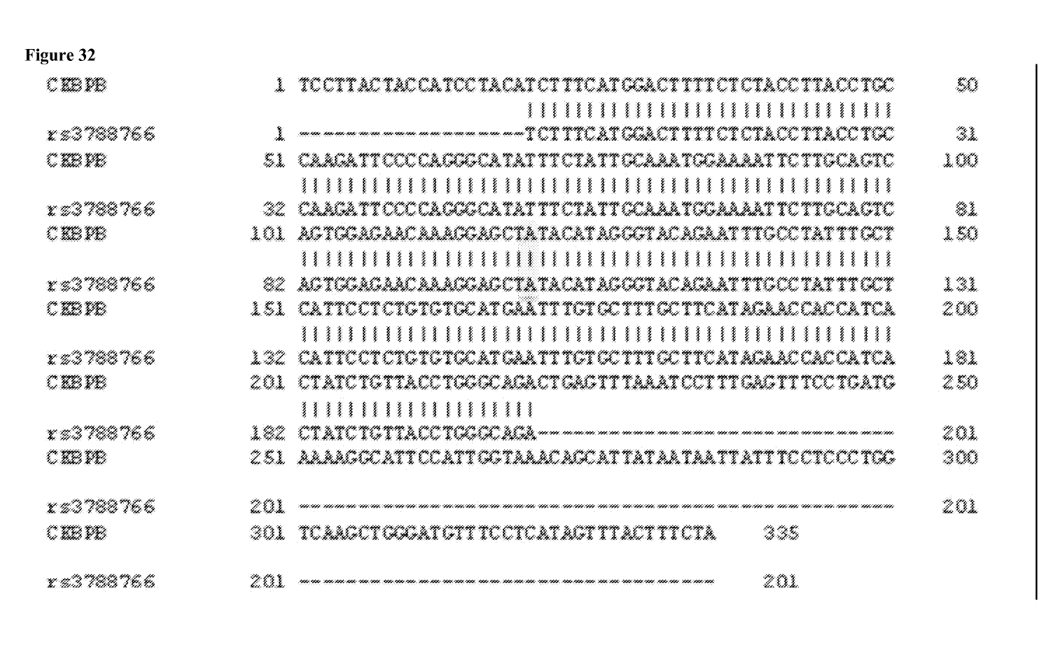

The apical plasma membrane hypothesis was interrogated to determine whether it increased power to detect individual genes that play a role in MI. To accomplish this goal the SFDR (31) was uses as part of the GWAS-HD to reprioritize the genome according to the hypothesis. FIGS. 25 and 30 illustrate that, in addition to SLC6A14, association evidence with MI for gene SLC26A9 increased considerably using the GWAS-HD approach, providing a q-value of 0.0007 when the expected false discovery rate is 0.07% among SNPs with q-value <0.0007. Significance by GWAS-HD of 5 additional SNPs in SLC26A9 (FIG. 25), some of which are not in LD (FIG. 31) was also noted. When analysis is restricted to the subset of individuals who are homozygous for ΔF508, the most common CFTR mutation, multiple SNPs from SLC26A9 remained significant by GWAS-HD (q=0.0038) despite a smaller sample size, as does rs3788766 in SLC6A14 (q=0.0006). The permutation test of the apical membrane list also yielded significance (p=0.002). In addition, SLC9A3, ABCG8, and ATP2B2 from the ΔF508/ΔF508 sub-analysis each provide SFDR q-values <0.05 (FIG. 30).

In summary, the MI GWAS and GWAS-HD provides significant evidence that multiple genes present at the apical plasma membrane may contribute to the MI phenotype. As a result, multiple genes were prioritized for further study, many of which would have otherwise been designated as being of insufficient significance.

### Modifiers for Multiple CF Co-Morbidities

A preliminary inspection of 998 Canadian patients on whom liver disease, diabetes, and MI data were known past the age range of typical onset, suggests a significant number of patients with both MI and CFRD (p=0.04), CFRD and CFLD (p<0.0001), but not MI and CFLD. In addition, evidence for common genetic modifiers between the various co-morbidities has been striking from initial GWAS results. For example, as in the MI GWAS one of the most strongly supported loci in the CFRD GWAS was SLC26A9 (p=5.7×10⁻⁰⁷), with consistency in both the alleles and direction of effect to the MI findings. Although SLC6A14, the strongest MI finding indicated only limited evidence of association with CFRD (p=0.08, rs3788766) and CFLD (p=0.09), some overlap with lung disease findings existed. From the lung disease GWAS analysis, the third most significant locus included AGTR2, the gene neighbouring SLC6A14 on chromosome X. Further, it is notable that some of the SNPs with association evidence at this locus (e.g. rs6520219 with p<1×10⁻⁴) may correspond to the promoter region of SLC6A14. Another interesting observation involves SLC9A3, which is also an apical plasma membrane constituent and is reported to be associated with lung disease in a CF candidate gene study. SNPs in SLC9A3 provided association evidence from both the lung disease GWAS and the MI GWAS (with p=0.0003 for lung and 0.0001 for MI for rs6864158 with the minor allele associated with a decreased risk of MI but with improved lung function, as expected).

### Determination of Modifiers for MI and Lung Disease

A simple linear combination method that uses the average of two phenotype-specific association test statistics accounting for the baseline correlation between the two phenotypes being considered was used to identify loci that influence occurrence of MI and deteriorating lung function, CFRD and CFLD.

The statistic takes the form of (T₁+T₂)/\sqrt{2(1+ρ)}, where ρ is the correlation between the two phenotype-specific association test statistics. This statistic is normally distributed with mean 0 and variance 1 under the null of no association. A preliminary analysis was conducted applying this method to the combination of MI (yes/no) and deteriorating lung-function (quantitative trait as outlined in Taylor et al. appended) to look for common modifiers between the two co-morbidities. An empirical method was used to estimate the correlation value ρ between the two phenotypes by calculating the sample correlation between two vectors of T₁ and T₂ after LD (r²<0.2) and MAF (MAF>0.05) pruning of the GWAS SNPs (94,737 SNPs left from the original 556,445 SNPs). This estimation method is justified by results of both our analytical derivation and simulation studies. The empirical correlation is approximately zero (−0.02), consistent with the observation that having MI does not lead to noticeably poorer lung function. Because it was anticipated that the effects of a SNP on the two phenotypes would be opposing, i.e. if the minor allele of a pleiotropic SNP increases occurrence of MI then it is reasonable that the same allele would correspond to poorer lung function, the above statistic was modified by changing the sign of one of the two phenotypic-specific association statistics.

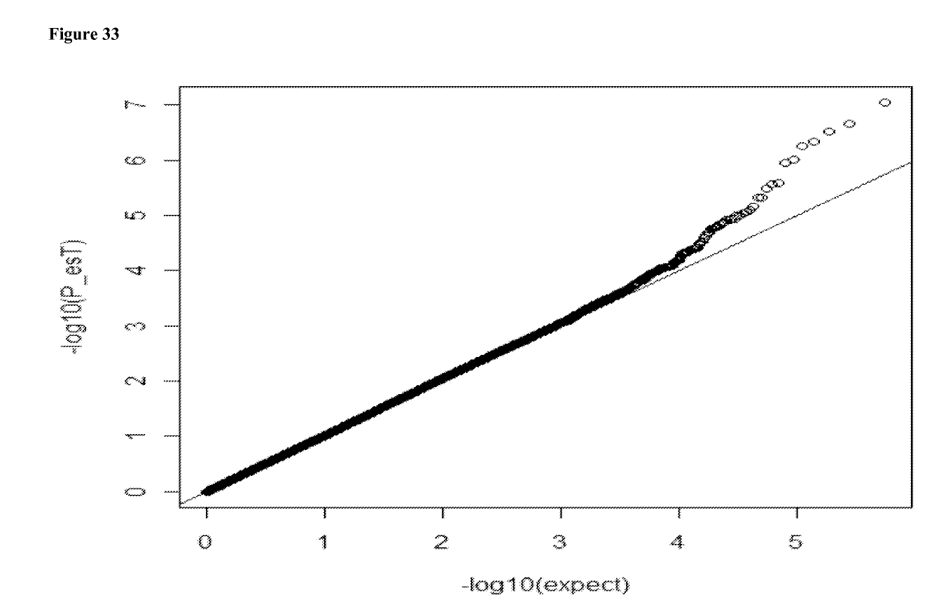

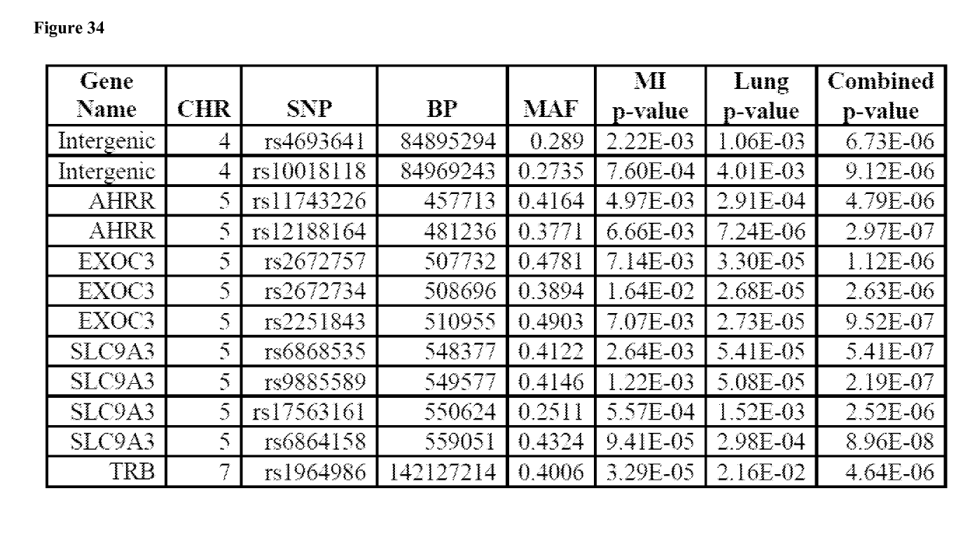

FIG. 33 shows the genome-wide qq-plot of the −log10 (p-values) inferred from the combined statistic for detecting pleiotropic effects. FIG. 34 provides the top 16 SNPs (p-value ≦10⁻⁵) identified by this method. The qq-plot shows that a) the proposed method is accurate in that it does not increase the false positives and b) the tail distribution indicates multiple interesting results (FIG. 34). The top ranked SNPs point us to a gene on chromosome 5, SLC9A3 a member of the solute carrier family present on the apical plasma membrane, and already identified in the MI GWAS-HD AF/AF sub-analysis.

### Functional Assessment of SLC6A14

SLC6A14 has been described as an electrogenic Na⁺ and Cl⁻ dependent amino acid transport system. Therefore, its function and its impact on overall trans-epithelial ion transport can be assessed by means of electrophysiological examination in an Ussing chamber.

Experiments were performed on primary human airway epithelial cells derived from lung explants of CF patients (F508del/F508del). In CF airway cells the dominant ion transport is luminal Na⁺-absorption via luminal Na⁺ channels. After blocking the Na⁺ channels with amiloride, the lumen-negative transepithelial potential difference (mV) decreased significantly. Further, in CF airway epithelial IBMX/Forskolin induced Cl⁻ secretion via CFTR Cl— channels is reduced or absent. Under these conditions, apical application of the amino acid arginine induced an electrogenic dibasic amino acid transport which is characteristic for the SLC6A14 transport system (shown as lumen-negative change in mV). Addition of a specific CFTR inhibitor (CFTR172inh) decreased the lumen-negative potential difference. While addition of CFTR inhibitor did not other alternative Ca²⁺-dependent Cl⁻ channels (normal ATP response), this effect may reflect partial inhibition of the SLC6A14 system.

### Modifiers of Meconium Ileus in Cystic Fibrosis

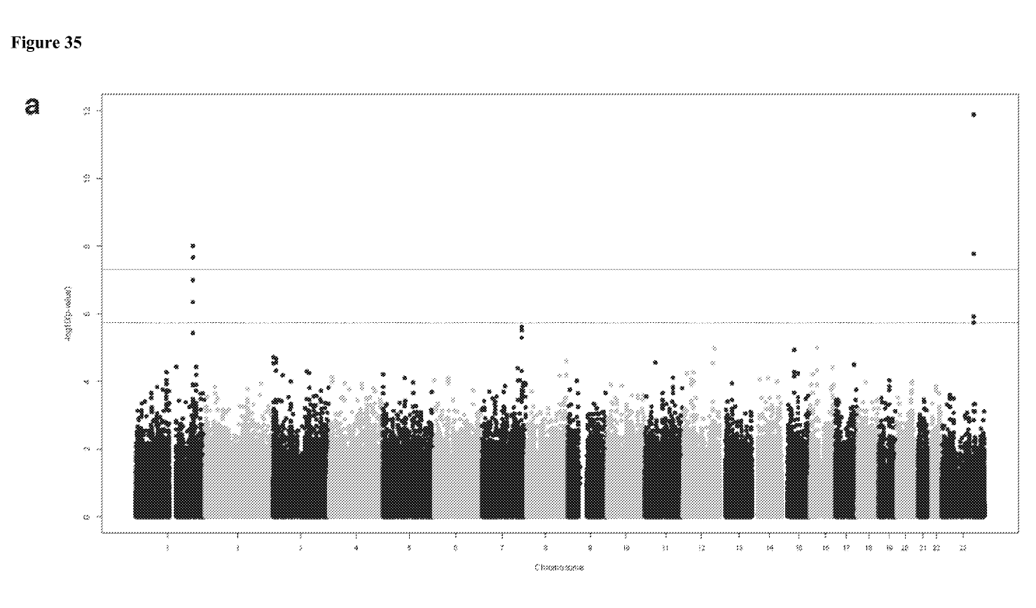

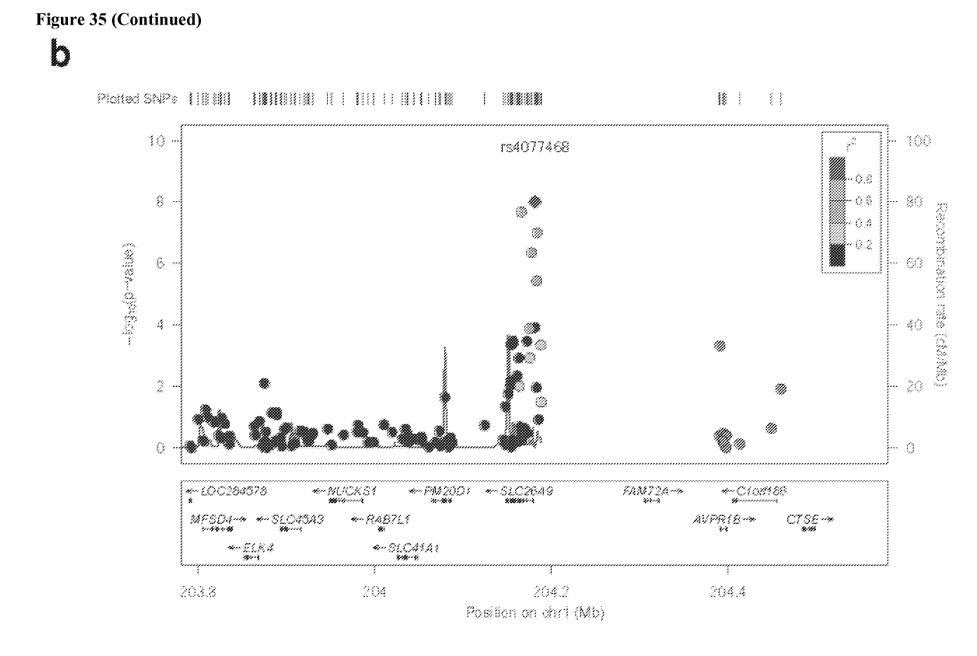

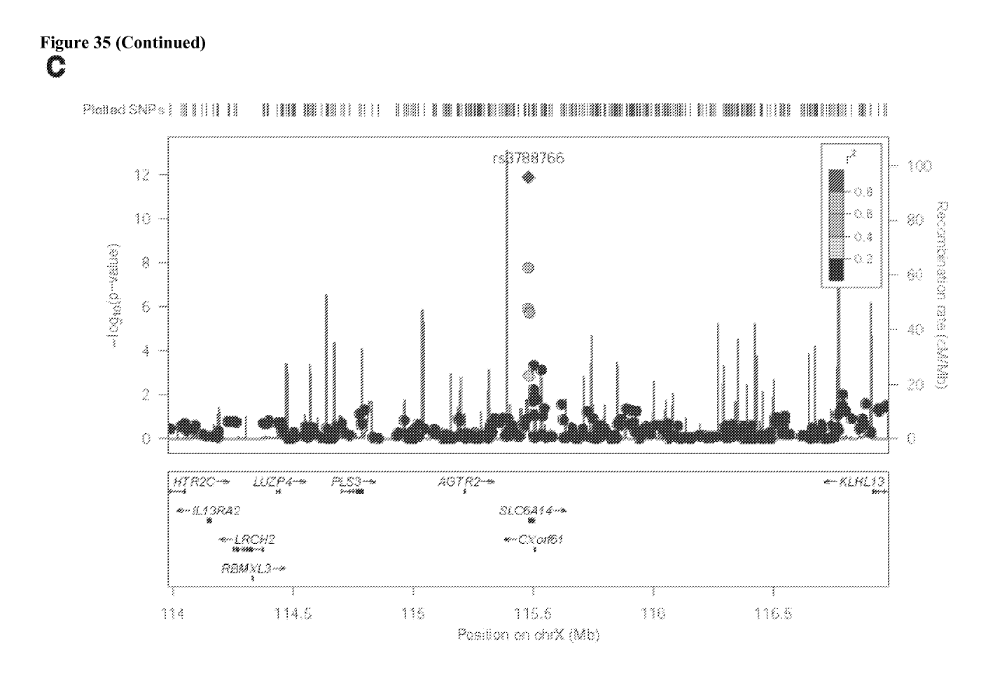

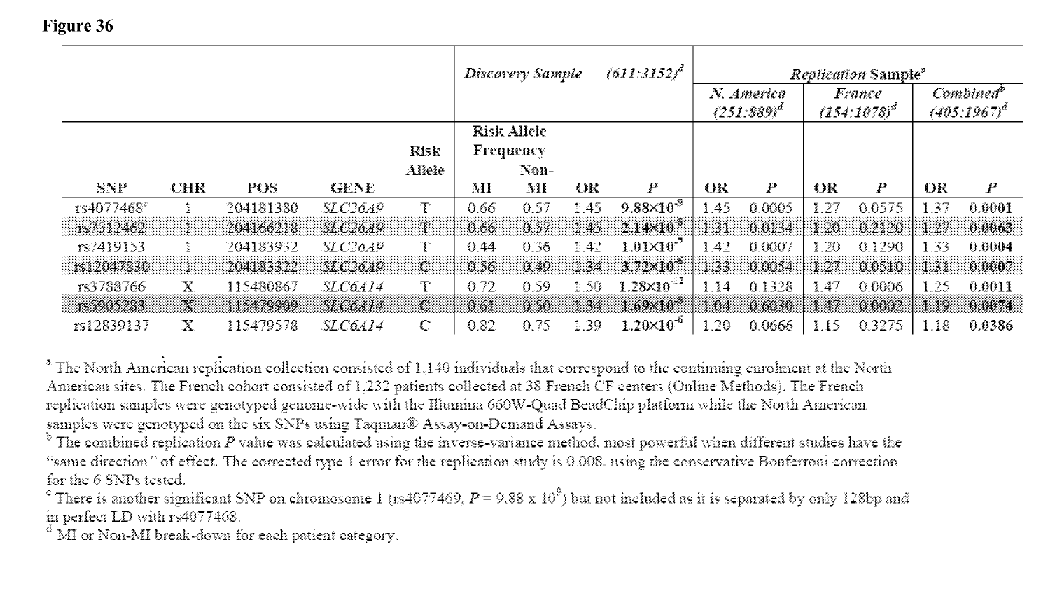

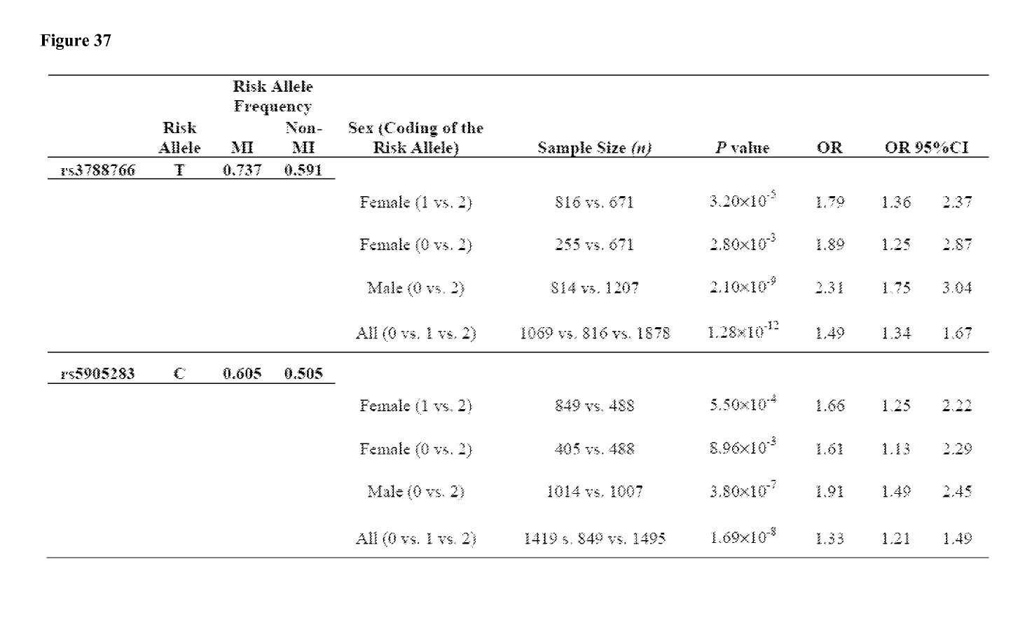

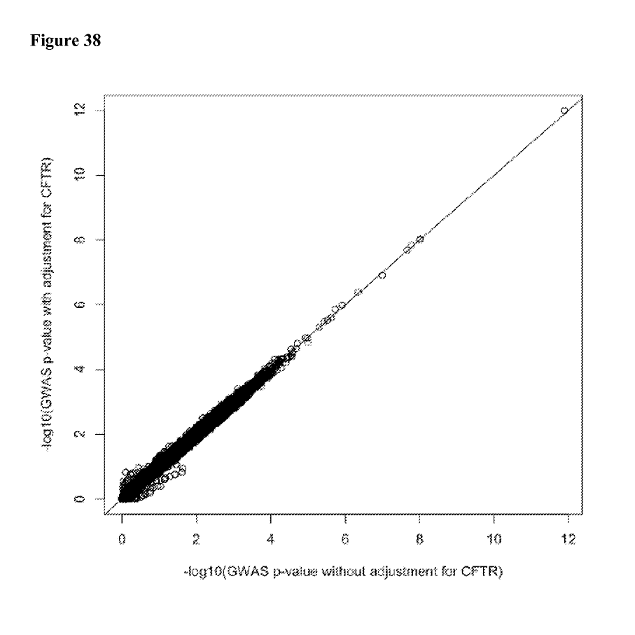

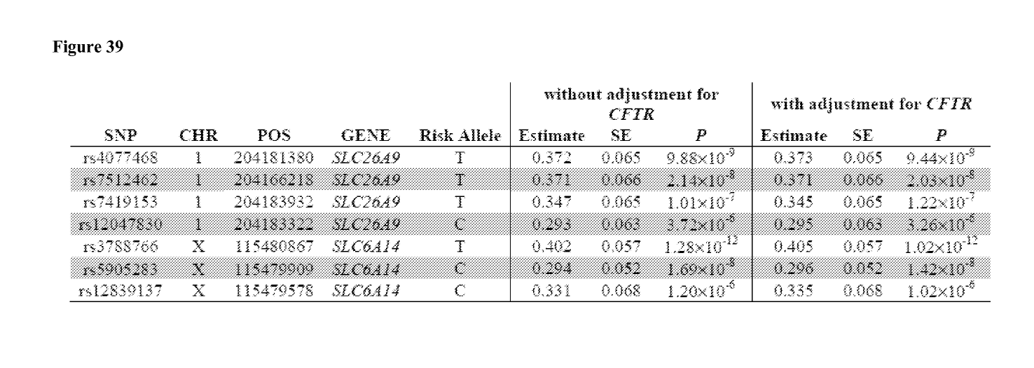

As described above, the North American CF Gene Modifier Consortium has accumulated a large patient collection, with 3,763 participants with ‘severe’ (pancreatic exocrine insufficient) CFTR genotypes and genome-wide genotype data of high quality at 543,927 SNPs. The definition of MI was consistent within the consortium and was recorded following rigorous chart review. The initial GWAS for MI used a generalized estimating equations (GEE) model to include collected sibling-pairs, and led to five genome-wide significant SNPs (P<5×10⁻⁸) from two regions that include SLC26A9 on chromosome 1 and SLC6A14 on chromosome X (FIG. 35, FIG. 36; sex-specific results in FIG. 37). CFTR was not a significant confounder or effect modifier when incorporated in the GWAS (FIG. 38 and FIG. 39), indicating SLC6A14 and SLC26A9 are independent contributors to the MI phenotype.

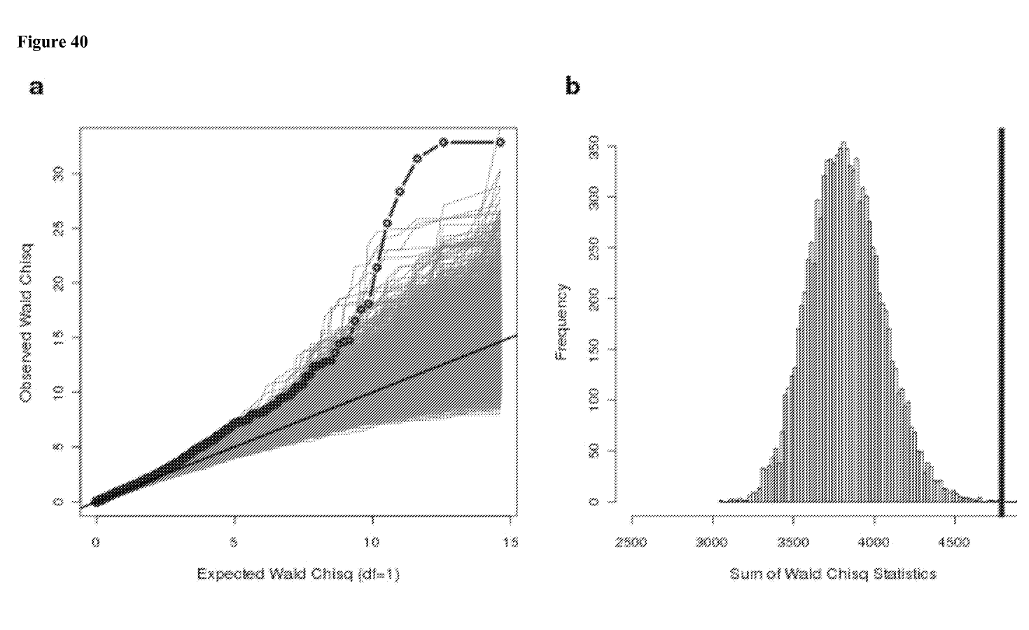

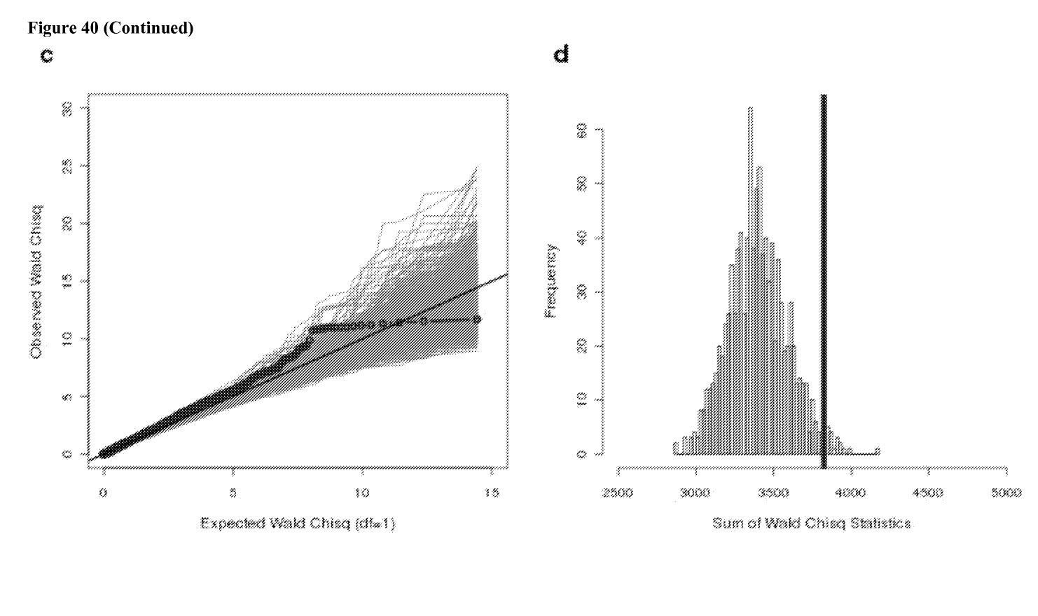

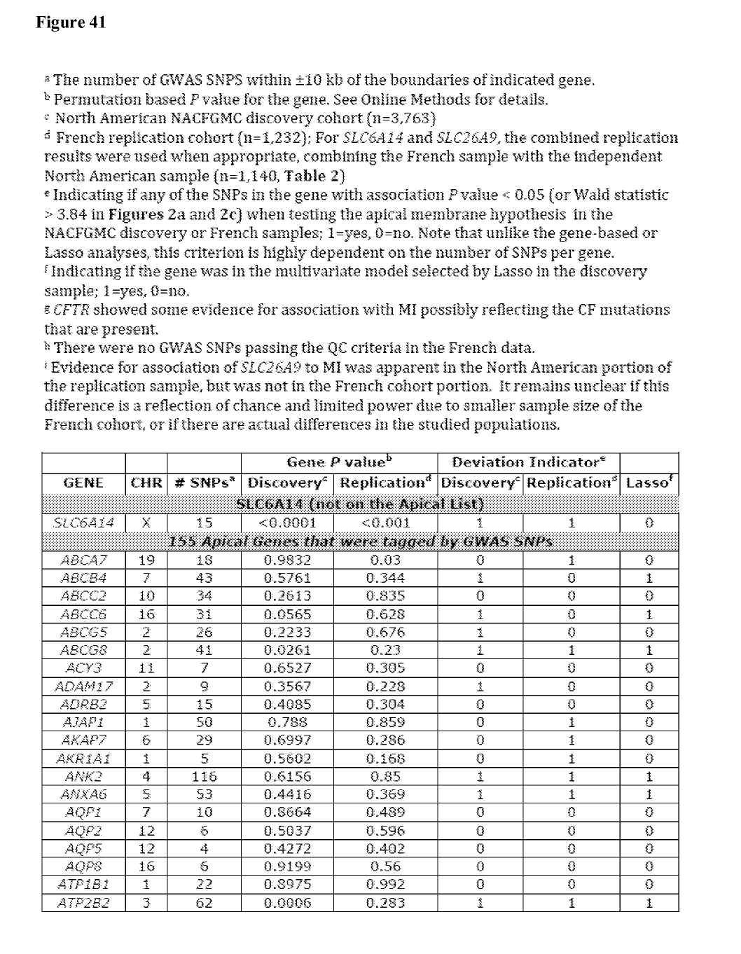

The associations were successfully replicated at SLC6A14 (P=0.001) and SLC26A9 (P=0.0001) with MI in an independent combined sample from the North American collection and a French CF cohort (FIG. 40).

The signal intensity plots of all of the associated SNPs reflected autosomal- and X-associated SNPs at SLC26A9 and SLC6A14, respectively. Imputation analysis using MACH identified the same regions of association as the genotyped SNPs (See Methods). The five associated SNPs for SLC6A14 and two for SLC26A9 are positioned just upstream of their respective transcription start sites such that binding of activating or repressing transcription factors may be affected (FIGS. 35b and 35c) as highlighted by the ENCODE data (data not shown). Neither SLC6A14 nor SLC26A9 coding regions exhibit significant evidence for CNVs; however, there is a noted gap in the genome sequence map >10 kb upstream of the SLC26A9 locus.

The seven SNPs (FIG. 36) of the two replicated genes account for <5% of the MI variation, estimated by pseudo R-squared, likely reflecting substantial locus heterogeneity and low power to detect individual loci or SNPs given the available sample size. However, these genes were identified using the conventional GWAS designed for complex disease mapping, while in modifier gene studies critical disease information regarding the genetic etiology is often available and could be incorporated to identify modifier loci. In this modifier gene study of a recessive genetic disease, there is substantial information about the pathobiology of CF that can potentially lead to identification of a sizable collection of additional associated loci accounting for a much greater proportion of MI heritability. To do so, hypothesis-driven GWAS (GWAS-HD) was used to systematically prioritize the initial GWAS results. The GWAS-HD prioritization is based on the knowledge that a major source of CF pathophysiology is impaired fluid and electrolyte flux at the epithelial interfaces of many CF-affected organs including the airway, intestine, pancreas, liver and vas deferens. In these organs, the polarized epithelial layer forms a highly selective and tight barrier between organ and ductal interfaces. Transepithelial ‘function’ is achieved by cell polarization whereby many determinants and regulators of fluid, solute and ion transport reside at the apical membrane alongside CFTR, with contributing features from basolateral surfaces. In a mouse model it was shown that CFTR function in the gastrointestinal epithelium is critical for preventing intestinal obstructions. Thus, with loss of CFTR, (genetic) variation in other apical membrane constituents could modify CF phenotypes, such as MI.

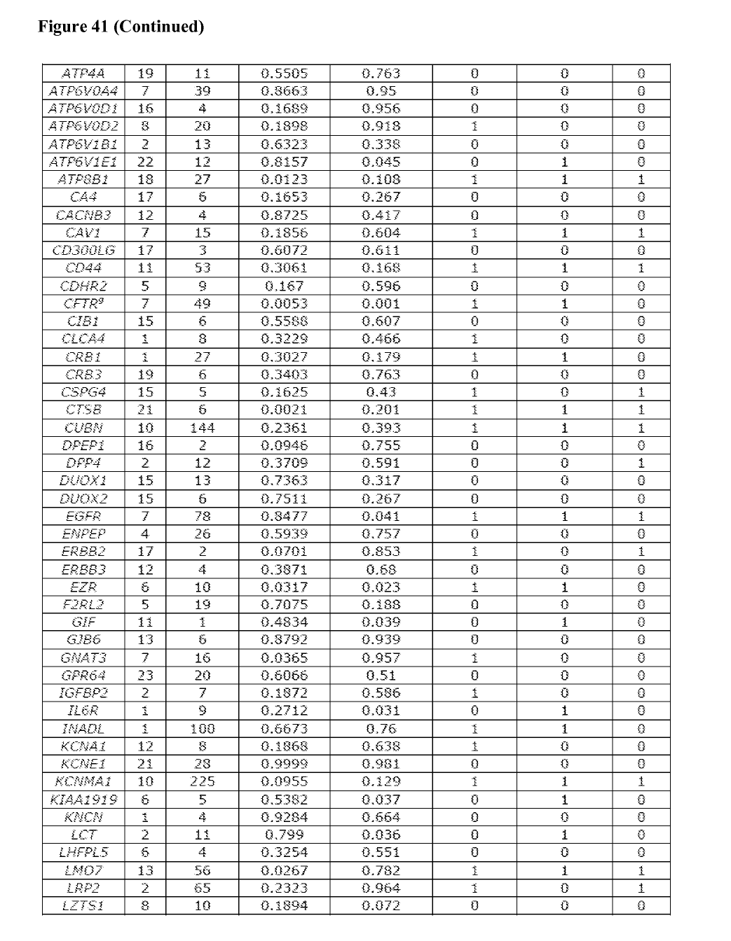

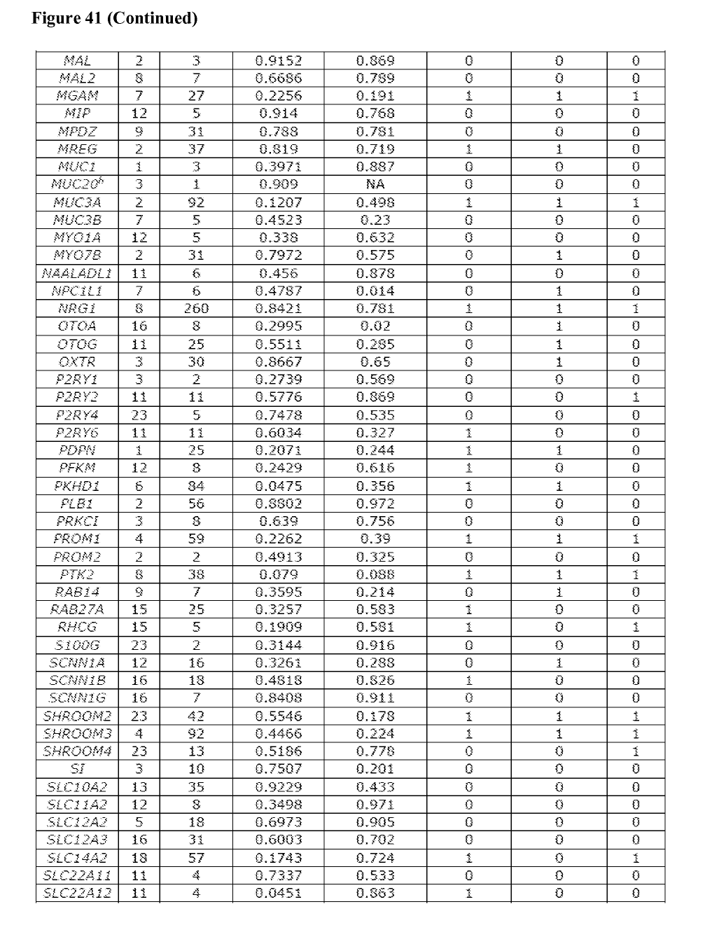

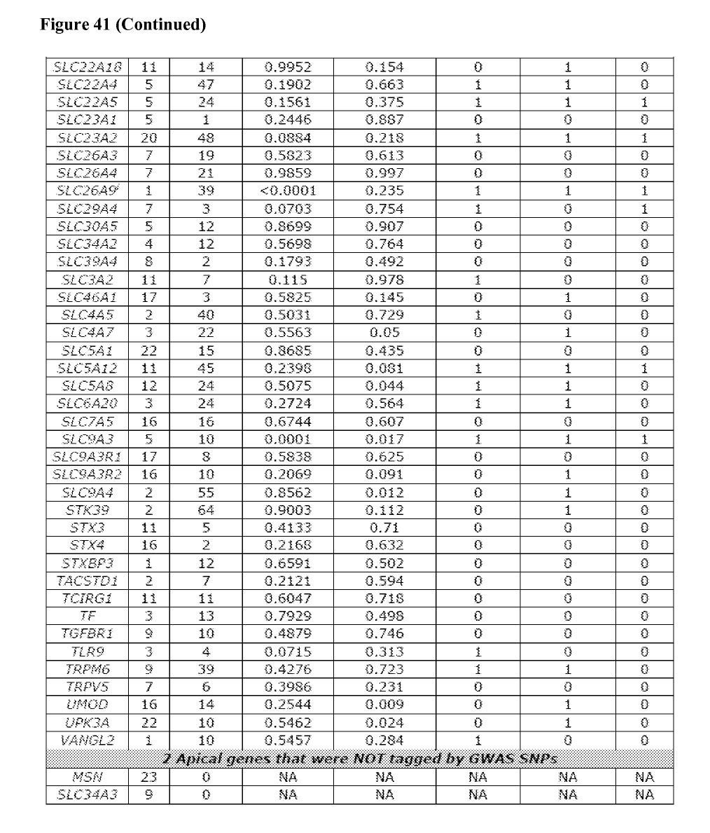

A list of 157 gene products (FIGS. 40 and 41) was annotated as localized to the apical plasma membrane using AmiGO with Gene Ontology data. CFTR and many solute transporters were included. However, the brush border membrane protein SLC6A14 was not listed, reflecting the high specialization of its corresponding intestinal cavity and a limitation of the GO annotation that we accepted without additional curation.

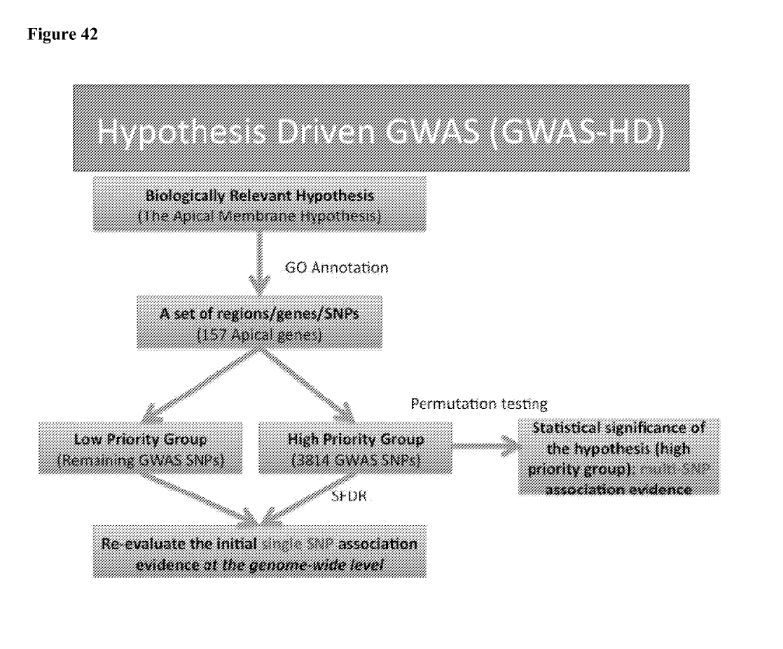

To test the apical hypothesis in the susceptibility to MI, GWAS-HD prioritized the genome by assigning SNPs of the apical genes to a high priority group versus all remaining SNPs of other genes. Two statistical procedures were implemented (FIG. 42). First, using the prioritization, the stratified false discovery rate control (SFDR) was applied to the data to re-evaluate the initial association evidence for any given SNP at the genome-wide level. Then, a permutation-based test was used to determine the statistical significance of the apical hypothesis as a whole, testing all high priority SNPs jointly, to assess whether a preponderance of apical constituents contribute to MI susceptibility.

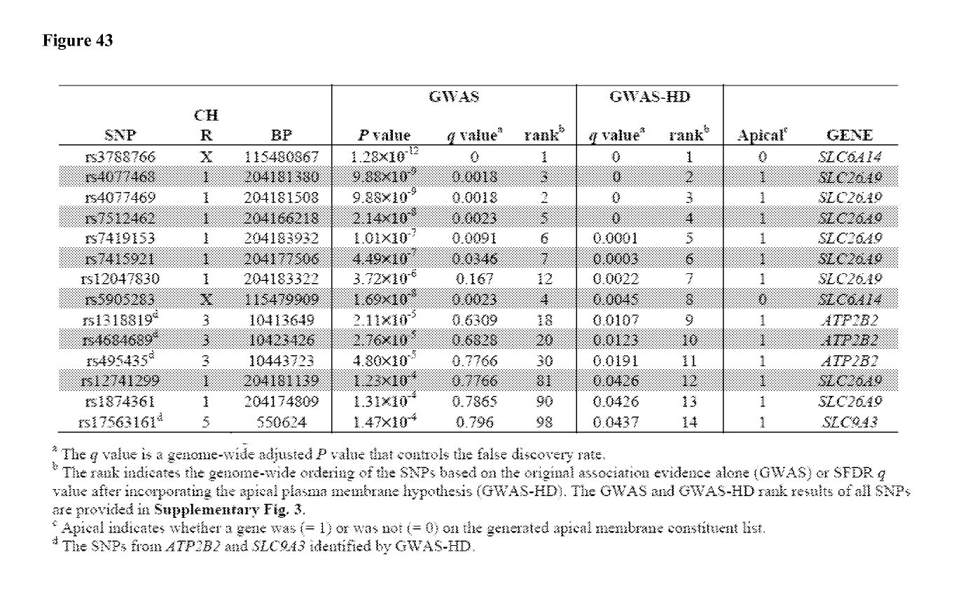

Even after the GWAS-HD analysis, SLC6A14 remained the gene with highest ranked SNPs for association with MI despite being assigned low priority (i.e. not an apical gene), reflecting the robustness of SFDR. In addition to SLC26A9, two genes, ATP2B2 and SLC9A3, showed association evidence with SNPs with q value<0.05 (FIG. 43), indicating that the extra power provided by GWAS-HD was needed to show statistical significance. A gene-based analysis (See Methods) also provided complementary association evidence for the four genes. After Bonferroni correction for the 156 tests performed (155 apical genes tagged by GWAS SNPs and SLC6A14), three genes remained significant, SLC6A14 (permutation P<0.0001), SLC26A9 (P<0.0001) and SLC9A3 (P=0.0001) (gene-based permutation P values in the French cohort are <0.001, 0.235 and 0.017, respectively; FIG. 41).

Next, testing the apical hypothesis as a whole (which excludes SLC6A14), GWAS-HD provided genome-wide significant evidence for association between MI and multiple constituents of the apical plasma membrane, permutation P=0.0002, testing all 3,814 SNPs from 155 apical genes jointly and not subject to multiple hypothesis testing (FIGS. 40a and 40b). Even with the exclusion of SLC26A9 (and SLC6A14) to minimize any selection-bias concern, the apical hypothesis remained significant (P=0.0058). Thus, GWAS-HD established the involvement of other genes coding for apical constituents that we were not sufficiently powered to identify based on individual SNPs.

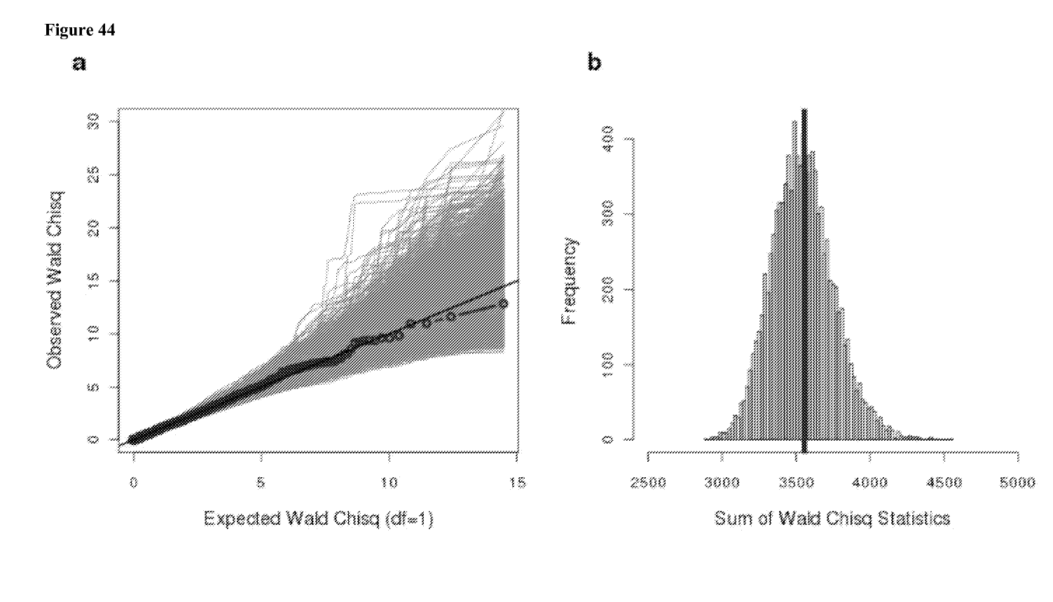

For comparison, a null hypothesis list of membrane-localized genes was constructed to test the GWAS-HD, for which we anticipated to see no relationship with MI. This list, also defined by GO annotation, comprised of all gene products present in the nuclear envelope (See Methods). The 224 nuclear envelope genes tagged by 3,537 GWAS SNPs showed no relationship with MI (permutation P=0.4639; FIG. 44).

The French cohort with genome-wide data provided independent validation of the apical hypothesis through genome-wide significant replication (permutation P=0.022, testing all apical SNPs simultaneously, FIGS. 40b and 40c). There were a large number of genes with suggestive association evidence and their substantial consistency between the North American and French samples (FIG. 41).

To determine the degree of involvement from the apical constituents and GWAS findings, Lasso was used to jointly analyze all 3,740 SNPs tagging the apical membrane genes (which include SLC26A9 and SLC9A3), and SLC6A14 (See Methods). Forty-eight SNPs spanning 36 different genes were retained by Lasso in the multivariate regression model (FIG. 41). These SNPs jointly explain an appreciable amount (˜17%, ˜2.5-fold increase from the GWAS alone) of ‘missing heritability’ in MI, and indicated that the GWAS-HD framework yielded considerable additional information leading to interpretations beyond single SNP or single gene associations.

### Methods

Human Subjects.

Consent was obtained from all participants of the North American Cystic Fibrosis Gene Modifier Consortium (NACFGMC) with procedural approval from the Institutional Review Boards of Johns Hopkins University (JHU), the University of North Carolina at Chapel Hill (UNC) and Case Western Reserve University (CWRU) and the Research Ethics Board of The Hospital for Sick Children. Consent was also obtained for participants from France with procedural approval (CPP n° 2004/15) and information collection approval by CNIL (n° 04.404).

Recruitment and Inclusions.

Cystic fibrosis (CF) patients and CF-related phenotypes including lung function⁵ and meconium ileus (MI) were collected by the NACFGMC. The Genetic Modifier Study (GMS) included two sets of samples, one ascertained on the phenotype of extremes of lung disease (GMS-lung) and the other on the presence of CF-related severe liver disease (GMS-liver). The MI GWAS was restricted to subjects with ‘severe’ (pancreatic exocrine insufficient) CFTR genotypes and of Caucasian background (see quality control below). Participants (the 1,140 CF patients not used in the initial GWAS) for the North American replication corresponded to the continuing collections at all sites (351 from JHU Twin and Sibling Study (TSS), 448 from UNC/CWRU and 341 from CGS) with known MI status based on previously described criteria and as rigorously defined in source documentation and/or evidence of an abdominal scar.

There are 49 CF centers in France, caring for an estimated 5,000 to 6,000 CF patients. In 2006, prospective enrollment of CF patients was initiated in 38 of 49 CF centers. In January 2011, phenotypic information was available for 2,898 patients. Those selected for genotyping comprising the French replication cohort included 1,362 CF patients who were enrolled before June 2010, all of whom are over 6 years of age with two severe CFTR mutations and both parents born in a European country.

Genotyping.

NACFGMC GWAS subjects were genotyped simultaneously using the Infinium HD Illumina 610-Quad BeadChip platform at McGill University and the Genome Quebec Innovation Center.

North American Replication Cohort.

DNA was extracted from whole blood or transformed lymphocytes quantified with fluorimetry. Genotyping was performed with allele-specific fluorescent probes in Taqman® SNP Genotyping Assays (Custom or On-Demand; Applied Biosystems) as recommended using a 96-well format.

French Replication Cohort.

DNA was obtained from whole blood and hybridized to the Illumina CNV370-Duo BeadChip for the first 299 patients (included before June 2009) and the Illumina 660W-Quad BeadChip for the remaining patients at the Centre National de Genotypage (CNG), Evry, France.

Quality Control. GWAS Genotypes.

Samples with genotype missing rate >10%, heterozygosity proportion <28%, sex incongruity, and patients of non-Caucasian ancestry as determined by the principle component (PC) analysis using EIGENSTRAT were excluded. Using IBD estimates from PLINK and PREST-plus, twelve cryptic full-sib pairs were identified and adjusted for relationships. Further, only one randomly selected individual from each of the 10 cryptic MZ pairs was retained, and parents of two cryptic parent-offspring pairs were deleted. In total, 3,763 samples were used for the analysis. SNPs with genotype call missing data rate >10%, MAF<2% were excluded, and 543,927 SNPs remained in the analysis.

North American Replication Genotypes.

End-point fluorescence was measured with the plate reader component of the 7900HT Real Time PCR System (Applied Biosystems) and aided by Taqman® Genotyper software for allele discrimination with call rates >95%. Two percent of samples were run in duplicate and 1% of the samples corresponded to individuals used in the initial GWAS to assure quality control and permit assessment across genotyping platforms, respectively, with concordances >99%.

French Replication Genotypes.

Patients with genotyping success rate <95%, sex incongruity, and pair-wise IBD estimates >40% were excluded, yielding a final set of 1,300 patients among which 1,232 had phenotype information for MI. SNPs present only on the CNV370-Duo chip were excluded from the analysis. SNPs with chip-wise missing data rate >10%, MAF<6% were excluded from the analysis. Overall, 554,792 SNPs were kept for the analysis, of which 256,756 were typed on both chips and 298,036 only on the 660W-Quad chip.

GO Annotation of the Apical Membrane Constituent and Nuclear Envelope Lists.

The

AmiGO tool¹³ (version 1.7) based on the Gene Ontology data was used to generate the two lists. A list of 157 apical genes was generated (retrieved Mar. 28, 2010; GO:00163245) by the cell location search phrase “apical plasma membrane” with restriction to Homo sapiens (SLC6A14 not on the list). In total 3,814 GWAS SNPs are within ±10 kb of the boundaries of 155 genes (NCBI36/hgl8); two genes are not tagged by any of the genotyped SNPs after QC. A list of 231 nuclear genes was generated (retrieved Apr. 17, 2010; GO:0005635) by the cell location search phrase “nuclear envelope” with restriction to Homo sapiens. This list consisted of all gene products associated with the nuclear membrane. In total 3,537 GWAS SNPs are within ±10 kb of the boundaries of 224 genes.

Imputation.

Using MACH, genotype imputation was conducted in two regions (SLC6A14 on chromosome X and SLC26A9 on chromosome 1) for the 3,763 subjects. The reference sample was the 90 CEU subjects extracted from the EUR continental group of the 1000 genomes August 2010 release provided in the four-site (Broad Institute, Michigan University, Boston College and NCBI) merged dataset. Imputation yielded genotype data for 250 chromosome X SNPs and 2,639 chromosome 1 SNPs, among which 175 and 183 SNPs with estimated imputation accuracy >0.3 (using MACH's R-squared accuracy measure) were considered for the association analysis. In SLC26A9, the best imputed SNP was only marginally more significant than any genotyped (6.23×10⁻⁹ vs. 9.88×10⁻⁹), while in SLC6A14 the minimum P value was provided by rs3788766, one of the genotyped SNPs.

Statistical Methods. Association Analysis.

Generalized estimating equations (GEE⁶) was used for GWAS with an exchangeable correlation structure to account for the full-sib relationship in the data (Geeglm function in R, version 2.9.2). Genotypes were coded additively for autosomal SNPs and chromosome X SNPs in females. In males, 0 and 2 were used for chromosome X SNPs. A site covariate with four levels (CGS, GMS-lung, GMS-liver, and TSS) was included. Logistic regression in a sample of 3,199 unrelated individuals with a site covariate and the first seven principal components was also conducted, and results are consistent with the analysis of the full 3,763 subjects. (Therefore the PCs were not included in the subsequent analysis and permutation tests.) The French GWAS used logistic regression with additive genotype coding (PLINK v1.07 for autosomal SNPs and R for X chromosome SNPs).

GWAS-HD, SFDR and Permutation.

GWAS-HD was used to accomplish two tasks: (1) to establish significance of individual SNPs at the genome-wide level after weighting according to a particular hypothesis, and (2) to test the significance of the hypothesis itself, by assessing whether the group of SNPs defined by the hypothesis display significantly smaller P values than would be expected under the null of no association.

To carry out the first task, GWAS SNPs were assigned to a high priority group (SNPs from the genes on the apical gene list) or a low priority group (all other SNPs). Stratified FDR control (SFDR) was then applied and q values were calculated separately in each group. Statistical significance at a given SNP was concluded if its q value was less than 0.05; each SNP was re-ranked genome-wide according to its new q value (the original GWAS P values were used to guide order if q values of two SNPs were identical).

To carry out the second task, that is to determine the statistical significance of the apical hypothesis involving 3,814 SNPs simultaneously (or 3,420 SNPs in the French replication cohort), the MI phenotype was permuted (to preserve the LD pattern between SNPs) within each consortium site and independently 10,000 times (or 1,000 in the French cohort). For each permutation sample, corresponding association analysis was performed, and a sum of the Wald association statistics of the 3,814 (or 3,420) SNPs was obtained. The empirical P value for the significance of the apical hypothesis was calculated as the number of permuted samples whose sum statistics were larger than that in the observed data, divided by 10,000 (or 1,000).

Gene-Based Analysis.

The analysis is similar to the permutation test above, but the sum statistic was obtained across all SNPs within ±10 kb of the boundaries of each gene. In total, 156 gene-based permutation tests were performed (155 apical genes and SLC6A14), and a conservative Bonferroni adjusted statistical significance level is Error!

Objects Cannot be Created from Editing Field Codes.

Lasso.

To determine which SNPS/genes jointly contribute to MI susceptibility, a multivariate analysis using penalized logistic regression (Lasso) was performed on 3,199 unrelated individuals (574 MI cases) extracted from the original 3,763 MI GWAS sample. The 3,814 SNPs from the apical plasma membrane list together with 15 SNPs within SLC6A14 were considered in the joint analysis (all SNPs with MAF>2%). After removing 93 SNPs in perfect LD with one another (SNPs with r²=1), a total of 3,740 SNPs were included as predictors in the Lasso analysis. The multivariate model also included the site covariate. The glmnet package in R was used to implement the Lasso. The default option in glmnet to standardize all predictors was turned off. The optimal value of the tuning parameter λ was chosen based on 10-fold cross-validation (CV) to maximize the deviance. Because the CV procedure randomly partitions the original data into training and testing sets, the optimal value of λ varies depending on how the data is split; we therefore repeated the 10-fold CV 50 times, and determined the optimal value of λ by examining the distribution of 50λ values and choosing the mode.

Estimating the Phenotypic Variance.

Pseudo R-squared was used as an estimate of the phenotypic variance explained by the SNPs of interest. Calculations used the lrm function in R by regressing MI on SNPs in FIG. 36 (˜5%) and all SNPs selected by Lasso (˜17%).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org, the National Center for Biotechnology Information (NCBI) on the world wide web at ncbi.nlm.nih.gov, or miRBase on the world wide web at microrna.sanger.ac.uk.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of identifying a subject as having increased risk of severe lung disease comprising the step of detecting in a biological sample from the subject an allele of a single nucleotide polymorphism, wherein the allele of the single nucleotide polymorphism is selected from the group consisting of:
a) a C allele at single nucleotide polymorphism rs 12793173;
b) an A allele at single nucleotide polymorphism rs1403543;
c) a C allele at single nucleotide polymorphism rs9268905;
d) a G allele at single nucleotide polymorphism rs4760506;
e) a T allele at single nucleotide polymorphism rs12883884;
f) an A allele at single nucleotide polymorphism rs12188164;
g) a C allele at single nucleotide polymorphism rs11645366; and
h) any combination thereof,
thereby identifying the subject as having increased risk of severe lung disease.

2. The method of claim 1, further comprising the step of determining whether the biological sample lacks a wild-type CFTR gene.

3. The method of claim 1, wherein the subject lacks a wild-type CFTR gene.

4. The method of claim 1, wherein the subject has or is suspected of having cystic fibrosis.

5-10. (canceled)

11. A method of identifying subject as a carrier of an allele of a gene associated with severe lung disease comprising the step of detecting in a biological sample from the subject a variant in a gene selected from the group consisting of: EHF, APIP, MC3R, CASS4, AURKA, CBLN4, C20orf106 and CSTF1, thereby identifying the subject as a carrier of an allele of a single nucleotide polymorphism associated with severe lung disease.

12. The method of claim 11, further comprising the step of determining whether the biological sample comprises a CFTR gene mutation.

13. The method of claim 11, wherein the subject is or is suspected of being a carrier of a mutated CFTR gene.

14. The method of claim 11, wherein the subject has at least one family member that has or is suspected of having cystic fibrosis.

15. The method of claim 11, wherein the step of detecting comprises performing a hybridization assay.

16. The method of claim 11, wherein the step of detecting comprises performing a nucleic acid amplification assay.

17. The method of claim 11, wherein the step of detecting comprises performing a nucleic acid sequencing assay.

18. The method of claim 11, further comprising the step of obtaining the biological sample from the subject.

19-30. (canceled)

31. A method of identifying a subject as having increased risk of having cystic fibrosis liver disease (CFLD) comprising the step of detecting in a biological sample from the subject an allele of a single nucleotide polymorphism, wherein the allele of the single nucleotide polymorphism is selected from the group consisting of:
a) a C allele at single nucleotide polymorphism rs914232;
b) a T allele at single nucleotide polymorphism rs2330183;
c) a T allele at single nucleotide polymorphism rs2838956;
d) a G allele at single nucleotide polymorphism rs1051266;
e) a T allele at single nucleotide polymorphism rs4819130;
f) a G allele at single nucleotide polymorphism rs3788190;
g) a T allele at single nucleotide polymorphism rs2236483;
h) a C allele at single nucleotide polymorphism rs2838950;
i) a G allele at single nucleotide polymorphism rs12483377;
j) a C allele at single nucleotide polymorphism rs3753019; and
k) any combination thereof,
thereby identifying the subject as having increased risk of having CFLD.

32. The method of claim 31, further comprising the step of determining whether the biological sample lacks a wild-type CFTR gene.

33. The method of claim 31, wherein the subject lacks a wild-type CFTR gene.

34. The method of claim 31, wherein the subject has or is suspected of having cystic fibrosis.

35-60. (canceled)

61. A method of identifying a subject as having increased risk of meconium ileus (MI) comprising the step of detecting in a biological sample from the subject an allele of a single nucleotide polymorphism, wherein the allele of the single nucleotide polymorphism is selected from the group consisting of:
a) a C allele at single nucleotide polymorphism rs7512462;
b) a G allele at single nucleotide polymorphism rs7415921;
c) a G allele at single nucleotide polymorphism rs4077468;
d) a T allele at single nucleotide polymorphism rs4077469;
e) a G allele at single nucleotide polymorphism rs12047830;
f) an A allele at single nucleotide polymorphism rs7419153;
g) a T allele at single nucleotide polymorphism rs10179921;
h) a T allele at single nucleotide polymorphism rs4684689;
i) an A allele at single nucleotide polymorphism rs17563161;
j) a T allele at single nucleotide polymorphism rs3788766;
k) a C allele at single nucleotide polymorphism rs5905283;
l) a G allele at single nucleotide polymorphism rs12839137; and
k) any combination thereof,
thereby identifying the subject as having increased risk of having MI.

62. The method of claim 61, further comprising the step of determining whether the biological sample lacks a wild-type CFTR gene.

63. The method of claim 61, wherein the subject lacks a wild-type CFTR gene.

64. The method of claim 61, wherein the subject has or is suspected of having cystic fibrosis.

65-90. (canceled)
